(19) United States
(12) Patent Application Publication
(10) Pub. No.: US 2026/0256738 A1
(43) Pub. Date: Sep. 3, 2026

(54) METHODS OF TREATING CANCER WITH A RAS MUTATION

(71) Applicant: Immuneering Corporation, Cambridge, MA (US)

(72) Inventors: Brett Matthew Hall, San Diego, CA (US); Peter John King, San Diego, CA (US); Praveen Nair, San Diego, CA (US); Jason Funt, San Diego, CA (US); Sarah Kolitz, Cambridge, MA (US)

(21) Appl. No.: 19/161,604

(22) PCT Filed: Mar. 1, 2024
(86) PCT No.: PCT/US2024/018246

§ 371 Date: Sep. 2, 2025

Related U.S. Application Data
(60) Provisional application No. 63/488,459, filed on Mar. 3, 2023.
(60) Provisional application No. 63/496,630, filed on Apr. 17, 2023.
(60) Provisional application No. 63/506,011, filed on Jun. 2, 2023.
(60) Provisional application No. 63/509,944, filed on Jun. 23, 2023.

(51) Int. Cl. A61K 31/37; A61P 35/00

(52) U.S. Cl. CPC A61K 31/37; A61P 35/00

(57) ABSTRACT

Methods of treating RAS-mutated cancers are provided, comprising the administration of a therapeutically effective amount of dual-MEK inhibitor with a short plasma half-life that inhibits both the phosphorylation of mitogen-activated protein kinase kinase 1 or 2 (pMEK) and the phosphorylation of mitogen-activated protein kinase 1 or 2 (pERK), wherein the dual-MEK inhibitor is administered to the patient in certain dose amounts that achieves a Cmax effective to inhibit pERK and pMEK and with an interval between consecutive doses that is at least about certain times the plasma half-life of the dual-MEK inhibitor. In some embodiments, the dual-MEK inhibitor is Compound A

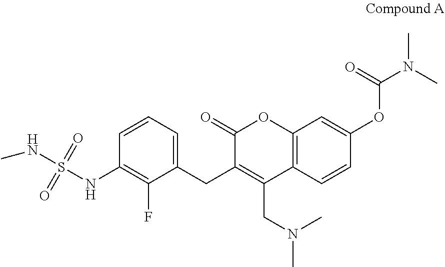

or a pharmaceutically acceptable salt thereof.

### INCORPORATION BY REFERENCE TO ANY PRIORITY APPLICATIONS

This patent application is a U.S. National Phase of International Application No. PCT/US2024/018246, filed Mar. 1, 2024 and claims the benefit of, and priority to, U.S. Provisional Patent Application No. 63/488,459, filed Mar. 3, 2023; U.S. Provisional Patent Application No. 63/496,630, filed Apr. 17, 2023; U.S. Provisional Patent Application No. 63/506,011, filed Jun. 2, 2023; and U.S. Provisional Patent Application No. 63/509,944, filed Jun. 23, 2023, each of which are incorporated by reference herein in their entirety.

### TECHNICAL FIELD

The present disclosure relates to the fields of chemistry and medicine. More particularly, the present disclosure relates to methods of treatment of certain forms of cancer with MEK inhibitor therapies, such as methods of treating cancer with a RAS mutation.

### BACKGROUND

In all cells, signaling pathways govern how cells regulate themselves as well as direct activities in relation to other cells in the body. One of the most commonly altered signaling pathways in cancer is the Mitogen Activated Protein Kinase (MAPK) pathway. RAS is a family of related oncogenes found upstream in the MAPK pathway that codes for four highly related protein isoforms, HRAS, NRAS, KRAS4A and KRAS4B. There are three RAS genes: kirsten-RAS (KRAS), neuroblastoma-RAS (NRAS), and harvey-RAS (HRAS), encoding four RAS proteins (KRAS4A, KRAS4B, NRAS, and HRAS). Among RAS genes, KRAS could encode two proteins (KRAS4A and KRAS4B) due to alternative RNA splicing.

In many solid tumors, the MAPK pathway is inappropriately activated, often through mutations in the key members of the pathway, including RAS. When RAS is switched “on” through the activation of the membrane-bound receptor tyrosine kinase, or RTK, the MAPK pathway functions to drive cell proliferation, differentiation, survival, and a variety of other cellular functions that are critical for the formation of tumors. Oncogenic RAS family mutations have been found in about one quarter of all human cancers, however cancers characterized by a RAS mutation are challenging to treat using known therapies. In addition, RAS mutation is a negative predictor of cancer patient survival; cancer patients have much poorer prognostic outcomes (or shorter overall survival) when their cancer has a RAS mutation. Thus, there is a dire unmet need for a more effective treatment for cancer associated with oncogenic RAS gene mutation.

Through widespread adaptation of molecular profiling, it is now recognized that up to one in two cancer patients harbor tumors which are inappropriately activated through the MAPK pathway. Many of these patients display tumors with activation mutations in RAS or RAF, which lie upstream of MEK and ERK. Because inappropriate activation of the MAPK pathway supports many of the core tumor capabilities described above, efforts to create new therapeutics to target this pathway have been a high priority in cancer drug research. However, to date nearly all therapeutics that target the MAPK pathway have been limited to narrow patient subpopulations and have struggled to balance tolerability, durability of response, and efficacy. Nearly all targeted therapeutics against the MAPK pathway have been designed for sustained pathway suppression, which has resulted in on-target drug-related toxicities that limit clinical durability and potential drug-drug combinations.

Activating mutations of RAS and/or RAF in the MAPK pathway are observed in approximately 30% of all cancer patients, and inappropriate activation of this pathway is observed in up to 50% of all tumors and represents one of the most highly utilized signaling pathways in oncology. In aggressive solid tumors of the pancreas, skin, lungs and colon, mutations in RAS and/or RAF are even more common. For example, approximately 40% of lung cancers and approximately 95% of pancreatic cancers are due to RAS and/or RAF mutations, and approximately 50% of melanomas, 40% of thyroid and 30% of ovarian tumors display activation mutations, mainly, in BRAF but also in ARAF and CRAF (RAF1). To date, mitogen-activated extracellular signal-regulated kinase (MEK) inhibitors approved by the Food and Drug Administration (FDA) have been ineffective at treating RAS mutant tumors likely attributable to well-known mechanisms of resistance, including C-isomer of rapidly accelerated fibrosarcoma (CRAF)-mediated MEK (re)activation, or CRAF-bypass.

Traditional targeted oncology drug development generally seeks to chronically sustain pathway inhibition, by prioritizing drugs with long to moderate half-lives and dosing them at intervals that maintain a sufficient level of drug, even at trough, to maintain target occupancy. This prevailing chronic ablation approach can cause on-target drug-related toxicity and limit clinical durability as a result of drug holidays, treatment discontinuation, and/or adaptive resistance. To date, FDA-approved MEK inhibitors have been ineffective at treating RAS mutant tumors when compared to BRAF mutant tumors because of a well-known mechanism of resistance, CRAF-mediated MEK reactivation, also known as CRAF-bypass. In addition, a well-known limitation of current FDA approved MEK inhibitors are high rates of serious drug-related adverse events, or SAE's, most often observed in over 50% of treated patients, which leads to drug intolerability. The longer half-life of these drugs (e.g., up to 2 to 4 days) with once per day or less frequent dosing, or moderate half-life (e.g., 3 to 6 hours) with increased dosing frequency, contributes to high rates of adverse events because these drugs systemically circulate for an extended period of time harming healthy normal cells, which also rely on the pathway for survival.

Developing novel therapeutics to effectively, broadly and safely target this pathway may provide clinical benefit in large patient populations with significant unmet needs. In addition, although this pathway represents one of the most active areas in cancer drug discovery and development, targeted therapeutics that more effectively and safely normalize, but not chronically ablate, ERK signaling may uncouple drug activity and tolerability, while optimizing both. There remains an unmet medical need for methods of treating certain forms of cancer activated through the MAPK pathway, including methods of treatment to cyclically disrupt the ERK and MEK with a MEK inhibiting compound having a shorter half-life than prior “first generation” MEK inhibitor compounds such as trametinib, cobimetinib, binimetinib, and selumetinib. In addition, there is a clear unmet medical need and limited treatment options available to patients with RAS-mutated solid tumors.

Previous MEK inhibitors have been approved for BRAF-mutated lung cancer and melanoma; however, to reduce cytoreduction in the malignant clones, they must sustain the effective target occupancy across the dosing interval, and this chronic pathway ablation can be associated with on-target toxicities in normal cells. First-generation MEK inhibitors were designed to prevent phosphorylation of ERK (i.e., a surrogate for activation) by MEK through therapeutic engagement of an allosteric pocket adjacent to the ATP binding site, but first-generation compounds fail to control MEK phosphorylation at the RAF activation domain (e.g., Serine 218 and Serine 222 on MEK1), which is the initial step of MEK activation. Following treatment of RAS mutant cells, an increased accumulation of cytoplasmic pMEK contributes, in part, to the dogma that MEK inhibitors must sustain chronic target occupancy in order to inhibit the MAPK pathway effectively. However, chronic MEK inhibition leads to narrow therapeutic windows, significant class-effect toxicities and acquired or adaptive resistance of tumor cells. All of the first generation MEK inhibitors are designed for prolonged systemic circulation, either through longer half-life (e.g., up to 2 to 4 days), or moderate half-life (e.g., 3 to 6 hours) combined with increased dosing frequency. In either case, the prolonged systemic circulation contributes to high rates of adverse events by harming healthy normal cells, which also rely on the MAPK pathway. While significant advances have been made to better understand the underlying biology of the MAPK pathway, drugs designed to counteract these perturbations in the signaling pathway continue to be hindered by excess drug-related toxicity, short-lived durability of clinical response, and limited numbers of biomarker positive patients for mutation-specific drugs.

There remains an unmet medical need for methods of treating certain forms of cancer activated through the MAPK pathway, including methods of treatment to cyclically disrupt the ERK and MEK with a MEK inhibiting compound having a shorter half-life than prior “first generation” MEK inhibitor compounds such as trametinib, cobimetinib, binimetinib, and selumetinib.

### SUMMARY OF THE DISCLOSURE

In some embodiments, methods of therapeutically treating a cancer characterized by expressing a mutant form of a RAS protein comprises administering a dual-MEK inhibitor to a subject in need thereof. In some embodiments, methods of treatment comprise the administration of a dual-MEK inhibitor to achieve deep cyclic inhibition of the MAPK pathway. Deep cyclic inhibition is a novel therapeutic approach for treatment of certain forms of cancer that aims to deprive tumor cells of the sustained proliferative signaling required for rapid growth, while sparing healthy cells through a cadenced, normalized level of signaling. In some embodiments, deep cyclic inhibition can be achieved by administering a dual-MEK inhibitor having sufficiently high pharmacodynamic inhibition of pERK (e.g., 90%+) and sufficiently short plasma half-life (e.g., 2 hours) at sequential dose intervals that are about seven or more times the plasma half-life (e.g., once daily).

In some embodiments, methods of treatment provide methods of administering a dual-MEK inhibitor having a high Cmax and a short plasma drug half-life at sequential doses that are administered at a dose interval (i.e., time between doses) that is greater than the plasma drug half-life. For example, in some embodiments, a method of treatment comprises administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are at least about 7-10 times the duration of the plasma drug half-life.

In some embodiments, methods provided herein comprise the use of and/or a method of treatment comprising administering Compound A or a pharmaceutically acceptable salt thereof to a subject in need thereof,

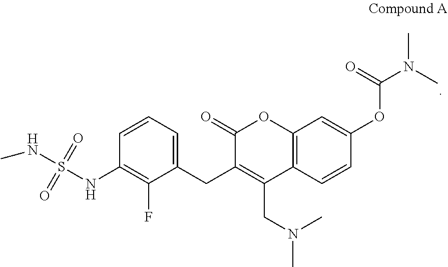

Compound A can be administered to rapidly reach a high Cmax and has a sufficiently short half-life to achieve a deep cyclic inhibition pathway for the treatment of patients with RAS-mutated solid tumors. Compound A has an extremely short half-life, which allows for chronic dosing while maintaining a near-zero drug trough between doses on a 24-hour cycle, and this unique pharmacokinetic profile is expected to improve tolerability by inhibiting MAPK signaling cyclically rather than ablating it chronically. Compound A is characterized by a sufficiently short plasma half-life to reduce sustained pathway inhibition.

Unlike previous MEK inhibitors, Compound A rapidly reaches maximal drug exposure while achieving negligible drug trough levels within 12- to 24-hour cycles. This allows Compound A to be dosed chronically on a daily schedule to drive deep, cyclic inhibition of the MAPK pathway. By also engaging the RAF activation loop on MEK, Compound A prevents RAF-mediated MEK activation and further disrupts KSR. By designing Compound A to rapidly reach a high Cmax and have a short half-life to inhibit the activation of ERK while preventing RAF-mediated pathway reactivation, Compound A presents a novel approach to targeting MEK that may allow for broader activity and improved tolerability in patients with RAS-mutated solid tumors when compared with currently approved MEK inhibitors. As described herein, preclinical studies demonstrate well-tolerated anti-tumor activity profiles of Compound A in both the RAS- and RAF-mutant tumor setting.

In some embodiments, a dual MEK1/2 inhibitor may be useful to inhibit the activation of ERK while preventing RAF-mediated pathway reactivation. In some embodiments, the dual MEK1/2 inhibitor is Compound A. In some embodiments, Compound A is useful for the treatment of certain forms of cancer. In some embodiments, a method of treating a patient with a RAS-mutated or RAF-mutated cancer, comprises administering a therapeutically effective amount of Compound A to a patient in need thereof. In some embodiments, a method of treating a patient with a RAS-mutated or RAF-mutated cancer, comprises orally (p.o.) administering a therapeutically effective amount of Compound A to a patient in need thereof. In some embodiments, a method of treating a patient with a RAS-mutated or RAF-mutated cancer, comprises orally (p.o.) administering a therapeutically effective amount of Compound A once per day (QD) to a patient in need thereof. In some embodiments, Compound A is an oral, dual MEK1/2 inhibitor useful as monotherapy and in combination with approved agents for the treatment of solid tumors.

In some embodiments, methods of treatment comprise orally administering Compound A once-daily to a patient in need thereof in a total daily dose of 160 mg to treat certain forms of cancer. In some embodiments, methods of treatment comprise orally administering Compound A once-daily to a patient in need thereof in a total daily dose of 240 mg to treat certain forms of cancer. In some embodiments, methods of treatment comprise orally administering Compound A once-daily to a patient in need thereof in a total daily dose of 320 mg to treat certain forms of cancer.

In some embodiments, a dual-MEK inhibitor can be administered to solid tumor patients, specifically those with inappropriate activation of the MAPK pathway. Compound A has been specifically designed to provide deep cyclic inhibition of the MAPK pathway with a once-daily oral dosing cadence. Compound A was designed to overcome MAPK-feedback loops and to have an intentionally short plasma half-life, which is believed could enable it to provide deep cyclic inhibition. Without wishing to be bound by theory, it is believed that Compound A has the potential to provide broader therapeutic activity and an improved tolerability profile relative to known MEK inhibitors and that Compound A has the potential to target patients with solid tumors driven by any mutation in KRAS, NRAS, or HRAS.

In some embodiments, Compound A is useful for the treatment of patients with certain forms of cancer, including pancreatic, melanoma, colorectal and non-small cell lung cancer, or NSCLC, caused by mutations of RAS. Methods of treatment comprising the administration of Compound A for the treatment of patients diagnosed with certain forms of cancer are provided. In some embodiments, Compound A is administered as a once-daily oral monotherapy for the treatment of RAS mutant tumors. In some embodiments, methods of administering Compound A can achieve a pharmacokinetic profile in a patient that is characterized by sufficiently high Cmax to provide stronger inhibition of the MAPK pathway than has been observed with first and second generation MEK inhibitors, followed by a near-zero drug trough.

The disclosure also provides the discovery that Compound A was observed to bind to mitogen-activated protein kinase kinase, or MEK, and acts as a highly selective inhibitor of mitogen-activated protein kinase, or ERK, activation (i.e., phosphorylation), with a “Dual MEK” function that is designed to block the CRAF-bypass feedback to prevent MAPK pathway reactivation. Compound A is also designed to prevent RAF-mediated activation of MEK, such as CRAF-bypass, by engagement of the RAF activation loop on MEK, and at elevated levels further disrupt the kinase suppressor of RAS 1 and 2, or KSR.

As described herein, Compound A was evaluated in a range of human and murine solid tumor models, including those with activating MAPK pathway mutations in KRAS, NRAS, HRAS, BRAF, NF1 and KSR. For example, Compound A was evaluated in the MIA PaCa-2 (KRAS G12C PDAC), SK-MEL-2 (NRAS Q61R melanoma), A549 (KRAS G12S NSCLC) and Colon-26 (KRAS G12D CRC) mouse tumor models. Compound A was compared head-to-head to current FDA-approved MEK and BRAF inhibitors in KRAS-, NRAS- and BRAF-mutated solid tumor rodent models representing lung, colon, pancreas and skin cancers. In each of these studies, Compound A demonstrated tumor stasis or regression while maintaining body weight.

In some embodiments, methods of treatment comprising administering a therapeutically effective amount of Compound A to treat broad populations of solid tumor patients, specifically those with inappropriate activation of the MAPK pathway such as patients with solid tumors driven by any mutation in KRAS, NRAS, or HRAS.

In some embodiments, a method of treating a patient with a RAS- or RAF-mutated cancer, comprises administering a therapeutically effective amount of a compound having the structure of Compound A:

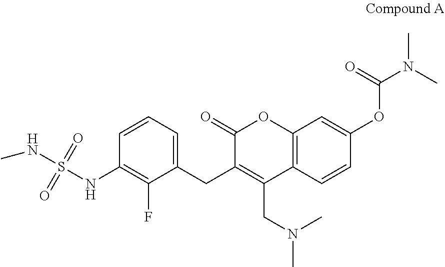

or a pharmaceutically acceptable salt thereof, to the patient in need thereof.

In some embodiments, Compound A is administered to the patient (a) in a dose amount that achieves a Cmax effective to inhibit pERK and pMEK and (b) with an interval between consecutive doses that is at least about seven times the plasma half-life of Compound A. In some embodiments, the method comprises administering Compound A to the patient as a monotherapy to treat the cancer. In some embodiments, a method of treating a patient with a RAS- or RAF-mutated cancer, comprising administering a therapeutically effective amount of a compound having the structure of Compound A or a pharmaceutically acceptable salt thereof, to the patient in need thereof, wherein the cancer expresses a KRAS mutation. In some embodiments, a method of treating a patient with a RAS- or RAF-mutated cancer, comprising administering a therapeutically effective amount of a compound having the structure of Compound A or a pharmaceutically acceptable salt thereof, to the patient in need thereof, wherein the cancer expresses a RAF mutation. In some embodiments, the Compound A is administered orally to the patient in need thereof.

In some embodiments, a method of treatment comprises administering to a patient total of 320 mg per day (QD) of Compound A to a patient in need thereof as a monotherapy for the treatment of a RAS- or RAF-mutated cancer. In some embodiments, a method of treatment comprises administering to a patient total of 240 mg or 320 mg per day (QD) of Compound A to a patient in need thereof in combination with another therapeutic agent for the treatment of a RAS- or RAF-mutated cancer.

### BRIEF DESCRIPTION OF THE DRAWINGS

FIGS. 1A and 1B are graphs summarizing results obtained from preclinical testing of Compound A in various xenograft tumor models (tumor volume), for pancreatic cancer (KRAS-G12C and KRAS-G12V), melanoma (NRAS-G61R), non-small cell lung cancer (NSCLC) (KRAS-G12S), and colorectal cancer (KRAS-G12D).

FIGS. 2A and 2B are graphs showing tumor pharmacodynamic transcriptome data from a head-to-head comparison of Compound A against selumetinib using a Colon-26 syngeneic tumor model.

FIG. 3 is a graph showing results from a head-to-head comparison of Compound A against four FDA-Approved MEK Inhibitors Using a A549 Tumor Cell Model, showing Compound A prevented downstream activation of ERK (↓ pERK) and inhibited activation of MEK (↓ pMEK).

FIG. 4A is a graph showing the tumor Pharmacokinetic and Pharmacodynamic (pERK) activity of Compound A in the Colon-26 animal model.

FIG. 4B is a graph showing the tumor pharmacodynamic transcriptome activity of Compound A in the Colon-26 animal model.

FIGS. 5A-C are graphs showing the head-to-head comparison of Compound A against binimetinib and selumetinib using a Colon-26 syngeneic tumor model (tumor volume).

FIGS. 6A and 6B are graphs showing data from head-to-head comparison of Compound A against binimetinib+/−encorafenib using an A549 xenograft tumor model (tumor volume).

FIGS. 7A and 7B are graphs showing data from the head-to-head comparison of Compound A against binimetinib+/−encorafenib using a A375 xenograft tumor model (tumor volume).

FIG. 8A are graphs showing data from the head-to-head comparison of Compound A+/−sotorasib (AMG-510) using a MIA PaCa-2 Xenograft Tumor Model (Tumor Volume).

FIG. 8B are graphs showing data from the head-to-head comparison of Compound A, sotorasib (AMG-510) and adagrasib using a CAPAN-2 Xenograft Tumor Model (Tumor Volume).

FIG. 9 are graphs showing results from a head-to-head comparison of tumor volume measured with Compound A against binimetinib in a NRAS-Q61R SK-MEL-2 melanoma xenograft study (Tumor Volume).

FIG. 10 is a graph showing data obtained from an evaluation of Compound A as compared to vehicle using a SK-MEL-2 xenograft tumor model (Tumor Volume).

FIG. 11A is a graph showing 3D-TGA Compound A dose responses calculated for sensitive tumor models.

FIG. 11B is a graph showing 3D-TGA Compound A dose responses calculated for intermediate tumor models.

FIG. 11C is a graph showing 3D-TGA Compound A dose responses calculated for resistant tumor models.

FIG. 12A is graph showing the mapping of biomarker sensitive patient populations (GENIE) for pancreatic cancer.

FIG. 12B is graph showing the mapping of biomarker sensitive patient populations (GENIE) for melanoma cancer.

FIG. 12C is graph showing the mapping of biomarker sensitive patient populations (GENIE) for lung cancer.

FIG. 12D is graph showing the mapping of biomarker sensitive patient populations (GENIE) for colorectal cancer.

FIG. 12E is graph showing the mapping of biomarker sensitive patient populations (GENIE) for prostate cancer.

FIG. 12F is graph showing the mapping of biomarker sensitive patient populations (GENIE) for breast cancer.

FIG. 12G is graph showing the mapping of biomarker sensitive patient populations (GENIE) for ovarian cancer.

FIG. 12H is graph showing the mapping of biomarker sensitive patient populations (GENIE) for glioblastoma cancer.

FIG. 13 is a table showing response and non response identified in analysis of 132 tumor models using humanized 3D-TGA.

FIG. 14A is a graph showing the pharmacokinetics (PK) and pharmacodynamics (PD) for pERK for Compound A in two human patients (3 and 4) diagnosed with colorectal cancer with a NRAS-Q61K mutation, after administration of 160 mg of Compound A administered orally (po) once daily (QD) (Dose Level III).

FIG. 14B is a graph showing the pharmacokinetics (PK) and pharmacodynamics (PD) for phosphorylated MEK (pMEK) for Compound A in two human patients (3 and 4) diagnosed with colorectal cancer with a NRAS-Q61K mutation, after administration of 160 mg of Compound A administered orally (po) once daily (QD) (Dose Level III).

FIG. 14C is a graph showing the modeling of PK and binding kinetics (BK) for Compound A in two human patients (3 and 4) diagnosed with colorectal cancer with a NRAS-Q61K mutation, after administration of 160 mg of Compound A administered orally (po) once daily (QD) (Dose Level III).

FIG. 14D is a graph showing PK/PD (pERK) for Compound A from patient 3 after receiving 160 mg Compound A administered orally (po) once daily (QD) (cycle 1, day 15).

FIG. 14E is a graph showing PK/PD (pMEK) for Compound A from patient 3 after receiving 160 mg Compound A administered orally (po) once daily (QD) (cycle 1, day 15).

FIG. 14F is a graph showing PK/PD (pERK) for Compound A from patient 4 after receiving 160 mg Compound A administered orally (po) once daily (QD) (cycle 1, day 15).

FIG. 14G is a graph showing PK/PD (pMEK) for Compound A from patient 4 after receiving 160 mg Compound A administered orally (po) once daily (QD) (cycle 1, day 15).

FIG. 15A is a graph showing PK/PD (pERK) for Compound A from patient 1 after receiving 40 mg Compound A administered orally (po) once daily (QD) (cycle 1, day 1).

FIG. 15B is a graph showing PK/PD (pMEK) for Compound A from patient 1 after receiving 40 mg Compound A administered orally (po) once daily (QD) (cycle 1, day 1).

FIG. 16A is a graph showing PK/PD (pERK) for Compound A from patients after receiving 80 mg Compound A administered orally (po) once daily (QD) (cycle 1, day 1).

FIG. 16B is a graph showing PK/PD (pMEK) for Compound A from patient 2 after receiving 80 mg Compound A administered orally (po) once daily (QD) (cycle 1, day 1).

FIG. 17A is a graph showing PK/PD (pERK) for Compound A from patient 5 after receiving 320 mg Compound A administered orally (po) once daily (QD) (cycle 1, day 1).

FIG. 17B is a graph showing PK/PD (pMEK) for Compound A from patient 5 after receiving 320 mg Compound A administered orally (po) once daily (QD) (cycle 1, day 1).

FIG. 17C is a graph showing PK/PD (pERK) for Compound A from patient 5 after receiving 320 mg Compound A administered orally (po) once daily (QD) (cycle 1, day 15).

FIG. 17D is a graph showing PK/PD (pMEK) for Compound A from patient 5 after receiving 320 mg Compound A administered orally (po) once daily (QD) (cycle 1, day 15).

FIG. 18A is a graph showing PK/PD (pERK) for Compound A from patient 6 after receiving 320 mg Compound A administered orally (po) once daily (QD) (cycle 1, day 1).

FIG. 18B is a graph showing PK/PD (pMEK) for Compound A from patient 6 after receiving 320 mg Compound A administered orally (po) once daily (QD) (cycle 1, day 1).

FIG. 18C is a graph showing PK/PD (pERK) for Compound A from patient 6 after receiving 320 mg Compound A administered orally (po) once daily (QD) (cycle 1, day 15).

FIG. 18D is a graph showing PK/PD (pMEK) for Compound A from patient 6 after receiving 320 mg Compound A administered orally (po) once daily (QD) (cycle 1, day 15).

FIG. 19A is a graph showing PK/PD (pERK) for Compound A from patient 7 after receiving 320 mg Compound A administered orally (po) once daily (QD) (cycle 1, day 1).

FIG. 19B is a graph showing PK/PD (pMEK) for Compound A from patient 7 after receiving 320 mg Compound A administered orally (po) once daily (QD) (cycle 1, day 1).

FIG. 19C is a graph showing PK/PD (pERK) for Compound A from patient 7 after receiving 320 mg Compound A administered orally (po) once daily (QD) (cycle 1, day 15).

FIG. 19D is a graph showing PK/PD (pMEK) for Compound A from patient 7 after receiving 320 mg Compound A administered orally (po) once daily (QD) (cycle 1, day 15).

FIG. 20A is a graph showing plasma concentration (PK) for Compound A from patient 13 after receiving 240 mg Compound A administered orally (po) once daily (QD) at cycle 1, day 1 and at cycle 1, day 15.

FIG. 20B is a graph showing PK/PD (pERK) for Compound A from patient 13 after receiving 240 mg Compound A administered orally (po) once daily (QD) at cycle 1, day 1 and at cycle 1, day 15.

FIG. 20C is a graph showing plasma concentration (PK) for Compound A from patient 16 after receiving 320 mg Compound A administered orally (po) once daily (QD) at cycle 1, day 1 and at cycle 1, day 15.

FIG. 20D is a graph showing PK/PD (pERK) for Compound A from patient 16 after receiving 320 mg Compound A administered orally (po) once daily (QD) at cycle 1, day 1 and at cycle 1, day 15.

FIG. 21 illustrates a distribution of RAS^{mut} across patients in the GENIE (v13.0) database.

FIGS. 22A-B illustrates mutation profiles across indications in GENIE (v13) patient database.

FIG. 23 illustrates representative subset of compound 9 dose responses (132 3D-TGAs). Cell lines tested in 3D-TGA were assigned response of sensitive (IC50<1 μM), intermediate (IC50≥1 and >25% reduction at 10 μM), and resistant otherwise (3D-TGA).

FIG. 24 illustrates a table of humanized 3D-TGA response: tumor tissue and RAS mutation status.

FIG. 25 illustrates a table of KRAS G12 variant and Associated 3D-TGA response category.

### DETAILED DESCRIPTION

Aspects of the disclosure provide for treating, modulating, preventing, reducing, or ameliorating a cancer in a subject. In some embodiments, the cancer is a solid tumor harboring a RAS mutation. In some embodiments, methods of treating, modulating, preventing, reducing, or ameliorating may include administering a dual-MEK inhibitor having a high Cmax and a short plasma drug half-life at sequential doses that are administered at a dose interval (i.e., time between doses) that is greater than the plasma drug half-life. For example, in some embodiments, a method of treating, modulating, preventing, reducing, or ameliorating may include administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are about 7-18 times the duration of the plasma drug half-life.

In some embodiments, the dual MEK inhibitor is Compound A and the method of treating, modulating, preventing, reducing, or ameliorating may include administering Compound A once-daily to a patient in need thereof, for the treatment of a solid tumor harboring RAS mutations.

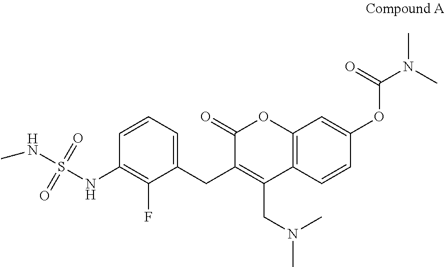

Compound A has a molecular weight of 506.55 g/mol and a molecular formula of C₂₃H₂₇FN₄O₆S. Compound A can be referred to as 4-((dimethylamino)methyl)-3-(2-fluoro-3-((N-methylsulfamoyl)amino)benzyl)-2-oxo-2H-chromen-7-yl dimethylcarbamate. The preparation of Compound A is described in Example 1.

Without wishing to be bound by theory, Compound A is a dual-MEK inhibitor that inhibits ERK and MEK. Compound A allosterically inhibits MEK by targeting the site lying adjacent to the binding pocket of adenosine triphosphate, or ATP, which would result in downstream inhibition of ERK.

In primary pharmacodynamic assays as described in Example 2, Compound A inhibited pERK across multiple tumor cells with half maximal inhibitory concentrations (IC₅₀) values ranging from 24.2 nM to 83.2 nM, with the most sensitive models being predominantly melanoma and pancreatic cancer cell lines that harbor activating MAPK pathway mutations in K isomer of rat sarcoma (KRAS), N isomer of rat sarcoma (NRAS), H isomer of rat sarcoma (HRAS), BRAF, neurofibromatosis type 1 (NF1), and/or kinase suppressor of Ras (KSR). Corresponding reductions in pMEK were observed with treatment of cells with 100 nM Compound A, confirming that Compound A was not sensitive to the MAPK pathway feedback reactivation events in RAS mutant models, such as CRAF-bypass.

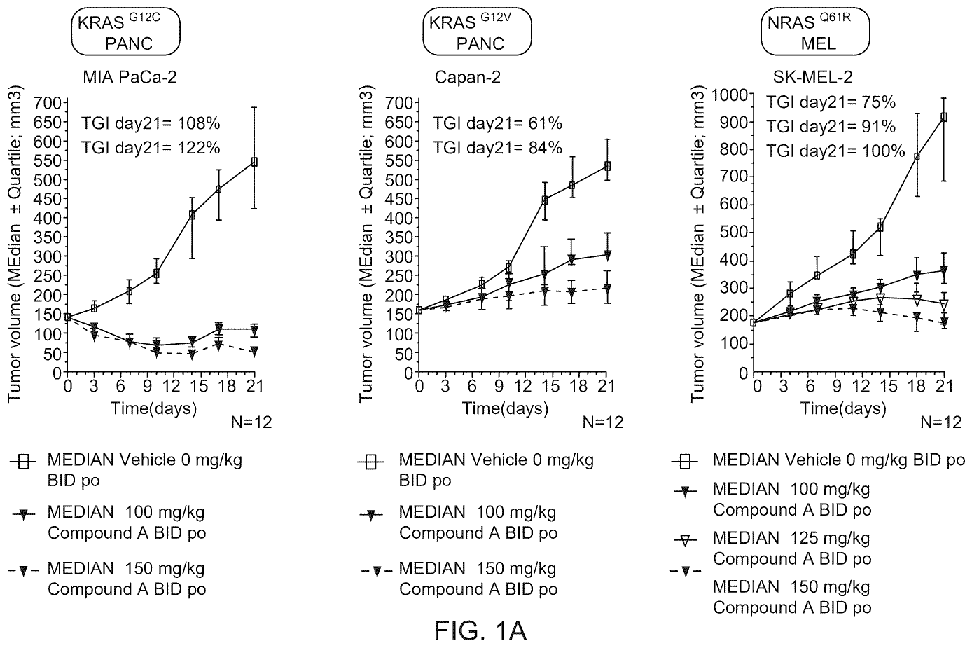

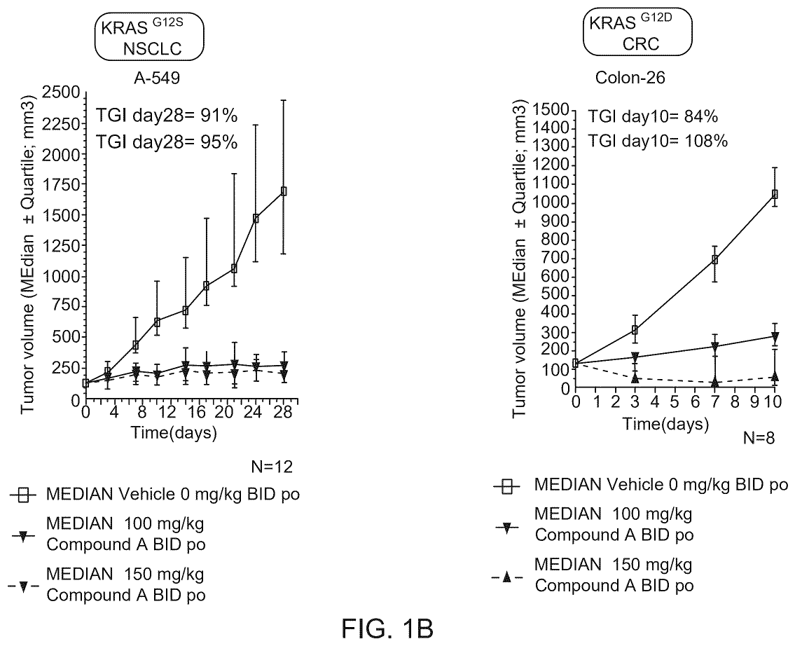

Compound A has been evaluated in a wide range of human and murine solid tumor models, including those with activating MAPK pathway mutations in KRAS, NRAS, HRAS, BRAF, NF1, and KSR. Among others, Compound A was investigated in MIA PaCa-2 (KRAS^{G12C} PDAC), Capan-2 (KRAS^{G12V} PDAC), SK-MEL-2 (NRAS^{Q61R} melanoma), A549 (KRAS^{G12S} NSCLC) and Colon-26 (KRAS^{G12D} CRC) mouse tumor models at various dose ranges (FIG. 1). Significant tumor reduction was observed at all doses and in all models, lending evidence that Compound A activity is not dependent upon specific RAS mutations or RAS paralogues.

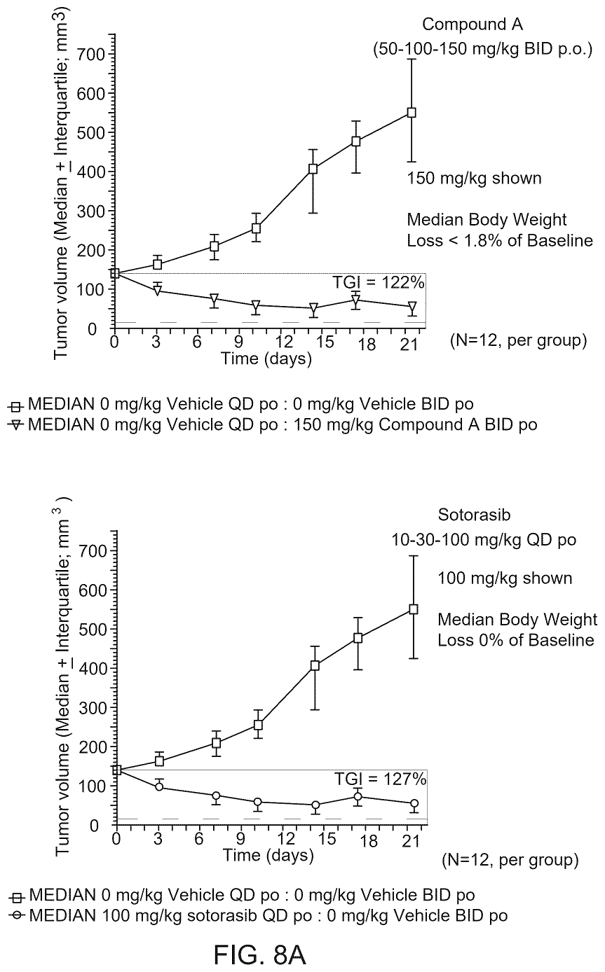

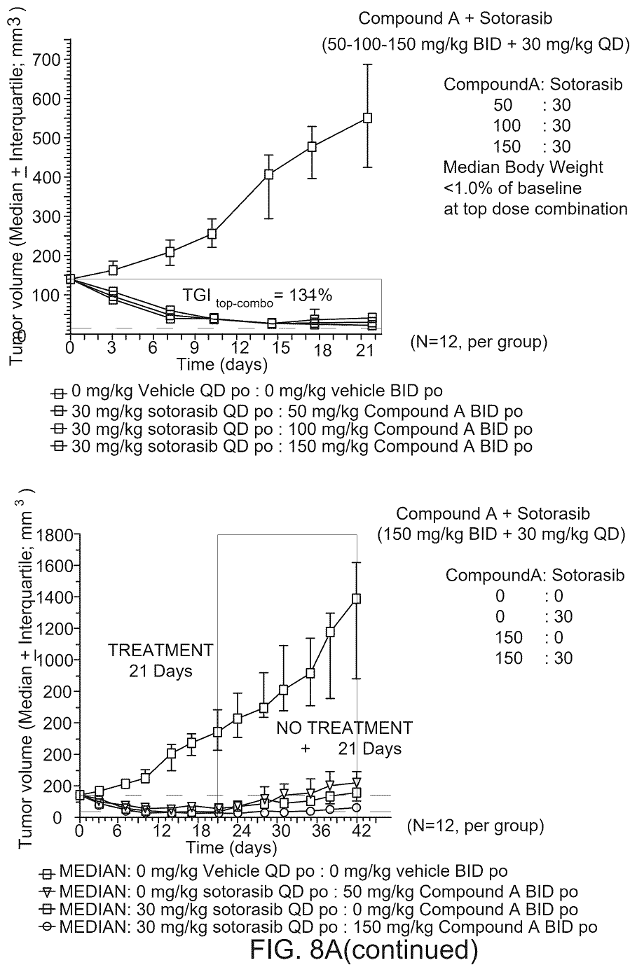

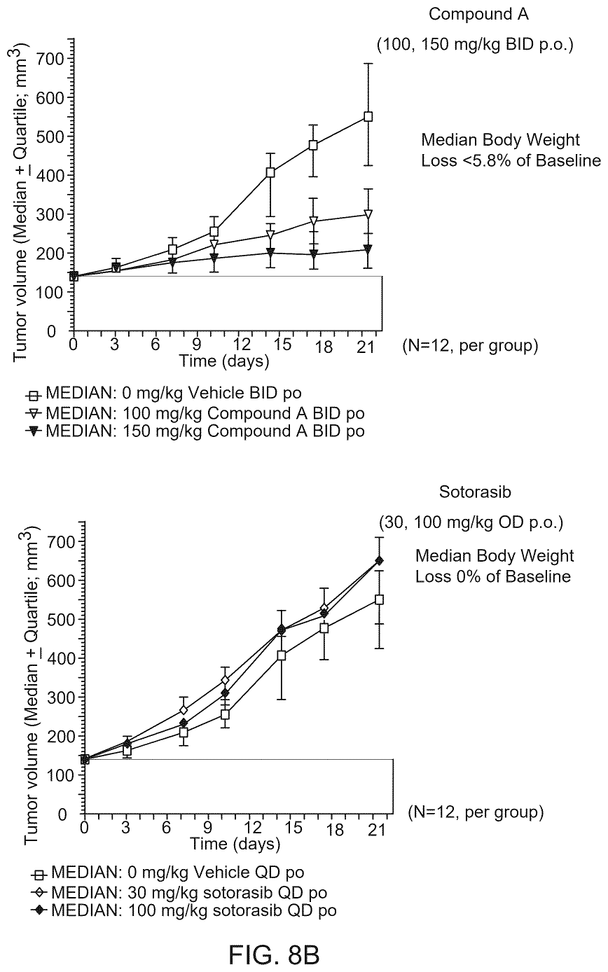

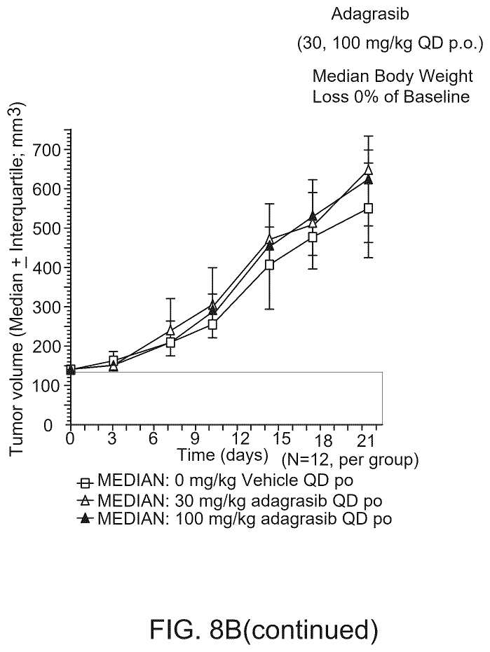

In head-to-head nonclinical studies, Compound A was compared to certain current FDA-approved MEK, BRAF and KRAS^{G12C} inhibitors in KRAS-, NRAS-, and BRAF-mutated solid tumor rodent models representing lung, colon, pancreas, and skin cancers; in each study, it demonstrated tumor stasis or regression while maintaining body weight. For instance, Compound A (100 and 150 mg/kg BID) was compared to the commercially available KRAS inhibitors sotorasib (30 and 100 mg/kg QD) and adagrasib (30 and 100 mg/kg QD) in the Capan-2 (KRAS^{G12D}) model (FIG. 8B), with Compound A showing efficacy in a non-KRAS^{G12C} model.

In the KRAS^{G12C} MIA PaCa-2 pancreatic model, similar tumor growth inhibition was observed between Compound A and sotorasib, and tumor regressions with improved durability of response (DoR) were demonstrated when Compound A and sotorasib were used in combination (FIG. 8A).

In some embodiments, methods of treatment provide methods of administering a dual-MEK inhibitor having a high Cmax and a short plasma drug half-life at sequential doses that are administered at a dose interval (i.e., time between doses) that is greater than the plasma drug half-life. For example, in some embodiments, a method of treatment comprises administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are about 7-18 times the duration of the plasma drug half-life. In some embodiments, Compound A has a short plasma half-life, with a near-zero steady state drug trough concentration to enable deep, cyclic inhibition of the MAPK pathway.

In some embodiments, methods of treatment comprise the administration of a dual-MEK inhibitor to achieve deep cyclic inhibition of the MAPK pathway. Deep cyclic inhibition is a novel therapeutic approach for treatment of certain forms of cancer that aims to deprive tumor cells of the sustained proliferative signaling required for rapid growth, while sparing healthy cells through a cadenced, normalized level of signaling. In some embodiments, deep cyclic inhibition can be achieved by administering a dual-MEK inhibitor having sufficiently high pharmacodynamic inhibition of pERK (e.g., ~90%) and sufficiently short plasma half-life (e.g., −2 hours) at sequential dose intervals that are about seven or more times the plasma half-life (e.g., once or twice daily).

In some embodiments, Compound A is a potent, non-ATP competitive, small molecule dual inhibitor of MEK. Not wishing to be bound by theory, Compound A may act as a dual MEKi with a new approach to MEK inhibition that is differentiated from first-generation MEKi by allosterically disrupting the activation of MEK itself, instead of inhibiting the ability of MEK to activate ERK. In contrast to first-generation MEK inhibitors, Compound A may block both MEK phosphorylation as well as downstream phosphorylation of ERK (i.e., dual MEK). It is believed that Compound A prevents RAF-mediated MEK activation and further disrupts KSR by also engaging the RAF activation loop on MEK. In contrast, first-generation MEK inhibitors are believed to engage the allosteric pocket of MEK to prevent the downstream phosphorylation of ERK. Phosphorylation of MEK through the RAF activation pathway is left unimpeded. Depleting intracellular pools of pERK is believed to remove the negative feedback signal on CRAF, which in turn is believed to hyperactivate phosphorylated MEK (pMEK) through CRAF leading to CRAF bypass.

Preclinical models suggest that deep, cyclic inhibition is achieved through an elevated plasma drug Cmax followed by a zero or near-zero drug trough within a 12- to 24-hour window, which requires a minimum of 7 to 8, but no more than 18, drug half-lives between doses. In addition to its novel mechanism of action that is resistant to CRAF bypass, Compound A has a short half-life (t_{1/2}) relative to other MEKi that allows for deep, cyclic inhibition of the MAPK pathway instead of chronic ablation. Chronic suppression of MAPK signaling is known to drive on-target toxicity for this drug class, but deep, cyclic inhibition with Compound A could improve tolerability. The concentration of Compound A over time in plasma and tumor follows parallel courses of rapid uptake after administration then a rapid decline to baseline, indicating plasma concentrations are a good reflection of concentrations in the tumor itself. Compound A has a very short half-life (1.2 to 1.3 hours in rodents and projected at 2 hours in humans) and produces deep but cyclic inhibition of MEK and ERK, leading to improved activity over current first-generation MEKi and potentially better safety and durability of response. Compound A may act as a dual-MEK inhibitor with a short half-life that displays a mechanism of action resulting in deep, more durable responses in RAS-mutant tumor models. On the basis of susceptibility to CRAF bypass and toxicity, the dynamic cyclic inhibition of MEK allows MAPK signaling to recover between dosing during cycling near-zero drug troughs when Compound A is administered once daily.

In contrast, first-generation MEK inhibitors, which include cobimetinib, binimetinib, and selumetinib, sustain suppression of MAPK pathway activity through chronic occupancy of MEK1 and MEK2 (MEK). At the same time, they display dose-limiting, class-effect toxicities and are sensitive to pathway reactivation events. Although second-generation MEKi, which includes VS-6766 (CH5126766), display mechanistic resistance to pathway reactivation, they also sustain suppression of MAPK pathway activity through chronic occupancy of MEK1 and MEK2 and, as such, share similar class-effect toxicities to first-generation MEKi. Selumetinib has a human plasma half-life of 5.3-7.2 h and a tmax of 1-1.5 h, which results in a sustained, non-zero drug trough with BID dosing (≤2.3 half-lives per BID cycle) and chronic ablation of MAPK signaling. Binimetinib has a shorter human plasma half-life of 3.5 h and tmax of 1.6 h but this still does not allow for a near-zero drug trough (≤3.4 half-lives per BID cycle). Conversely, cobimetinib has a long human plasma half-life of 44 h and tmax of 2.4 h (≤0.55 half-lives per cycle) while trametinib, with its long human plasma half-life of 96 hours has virtually no trough in exposure (≤0.25 half-lives per cycle).

In preclinical studies, Compound A inhibited MEK and ERK across a wide range of human and murine solid tumor models, including those with activating mutations in KRAS, NRAS, HRAS and BRAF. In addition, in head-to-head preclinical studies, Compound A was evaluated in murine-based KRAS, NRAS, and BRAF mutant solid tumor models representing lung, colon, pancreas and skin cancer, and observed tumor stasis or regression with insignificant body weight loss, or BWL, when compared to certain FDA-approved MEK inhibitors at reported human dose equivalent dose and schedules. For example, in head-to-head preclinical studies, Compound A was evaluated in murine-based KRAS, NRAS, and BRAF mutant solid tumor models representing lung (i.e., A549), colon (i.e., Colon-26), pancreas (i.e., MIA PaCa-2 and CAPAN-2) and skin cancer (i.e., A375 and SK-MEL-2), and observed superior antitumor activity with insignificant body weight loss, or BWL, when compared to certain FDA-approved MEK inhibitors and superior to non-inferior antitumor activity when compared to certain FDA-approved KRAS-G12C and BRAF inhibitors at reported human dose equivalent dose and schedules.

FIGS. 1A and 1B are a summary of results obtained from preclinical testing of Compound A in various xenograft tumor models, for pancreatic cancer (KRAS-G12C and KRAS-G12V), melanoma (NRAS-Q61R), non-small cell lung cancer (NSCLC) (KRAS-G12S), and colorectal cancer (KRAS-G12D). Compound A was well tolerated with a median body weight loss≤3-6% of baseline at top doses. Maximum effective dose range in mice (plasma t½=1.3 hours) is 100 mg/kg to 150 mg/kg BID po. CRC=colorectal cancer, NSCLC=non-small cell lung cancer; MEL—melanoma; PANC=pancreatic cancer. Tumor Growth Inhibition; TGI %=[1−(Ti−T₀)/(Ci−C₀)]×100; T=treatment groups; C=control groups. Capan-2 PANC model (0% TGI for sotorasib and adagrasib at top doses).

In some embodiments, methods of treating, modulating, preventing, reducing, or ameliorating a RAS-mutated cancer in a subject are provided. In some embodiments, the methods can comprise the administration of a therapeutically effective amount of a dual-MEK inhibitor inhibiting both the phosphorylation of mitogen-activated protein kinase kinase MEK1 and MEK2 (MEK) and the phosphorylation of mitogen-activated protein kinase (pERK), wherein the dual-MEK inhibitor is administered to the patient (a) in a dose amount that achieves a Cmax effective to inhibit pERK and MEK and (b) with an interval between consecutive doses that is at least about seven times the plasma half-life of the dual-MEK inhibitor. In some embodiments, the Cmax measured on the day of initial administration of the dual-MEK inhibitor may be at least 2,000 ng/mL. In some embodiments, the dual-MEK inhibitor can have at least about 1 micromolar dual-MEK inhibitor free fraction. In some embodiments, the Cmin, about 0.1 micromolar free fraction, of the dual-MEK inhibitor preferably occurs more than about 6 hours after the administration of the dual-MEK inhibitor. For example, the Cmin of about 0.1 micromolar free fraction occurs about 6-8 hours after the administration of the dual-MEK inhibitor, or later. In some embodiments, the interval between consecutive doses of the dual-MEK inhibitor that is about seven to eighteen times the plasma half-life of the dual-MEK inhibitor. In some embodiments, the dual-MEK inhibitor is Compound A or a pharmaceutically acceptable salt thereof.

In some embodiments, the cancer has a RAS mutation that is a KRAS G12D or G12V mutation or a NRAS Q61L or Q61K mutation. In some embodiments, the cancer has a KRAS G12D mutation. In some embodiments, the cancer has a KRAS G12V mutation. In some embodiments, the cancer has a NRAS Q61L mutation. In some embodiments, the cancer has a NRAS Q61K mutation.

In some embodiments, the cancer is selected from the group consisting of: NRAS-mutated melanoma, KRAS-mutated PDAC, KRAS-mutated NSCLC and KRAS-mutated, and APC-wildtype CRC.

In some embodiments, the cancer is melanoma. In some embodiments, the cancer is NRAS-mutated melanoma.

In some embodiments, the cancer is pancreatic ductal adenocarcinoma (PDAC). As described in Example 9, Preclinical mouse models in PDAC, non-small cell lung cancer, melanoma, and colorectal cancer demonstrated tumor reduction across all dose levels and in all models. Additionally, Compound A showed greater reductions in tumor volume in head-to-head comparisons with approved MEK inhibitors. In some embodiments, the cancer is KRAS-mutated PDAC.

In some embodiments, the cancer is lung cancer. In some embodiments, the cancer is KRAS-mutated NSCLC.

In some embodiments, the cancer is colorectal cancer (CRC). In some embodiments, the cancer is KRAS-mutated CRC. In some embodiments, the cancer is APC-wildtype CRC. In some embodiments, the cancer is pancreatic cancer with a KRAS G12C mutation. In some embodiments, the cancer is pancreatic cancer with a G12D mutation. In some embodiments, the cancer is pancreatic cancer with a G12V mutation. In some embodiments, the cancer is colorectal cancer with a NRAS Q61L mutation. In some embodiments, the cancer is colorectal cancer with a NRAS Q61K mutation. In some embodiments, the cancer is colorectal cancer with a KRAS G12D mutation. In some embodiments, the cancer is melanoma with a NRAS Q61R mutation. In some embodiments, the cancer is melanoma with a KRAS G12S mutation. In some embodiments, the cancer melanoma with a BRAF V600E mutation. In some embodiments, the cancer is non-small cell lung cancer (NSCLC) with a KRAS G12S mutation.

In some embodiments, the dual-MEK inhibitor is Compound A administered to the patient once daily in a dose amount of about 40 mg to treat a RAS mutated cancer. In some embodiments, the dual-MEK inhibitor is Compound A administered to the patient once daily in a dose amount of 60 mg to treat a RAS mutated cancer. In some embodiments, the dual-MEK inhibitor is Compound A administered to the patient once daily in a dose amount of about 80 mg to treat a RAS mutated cancer. In some embodiments, the dual-MEK inhibitor is Compound A administered to the patient once daily in a dose amount of about 100 mg to treat a RAS mutated cancer. In some embodiments, the dual-MEK inhibitor is Compound A administered to the patient once daily in a dose amount of about 120 mg to treat a RAS mutated cancer. In some embodiments, the dual-MEK inhibitor is Compound A administered to the patient once daily in a dose amount of about 140 mg to treat a RAS mutated cancer. In some embodiments, the dual-MEK inhibitor is Compound A administered to the patient once daily in a dose amount of about 160 mg to treat a RAS mutated cancer. In some embodiments, the dual-MEK inhibitor is Compound A administered to the patient once daily in a dose amount of about 180 mg to treat a RAS mutated cancer. In some embodiments, the dual-MEK inhibitor is Compound A administered to the patient once daily in a dose amount of about 200 mg to treat a RAS mutated cancer. In some embodiments, the dual-MEK inhibitor is Compound A administered to the patient once daily in a dose amount of about 240 mg to treat a RAS mutated cancer. In some embodiments, the dual-MEK inhibitor is Compound A administered to the patient once daily in a dose amount of about 260 mg to treat a RAS mutated cancer. In some embodiments, the dual-MEK inhibitor is Compound A administered to the patient once daily in a dose amount of about 280 mg to treat a RAS mutated cancer. In some embodiments, the dual-MEK inhibitor is Compound A administered to the patient once daily in a dose amount of about 300 mg to treat a RAS mutated cancer. In some embodiments, the dual-MEK inhibitor is Compound A administered to the patient once daily in a dose amount of about 320 mg to treat a RAS mutated cancer. In some embodiments, the dual-MEK inhibitor is Compound A administered to the patient once daily in a dose amount of about 340 mg to treat a RAS mutated cancer. In some embodiments, the dual-MEK inhibitor is Compound A administered to the patient once daily in a dose amount of about 360 mg to treat a RAS mutated cancer. In some embodiments, the dual-MEK inhibitor is Compound A administered to the patient once daily in a dose amount of about 380 mg to treat a RAS mutated cancer. In some embodiments, the dual-MEK inhibitor is Compound A administered to the patient once daily in a dose amount of about 400 mg to treat a RAS mutated cancer. In some embodiments, the dual-MEK inhibitor is Compound A administered to the patient once daily in a dose amount of about 420 mg to treat a RAS mutated cancer. In some embodiments, the dual-MEK inhibitor is Compound A administered to the patient once daily in a dose amount of about 440 mg to treat a RAS mutated cancer. In some embodiments, the dual-MEK inhibitor is Compound A administered to the patient once daily in a dose amount of about 460 mg to treat a RAS mutated cancer. In some embodiments, the dual-MEK inhibitor is Compound A administered to the patient once daily in a dose amount of about 480 mg to treat a RAS mutated cancer. In some embodiments, the dual-MEK inhibitor is Compound A administered to the patient once daily in a dose amount of about 500 mg to treat a RAS mutated cancer.

In some embodiments, the dual-MEK inhibitor is Compound A administered to the patient once daily in a dose of about 40 mg, 50 mg, 60 mg, 70 mg, 80 mg, 90 mg, 100 mg, 110 mg 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, or ranges including and/or spanning the aforementioned values. In some embodiments, the dual-MEK inhibitor is Compound A administered to the patient once daily in a dose of about 40 mg to about 500 mg to treat a RAS mutated cancer. In some embodiments, the dual-MEK inhibitor is Compound A administered to the patient once daily in a dose of about 40 mg to about 160 mg to treat a RAS mutated cancer. In some embodiments, the dual-MEK inhibitor is Compound A administered to the patient once daily in a dose of about 40 mg to about 80 mg to treat a RAS mutated cancer. In some embodiments, the dual-MEK inhibitor is Compound A administered to the patient once daily in a dose amount of 40 mg to about 320 mg to treat a RAS mutated cancer. In some embodiments, the dual-MEK inhibitor is Compound A administered to the patient once daily in a dose of about 80 to about 500 mg to treat a RAS mutated cancer. In some embodiments, the dual-MEK inhibitor is Compound A administered to the patient once daily in a dose of about 80 to about 320 mg to treat a RAS mutated cancer. In some embodiments, the dual-MEK inhibitor is Compound A administered to the patient once daily in a dose of about 80 to about 160 mg to treat a RAS mutated cancer. In some embodiments, the dual-MEK inhibitor is Compound A administered to the patient once daily in a dose amount of about 80 mg to about 120 mg to treat a RAS mutated cancer. In some embodiments, the dual-MEK inhibitor is Compound A administered to the patient once daily in a dose of about 100 to about 500 mg to treat a RAS mutated cancer. In some embodiments, the dual-MEK inhibitor is Compound A administered to the patient once daily in a dose of about 100 to about 320 mg to treat a RAS mutated cancer. In some embodiments, the dual-MEK inhibitor is Compound A administered to the patient once daily in a dose of about 100 to about 160 mg to treat a RAS mutated cancer. In some embodiments, the dual-MEK inhibitor is Compound A administered to the patient once daily in a dose amount of about 100 mg to about 120 mg to treat a RAS mutated cancer. In some embodiments, the dual-MEK inhibitor is Compound A administered to the patient once daily in a dose of about 120 to about 500 mg to treat a RAS mutated cancer. In some embodiments, the dual-MEK inhibitor is Compound A administered to the patient once daily in a dose of about 120 to about 320 mg to treat a RAS mutated cancer. In some embodiments, the dual-MEK inhibitor is Compound A administered to the patient once daily in a dose of about 120 to about 160 mg to treat a RAS mutated cancer. In some embodiments, the dual-MEK inhibitor is Compound A administered to the patient once daily in a dose amount of about 120 mg to about 140 mg to treat a RAS mutated cancer. In some embodiments, the dual-MEK inhibitor is Compound A administered to the patient once daily in a dose of about 160 to about 500 mg to treat a RAS mutated cancer. In some embodiments, the dual-MEK inhibitor is Compound A administered to the patient once daily in a dose amount of about 160 mg to about 320 mg to treat a RAS mutated cancer. In some embodiments, the dual-MEK inhibitor is Compound A administered to the patient once daily in a dose of about 160 to about 240 mg to treat a RAS mutated cancer. In some embodiments, the dual-MEK inhibitor is Compound A administered to the patient once daily in a dose of about 160 to about 180 mg to treat a RAS mutated cancer.

In some embodiments, compound A may be formulated as a pharmaceutical composition using a pharmaceutically acceptable excipients and administered by a variety of routes. In some embodiments, such compositions are for oral administration. Such pharmaceutical compositions and processes for preparing them are well known in the art. (See, e.g., Remington: The Science and Practice of Pharmacy, L. V. Allen, Editor, 22^{nd} Edition, Pharmaceutical Press, 2012).

In a particular embodiment, the pharmaceutical composition comprises 4-((dimethylamino)methyl)-3-(2-fluoro-3-((N-methylsulfamoyl)amino)benzyl)-2-oxo-2H-chromen-7-yl dimethylcarbamate or a pharmaceutically acceptable salt or hydrate thereof, and a pharmaceutically acceptable carrier.

In some embodiments, to prepare a pharmaceutical composition for oral delivery, 0.1 mg to 120 mg of a compound of an embodiment is mixed with 750 mg of starch. The mixture is incorporated into an oral dosage unit, such as a hard gelatin capsule, or 0.1 mg to 120 mg of compound is granulated with binder solution such as starch solution along with suitable diluents such as microcrystalline cellulose or like, disintegrants such as croscarmellose sodium, dry the resultant mixture and add lubricant and compress into tablet which is suitable for oral administration.

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g., intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. In some embodiments, the pharmaceutical composition can be a medicament.

In some embodiments, pharmaceutical compositions comprising Compound A or pharmaceutically acceptable salts thereof can be formulated for oral administration. For example, a compound provided herein can be combined with suitable compendial excipients to form an oral unit dosage form, such as a capsule or tablet, containing a target dose of Compound A. The drug product can be prepared by first manufacturing Compound A as an active pharmaceutical ingredient (API), followed by roller compaction/milling with intragranular excipients and blending with extra granular excipients. A Drug Product can contain Compound A as the API and excipient components in a tablet in a desired dosage strength of Compound A. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include Compound A as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this disclosure, active compounds can be given per se or as a pharmaceutical composition containing, for example, 0.1 to 99.5% (or, for example, 0.5 to 90%) of an active ingredient in combination with a pharmaceutically acceptable carrier.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present disclosure may contain conventional pharmaceutical carriers and/or auxiliary agents. In some embodiments, he pharmaceutical compositions according to the present disclosure may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

In some embodiments, an oral dosage form of Compound A is provided, such as the oral dosage form disclosed in Example 11. In some embodiments, an oral dosage form such as a tablet or capsule can contain Compound A or a pharmaceutically acceptable salt thereof as the API and one or more excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg to 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg, 120 mg or 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former and one or more extra granulation excipients such as additional Compound A API granulate and a lubricant or film forming agent.

In some embodiments, an oral dosage form such as a tablet or capsule can contain 20 mg, 120 mg or 160 mg of Compound A or a pharmaceutically acceptable salt thereof as the API and one or more intra-granulation excipients such as microcrystalline cellulose, crospovidone Type A, and hydroxypropyl cellulose, and one or more extra-granulation excipients such as API granulate of Compound A and magnesium stearate. In some embodiments, an oral dosage form such as a tablet or capsule can contain about 20% w/w of Compound A or a pharmaceutically acceptable salt thereof as the API and about 60-70% w/w microcrystalline cellulose, about 5% w/w crospovidone Type A, and about 10% w/w hydroxypropyl cellulose, and one or more extra-granulation excipients such as API granulate of Compound A (95-99.5% w/w) and magnesium stearate (0.5-5% w/w).

Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In certain aspects, therapeutic methods or uses are provided herein for treating, preventing, or ameliorating a disease or condition in a subject. Some embodiments relate to the use of a compound or composition described herein to treat, prevent, or ameliorate cancer associated with an oncogenic RAS mutation. Some embodiments relate to the use of a compound or composition as described herein to treat cancer characterized by expressing a mutant form of RAS protein in a subject. Some embodiments relate to the use of a compound or composition as described herein to inhibit the proliferation of a cell having a RAS mutation. Some embodiments relate to the use of a compound or composition as described herein to induce apoptosis in a cell having a RAS mutation. Some embodiments relate to the use of a compound or composition as described herein to inhibit the progression of cancer that is characterized by expressing a mutant form of RAS protein in a subject. In some embodiments, the RAS protein is a KRAS protein. In some embodiments, the RAS protein is an NRAS protein. In some embodiments, the RAS protein is an HRAS protein.

Some embodiments relate to a method of therapeutically treating, modulating, preventing, reducing, or ameliorating a cancer characterized by expressing a mutant form of a RAS protein. In some embodiments, the method includes administering to a subject in need thereof a compound of Formula (IIb):

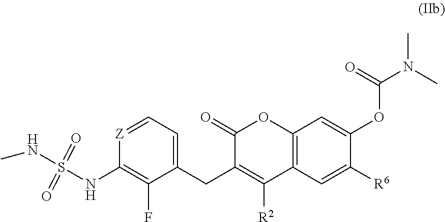

or a pharmaceutically acceptable salt thereof, wherein Z is C, R² is L, L is —Z₁-Z₂, Z₁ is —CH₂—, Z₂ is —NR⁵R^{5′}, each of R⁵ and R^{5′} are each independently methyl, wherein, the RAS protein is a mutant form of a HRAS protein, a KRAS protein, or a NRAS protein.

Some embodiments provide a compound of Formula (II):

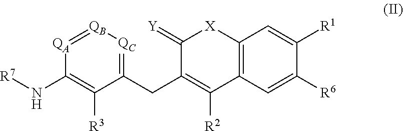

wherein R⁷ is methyl; each of Q_{A}, Q_{B}, and Q_{C} are each independently selected from C or N; X and Y are each O; R³ is F; R² is —CH₂—N(CH₃)₂; R⁶ is halo or H and R¹ is —O—C(O)—N(CH₃)₂. In some embodiments, Formula (II) is a pharmaceutically acceptable salt as described herein. In some embodiments, Q_{A} is N, Q_{B} is C, and Q_{C} is C. In some embodiments, Q_{A} is C, Q_{B} is C, and Q_{C} is C.

In some embodiments, Formula (II) is represented by Formula (IIa), Formula (IIb), Formula (IIc), or Formula (IId):

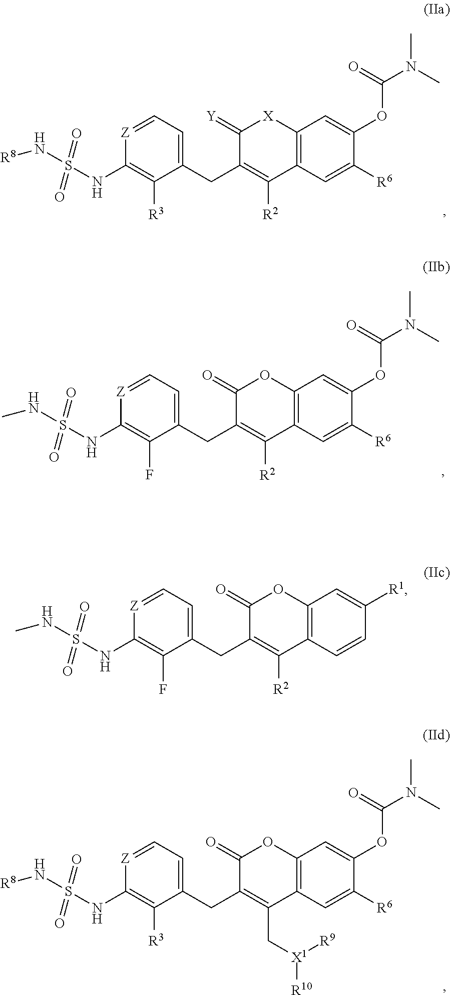

wherein R⁸ is methyl; Z is C or N; X and Y are each O; R³ is F; R² is —CH₂—N(CH₃)₂; X¹ is N and R⁹ and R¹⁰ are both methyl; R⁶ is H and R¹ is —O—C(O)—N(CH₃)₂. In some embodiments, Formula (IIa), Formula (IIb), Formula (IIe), or Formula (IId) may be a pharmaceutically acceptable salt as described herein. In some embodiments, Formula (IIa), (IIb), or (IIc) R⁵ and R^{5′} are each independently methyl, ethyl, propyl, iso-propyl, n-butyl, iso-butyl, sec-butyl, or t-butyl; and R₆ is H. In some embodiments, Formula (IIa), Formula (IIb), Formula (IIc), or Formula (IId) Z is N.

In some embodiments, Compound A may also be designated Compound 9 in Table 1:

TABLE 1

| No. | Structure |
|---|---|
| 9 | 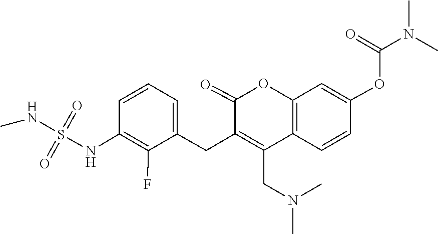 |

In some embodiments, the HRAS protein, the KRAS protein, or the NRAS protein comprises a mutation at one or more positions selected from codons 12, 13, 59, 61, and 146. In some embodiments, the RAS protein is a mutant form of the HRAS protein, the KRAS protein, or the NRAS protein is comprising one or more amino acid substitutions selected from the group consisting of G12C, G12R, G12D, G12V, G12F, G12L, G12N, G13C, G13R, G13S, G13A, G13V, G13P, S17G, P34S, A59E, A59G, A59T, Q61K, Q61L, Q61R, Q61H, K117N, A146P, A146T, A146V, and D153V. In some embodiments, the RAS protein is a mutant form of the KRAS protein is comprising one or more amino acid substitutions selected from the group consisting of A146T, G12C, G12D, G12R, and G12V. In some embodiments, the RAS protein is a mutant form of the NRAS protein is comprising one or more amino acid substitutions selected from the group consisting of Q61K, Q61L, and Q61R. In some embodiments, the RAS protein is a mutant form of the HRAS protein is G13D. In some embodiments, the cancer is selected from colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, fibrosarcoma, neuroblastoma, glioblastoma, brain metastases, salivary gland carcinoma, thyroid cancer, brain cancer, lymphoma, myeloma, and head and neck cancer. In some embodiments, the cancer is selected from pancreatic cancer, melanoma, colorectal cancer, lung cancer, thyroid cancer, breast cancer, gastric cancer, ovarian cancer, prostate cancer, fibrosarcoma, liver cancer, and neuroblastoma. In some embodiments, Formula (II), (IIa), Formula (IIb), Formula (IIc), R⁵ and R^{5′} are each independently methyl, ethyl, propyl, iso-propyl, n-butyl, iso-butyl, sec-butyl, or t-butyl; and R₆ is H. In some embodiments, Formula (IIa), Formula (IIb), Formula (IIc), or Formula (IId) Z is C. In some embodiments, Formula (IIa), Formula (IIb), Formula (IIc), or Formula (IId) Z is N. In some embodiments, the compound of Formula (IIa), (IIb), (IIc), or (IId) is a compound selected from Table 1.

Some embodiments relate to a method of treating a cancer characterized by expressing a mutant form of a RAS protein, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of treating a cancer associated with an oncogenic RAS mutation, comprising administering an effective amount of a compound of Formula (II), (IIa), (IIb), (IIc), (IId), or a pharmaceutically acceptable salt thereof, or a pharmaceutical composition to a subject in need thereof. In some embodiments, the mutant form of the RAS protein is a mutant form of a KRAS, NRAS, or HRAS protein. In some embodiments, the mutant form of the RAS is a mutant form of the KRAS protein. In some embodiments, the mutant form of the RAS is a mutant form of the NRAS protein. In some embodiments, the RAS gene mutation is KRAS gene mutation. In some embodiments, the RAS gene mutation is NRAS gene mutation.

Some embodiments relate to a method of treating a cancer characterized by expressing a mutant form of a KRAS protein, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of treating a cancer associated with an oncogenic KRAS mutation, comprising administering an effective amount of a compound of Formula (II), (IIa), (IIb), (IIc), (IId), or a pharmaceutically acceptable salt thereof, or a pharmaceutical composition to a subject in need thereof. In some embodiments, the KRAS comprises mutation at one or more positions selected from codons 12, 13, 59, and 61. In some embodiments, the mutant form of the KRAS protein has mutation at one or more amino acid positions selected from G12, G13, S17, P34, A59, and Q61. In some embodiments, the mutant form of the KRAS protein has one or more amino acid substitutions selected from the group consisting of G12C, G12S, G12R, G12F, G12L, G12N, G12A, G12D, G12V, G13C, G13S, G13D, G13V, G13P, S17G, P34S, A59E, A59G, A59T, Q61K, Q61L, Q61R, and Q61H. In some embodiments, the mutant form of the KRAS protein has mutation at one or more amino acid positions selected from G12, G13, A59, Q61, K117 and A146. In some embodiments, the mutant form of the KRAS protein has one or more amino acid substitutions selected from the group consisting of G12C, G12R, G12S, G12A, G12D, G12V, G13C, G13R, G13S, G13A, G13D, G13V, A59E, A59G, A59T, Q61K, Q61L, Q61R, Q61H, K117N, K117R, K117E, A146P, A146T and A146V. In some embodiments, the mutant form of the KRAS protein has mutation at one or more amino acid positions selected from of G12, G13, A59 and Q61. In some embodiments, the mutant form of the KRAS protein has one or more amino acid substitutions selected from the group consisting of G12C, G12R, G12S, G12A, G12D, G12V, G13C, G13R, G13S, G13A, G13D, A59E, A59G, A59T, Q61K, Q61L, Q61R and Q61H. In some embodiments, the mutant form of the KRAS protein has mutation at one or more amino acid positions selected from G12, G13, and D153. In some embodiments, the mutant form of the KRAS protein has one or more amino acid substitutions selected from the group consisting of G12A, G12C, G12D, G12V, G12S, G13D, and D153V. In some embodiments, the mutant form of the KRAS protein has one or more amino acid substitutions selected from G12C, G12S, and D153V. In some embodiments, the mutant form of the KRAS protein is comprising one or more amino acid substitutions selected from the group consisting of A146T, G12C, G12D, G12R, and G12V.

Some embodiments relate to a method of treating a cancer characterized by expressing a mutant form of a NRAS protein, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of treating a cancer associated with an oncogenic NRAS mutation, comprising administering an effective amount of a compound of Formula (II), (IIa), (IIb), (IIc), (IId), or a pharmaceutically acceptable salt thereof, or a pharmaceutical composition to a subject in need thereof. In some embodiments, the NRAS comprises mutation at one or more positions selected from codons 12, 13, 59, 61, and 146. In some embodiments, the mutant form of the NRAS protein has mutation at one or more amino acid positions selected from G12, G13, A59, Q61, K117 and A146. In some embodiments, the mutant form of the NRAS protein has one or more amino acid substitutions selected from the group consisting of G12C, G12R, G12S, G12A, G12D, G12V, G13C, G13R, G13S, G13A, G13D, G13V, A59D, A59T, Q61K, Q61L, Q61R, Q61H, K117N, K117R, K117E, A146P, A146T and A146V. In some embodiments, the mutant form of the NRAS protein has mutation at one or more amino acid positions at Q61 or A146. In some embodiments, the mutant form of the NRAS protein has one or more amino acid substitutions selected from Q61K, Q61H, Q61R, Q61L, Q61N, Q61E, Q61P, A146T, A146P, A146V, and any combinations thereof. In some embodiments, the mutant form of the NRAS protein has one or more amino acid substitutions selected from Q61H, Q61R, Q61L, and any combinations thereof. In some embodiments, the mutant form of the NRAS protein is comprising one or more amino acid substitutions selected from the group consisting of Q61K, Q61L, and Q61R.

Some embodiments relate to a method of treating a cancer characterized by expressing a mutant form of a HRAS protein, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of treating a cancer associated with an oncogenic HRAS mutation, comprising administering an effective amount of a compound of Formula (II), (IIa), (IIb), (IIc), (IId), or a pharmaceutically acceptable salt thereof, or a pharmaceutical composition to a subject in need thereof. In some embodiments, the HRAS comprises mutation at one or more positions selected from codons 12, 13, 59, 61, and 146. In some embodiments, the mutant form of the HRAS protein has mutation at one or more amino acid positions selected from G12, G13, A59, Q61, K117 and A146. In some embodiments, the mutant form of the HRAS protein has one or more amino acid substitutions selected from the group consisting of G12C, G12R, G12S, G12A, G12D, G12V, G13C, G13R, G13S, G13A, G13D, G13V, A59D, A59T, Q61K, Q61L, Q61R, Q61H, K117N, K117R, K117E, A146P, A146T and A146V. In some embodiments, the mutant form of the HRAS protein has mutation at one or more amino acid positions at Q61 or A146. In some embodiments, the mutant form of the HRAS protein has one or more amino acid substitutions selected from Q61K, Q61H, Q61R, Q61L, Q61N, Q61E, Q61P, A146T, A146P, A146V, and any combinations thereof. In some embodiments, the mutant form of the HRAS protein has one or more amino acid substitutions selected from Q61H, Q61R, Q61L, and any combinations thereof. In some embodiments, the mutant form of the HRAS protein is G13D.

Some embodiments relate to a method of inhibiting proliferation of a cell having a RAS mutation, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of inducing apoptosis in a cell having a RAS mutation, comprising contacting the cell with an effective amount of a compound of Formula (II), (IIa), (IIb), (IIc), (IId), or a pharmaceutically acceptable salt thereof, or a pharmaceutical composition. In some embodiments, the contacting includes administering an effective amount of a compound of Formula (II), (IIa), (IIb), (IIc), (IId), or a pharmaceutically acceptable salt thereof, or a pharmaceutical composition to a subject in need thereof. Some embodiments relate to a method of inhibiting progression of a cancer characterized by expressing a mutant form of RAS in a subject, comprising contacting the cell with an effective amount of a compound of Formula (II), (IIa), (IIb), (IIc), (IId), or a pharmaceutically acceptable salt thereof, or a pharmaceutical composition. In some embodiments, the contacting includes administering an effective amount of a compound of Formula (II), (IIa), (IIb), (IIc), (IId), or a pharmaceutically acceptable salt thereof, or a pharmaceutical composition to a subject in need thereof.

Some embodiments relate to a method of reducing the level of activity of a RAS protein, the method including contacting the RAS protein with a compound of Formula (II), (IIa), (IIb), (IIc), (IId), or a pharmaceutically acceptable salt thereof, or a pharmaceutical composition as described herein. In some embodiments, the activity of the RAS protein is its GTPase activity, nucleotide exchange, differential GDP or GTP binding, effector protein binding, effector protein activation, guanine exchange factor (FEF) binding, GEF-facilitated nucleotide exchange, phosphate release, nucleotide release, nucleotide binding, RAS subcellular localization, RAS post-translational processing, RAS-posttranslational modifications, prenylation, or a GTP bound RAS signaling pathway. Some embodiments relate to a method of modulating the level of activity of a RAS protein, the method including contacting the RAS protein with a compound of Formula (II), (IIa), (IIb), (IIc), (IId), or a pharmaceutically acceptable salt thereof, or a pharmaceutical composition as described herein. In some embodiments, modulating is increasing the activity of a RAS protein. In some embodiments, modulating is decreasing the activity of a RAS protein. In some embodiments, the compound modulates the activity of KRAS at least two-fold more than it modulates the activity of HRAS. In some embodiments, the compound modulates the activity of KRAS at least five-fold more than it modulates the activity of HRAS.

Some embodiments relate to a method of inhibiting proliferation of a cell having a RAS mutation, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of inducing apoptosis in a cell having a RAS mutation, comprising contacting the cell with an effective amount of a compound of Formula (II), (IIa), (IIb), (IIc), (IId), or a pharmaceutically acceptable salt thereof, or a pharmaceutical composition. Some embodiments relate to a method of inhibiting progression of a cancer characterized by expressing a mutant form of RAS in a subject, comprising contacting the cell with an effective amount of a compound of Formula (II), (IIa), (IIb), (IIc), (IId), or a pharmaceutically acceptable salt thereof, or a pharmaceutical composition.

Some embodiments relate to a method of inhibiting proliferation of a cell having a NRAS mutation, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of inducing apoptosis in a cell having a NRAS mutation, comprising contacting the cell with an effective amount of a compound of Formula (II), (IIa), (IIb), (IIc), (IId), or a pharmaceutically acceptable salt thereof, or a pharmaceutical composition. Some embodiments relate to a method of inhibiting progression of a cancer characterized by expressing a mutant form of NRAS in a subject, comprising contacting the cell with an effective amount of a compound of Formula (II), (IIa), (IIb), (IIc), (IId), or a pharmaceutically acceptable salt thereof, or a pharmaceutical composition.

Some embodiments relate to a method of inhibiting proliferation of a cell having a HRAS mutation, comprising administering an effective amount of a compound of Formula (II), (IIa), (IIb), (IIc), (IId), or a pharmaceutically acceptable salt thereof, or a pharmaceutical composition to a subject in need thereof. Some embodiments relate to a method of inducing apoptosis in a cell having a HRAS mutation, comprising contacting the cell with an effective amount of a compound of Formula (II), (IIa), (IIb), (IIc), (IId), or a pharmaceutically acceptable salt thereof, or a pharmaceutical composition. Some embodiments relate to a method of inhibiting progression of a cancer characterized by expressing a mutant form of HRAS in a subject, comprising contacting the cell with an effective amount of a compound of Formula (II), (IIa), (IIb), (IIc), (IId), or a pharmaceutically acceptable salt thereof, or a pharmaceutical composition.

Some methods described herein can further include identifying a patient having a cancer characterized by expressing a mutant type of RAS protein. In some embodiments, identifying a patient can include determining whether the patient has a RAS mutation. Some embodiments relate to a method for treating cancer in a patient identified as having a RAS mutation, the method comprising administering to the patient an effective amount of a compound of Formula (II), (IIa), (IIb), (IIc), (IId), or a pharmaceutically acceptable salt thereof, wherein the patient has been identified by (i) collecting a sample from the patient; (ii) isolating DNA from the sample; (iii) amplifying a RAS gene or fragment thereof in the isolated DNA; and (iv) detecting whether there is a mutation in the amplified RAS gene, thereby determining whether the patient has a cancer characterized by a RAS mutation. Examples of methods for detecting RAS mutation include but are not limited to Amplified Refractory Mutation System (ARMS) PCR, BEAMing assays, digital PCR, and other suitable primers, and probes for sequencing or PCR.

Some methods described herein can further include identifying a patient having a cancer characterized by expressing a mutant type of KRAS. In some embodiments, identifying a patient can include determining whether the patient has a KRAS mutation. Some embodiments relate to a method for treating cancer in a patient identified as having a KRAS mutation, the method comprising administering to the patient an effective amount of a compound of Formula (II), (IIa), (IIb), (IIc), (IId), or a pharmaceutically acceptable salt thereof, wherein the patient has been identified by (i) collecting a sample from the patient; (ii) isolating DNA from the sample; (iii) amplifying a KRAS gene or fragment thereof in the isolated DNA; and (iv) detecting whether there is a mutation in the amplified KRAS gene, thereby determining whether the patient has a cancer characterized by a KRAS mutation.

Some methods described herein can further include identifying a patient having a cancer characterized by expressing a mutant type of NRAS protein. In some embodiments, identifying a patient can include determining whether the patient has a NRAS mutation. Some embodiments relate to a method for treating cancer in a patient identified as having a NRAS mutation, the method comprising administering to the patient an effective amount of a compound of Formula (II), (IIa), (IIb), (IIc), (IId), or a pharmaceutically acceptable salt thereof, wherein the patient has been identified by (i) collecting a sample from the patient; (ii) isolating DNA from the sample; (iii) amplifying a NRAS gene or fragment thereof in the isolated DNA; and (iv) detecting whether there is a mutation in the amplified NRAS gene, thereby determining whether the patient has a cancer characterized by a NRAS mutation.

Some methods described herein can further include testing for a NRAS mutation. In some embodiments, NRAS mutations, e.g., at codons 12, 13, 59, 61 and/or 146, may be detected from pathology samples taken from the patient. In some embodiments of such testing, DNA is removed from the sample and tested against labeled oligonucleotide probes using PCR to amplify the targeted mutated DNA to permit detection. Several commercially available kits (see Dxs Diagnostic Innovations, Applied Biosystems, and Quest diagnostics), primers and probes for sequencing or PCR can be designed based on the codon mutations of NRAS.

Some methods described herein can further include identifying a patient having a cancer characterized by expressing a mutant type of HRAS protein. In some embodiments, identifying a patient can include determining whether the patient has a HRAS mutation. Some embodiments relate to a method for treating cancer in a patient identified as having a HRAS mutation, the method comprising administering to the patient an effective amount of a compound of Formula (II), (IIa), (IIb), (IIc), (IId), or a pharmaceutically acceptable salt thereof, wherein the patient has been identified by (i) collecting a sample from the patient; (ii) isolating DNA from the sample; (iii) amplifying a HRAS gene or fragment thereof in the isolated DNA; and (iv) detecting whether there is a mutation in the amplified HRAS gene, thereby determining whether the patient has a cancer characterized by a HRAS mutation.

In some embodiments, the cancer with a RAS mutation is selected from the group consisting of colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, glioblastoma, brain metastases, salivary gland carcinoma, thyroid cancer, brain cancer, lymphoma, myeloma, and head and neck cancer. In some embodiments, the cancer is selected from squamous cell cancer, small-cell lung cancer, non-small cell lung cancer, adenocarcinoma of the lung, squamous carcinoma of the lung, hepatocellular carcinoma, colon cancer, endometrial carcinoma, and hepatocellular carcinoma. In some embodiments, the cancer is selected from colorectal cancer, prostate cancer, breast cancer, lung cancer, endometrial cancer, multiple myeloma, pancreatic, renal cancer, and glioblastoma. In some embodiments, the cancer is selected from pancreatic cancer, melanoma, colorectal cancer, lung cancer, thyroid cancer, breast cancer, gastric cancer, ovarian cancer, prostate cancer, fibrosarcoma, liver cancer, and neuroblastoma.

### Detecting RAS-Mutated Cancer

In some embodiments, patients are selected for treatment with Compound A based on the presence of RAS mutation such as a KRAS G12C mutation in plasma or tumor specimens. If no mutation is detected in a plasma specimen, test tumor tissue. Additional information on FDA-approved tests for the detection of a KRAS G12C mutation is available at: https://www.fda.gov/CompanionDiagnostics. FDA approved diagnostics for identifying patients with a RAS-mutated cancer include the companion diagnostics provided in Table 2 below.

TABLE 2

| | Indication | | |
|---|---|---|---|
| Diagnostic Name | Sample Type | Biomarker | Biomarker(s) (Details) |
| Agilent Resolution | Non-Small Cell | KRAS | KRAS G12C |
| ctDx FIRST assay | Lung Cancer | | |
| (Resolution | (NSCLC) - | | |
| Bioscience, Inc.) | Plasma | | |
| cobas KRAS | Colorectal | KRAS | Mutations in codons 12 and 13 |
| Mutation Test (Roche | Cancer - Tissue | | of KRAS gene |
| Molecular Systems, | | | |
| Inc.) | | | |
| cobas KRAS | Colorectal | KRAS | Mutations in codons 12 and 13 |
| Mutation Test (Roche | Cancer - Tissue | | of KRAS gene |
| Molecular Systems, | | | |
| Inc.) | | | |
| CRCDx RAS | Colorectal | KRAS and | KRAS wild-type biomarkers (the |
| Mutation Detection | Cancer (CRC) - | NRAS | absence of mutations in exons 2, |
| Assay Kit (EntroGen, | Tissue | | 3, or 4) and NRAS wild-type |
| Inc.) | | | biomarkers (the absence of |
| | | | mutations in exons 2, 3, or 4) |
| FoundationOne CDx | Colorectal | KRAS | KRAS wild-type (absence of |
| (Foundation | Cancer - Tissue | | mutations in codons 12 and 13) |
| Medicine, Inc.) | | | |
| Diagnostic Name | Indication | Biomarker | Biomarker(s) (Details) |
| | Sample Type | | |
| FoundationOne CDx | Colorectal | KRAS and | KRAS wild-type (absence of |
| (Foundation | Cancer - Tissue | NRAS | mutations in exons 2, 3, and 4) |
| Medicine, Inc.) | | | and NRAS wild type (absence of |
| | | | mutations in exons 2, 3, and 4) |
| Guardant360 CDx | Non-Small Cell | KRAS | G12C |
| (Guardant Health, | Lung Cancer | | |
| Inc.) | (NSCLC) - | | |
| | Plasma | | |
| ONCO/Reveal Dx | Colorectal | KRAS | KRAS wild-type (absence of |
| Lung & Colon Cancer | Cancer - Tissue | | mutations in codons 12 and 13) |
| Assay (O/RDx- | | | |
| LCCA) (Pillar | | | |
| Biosciences, Inc.) | | | |
| ONCO/Reveal Dx | Colorectal | KRAS | KRAS wild-type (absence of |
| Lung & Colon Cancer | Cancer - Tissue | | mutations in codons 12 and 13) |
| Assay (O/RDX- | | | |
| LCCA) (Pillar | | | |
| Biosciences, Inc.) | | | |
| therascreen KRAS | Colorectal | KRAS | G12A, G12D, G12R, G12C, |
| RGQ PCR Kit | Cancer - Tissue | | G12S, G12V, G13D |
| (Qiagen Manchester, | | | |
| Ltd.) | | | |
| therascreen KRAS | Non-Small Cell | KRAS | G12C |
| RGQ PCR Kit | Lung Cancer | | |
| (Qiagen Manchester, | (NSCLC) - | | |
| Ltd.) | Tissue | | |
| therascreen KRAS | Colorectal | KRAS | G12A, G12D, G12R, G12C, |
| RGQ PCR Kit | Cancer - Tissue | | G12S, G12V, G13D |
| (Qiagen Manchester, | | | |
| Ltd.) | | | |
| therascreen KRAS | Colorectal | KRAS | KRAS wild-type (absence of |
| RGQ PCR Kit | Cancer - Tissue | | mutations in codons 12 and 13) |
| (Qiagen Manchester, | | | |
| Ltd.) | | | |
| therascreen KRAS | Non-Small Cell | KRAS | KRAS G12C |
| RGQ PCR Kit | Lung Cancer | | |
| (Qiagen Manchester, | (NSCLC) - | | |
| Ltd.) | Tissue | | |
| xT CDx (Tempus | Colorectal | KRAS | KRAS wild-type (absence of |
| Labs, Inc.) | Cancer (CRC) - | | mutations in codons 12 or 13) |
| | Tissue | | |
| | (Matching | | |
| | Blood/Saliva) | | |
| xT CDx (Tempus | Colorectal | KRAS and | KRAS wild-type (absence of |
| Labs, Inc.) | Cancer (CRC) - | NRAS | mutations in exons 2, 3, or 4) |
| | Tissue | | and NRAS wild-type (absence of |
| | (Matching | | mutations in exons 2, 3, or 4) |
| | Blood/Saliva) | | |

In some embodiments, RAS mutations can be detected by next generation sequencing (NGS) techniques. For example, molecular diagnostics of HRAS/KRAS and NRAS targeted gene panels can be obtained by NGS techniques. Next generation sequencing of amplification products can be performed on the Ion Torrent Personal Genome Machine and analyzed with the Torrent Suite Software. Reference DNA sequences used for these genes can be found at https://www.ncbi.nlm.nih.gov/refseq/rsg/. This mutation panel is designed to detect targeted mutations only. Confirmation of these mutations is performed by traditional or real time PCR followed by Sanger sequencing, fluorescent melting curve analysis and/or pyrosequencing (CPT codes: 81210 (BRAF), 81275 (KRAS codons 12 and 13 in exon 2), 81276 (KRAS codon 61 in exon 3), 81403 (HRAS), 81311 (NRAS)).

### Definitions

The term “pharmaceutical composition,” as used herein, refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier,” as used herein, refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent,” as used herein, refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable,” as used herein, refers to a carrier or diluent that does not abrogate the biological activity and properties of the compound.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine, lysine, and the like.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

### ADDITIONAL EMBODIMENTS

In some embodiments, one or more of following methods are also provided.
A-1. A method of therapeutically treating a cancer characterized by expressing a mutant form of a RAS protein, the method comprising administering to a subject in need thereof a compound of Formula (IIb):

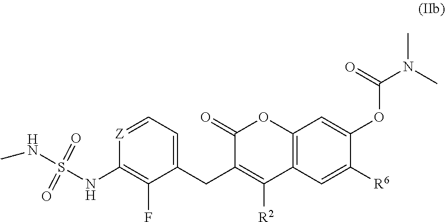

or a pharmaceutically acceptable salt thereof, wherein:
Z is C;
R₂ is L;
L is —Z₁-Z₂;
Z₁ is —CH₂—;
Z₂ is —NR⁵R^{5′},
each of R⁵ and R^{5′} are each independently C₁ to C₆ alkyl, and
R⁶ is H, deuterium, halogen, or C₁ to C₆ alkyl,
wherein, the RAS protein is a mutant form of a HRAS protein, a KRAS protein, or a NRAS protein.
A-2. The method of embodiment A-1, wherein the HRAS protein, the KRAS protein, or the NRAS protein comprises a mutation at one or more positions selected from codons 12, 13, 59, 61, and 146.
A-3. The method of embodiment A-1 or A-2, wherein the RAS protein is a mutant form of the HRAS protein, the KRAS protein, or the NRAS protein is comprising one or more amino acid substitutions selected from the group consisting of G12C, G12R, G12D, G12V, G12F, G12L, G12N, G13C, G13R, G13S, G13A, G13V, G13P, S17G, P34S, A59E, A59G, A59T, Q61K, Q61L, Q61R, Q61H, K117N, A146P, A146T, A146V, and D153V.
A-4. The method of embodiment A-3, wherein the RAS protein is a mutant form of the KRAS protein is comprising one or more amino acid substitutions selected from the group consisting of A146T, G12C, G12D, G12R, and G12V.
A-5. The method of embodiment A-3, wherein the RAS protein is a mutant form of the NRAS protein is comprising one or more amino acid substitutions selected from the group consisting of Q61K, Q61L, and Q61R.
A-6. The method of embodiment A-3, wherein the RAS protein is a mutant form of the HRAS protein is G13D.
A-7. The method of any one of embodiments A-1 to A-6, wherein the cancer is selected from colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, fibrosarcoma, neuroblastoma, glioblastoma, brain metastases, salivary gland carcinoma, thyroid cancer, brain cancer, lymphoma, myeloma, and head and neck cancer.
A-8. The method of embodiment A-7, wherein the cancer is selected from pancreatic cancer, melanoma, colorectal cancer, lung cancer, thyroid cancer, breast cancer, gastric cancer, ovarian cancer, prostate cancer, fibrosarcoma, liver cancer, and neuroblastoma.
A-9. The method of any one of embodiments A-1 to A-8, wherein, R5 and R5′ are each independently methyl, ethyl, propyl, iso-propyl, n-butyl, iso-butyl, sec-butyl, or t-butyl; and R6 is H.
A-10. The method of any one of embodiments A-1 to A-9, wherein the compound is:

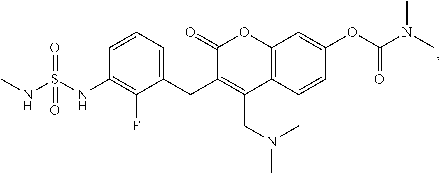

or a pharmaceutically acceptable salt thereof.
B-1. A method of treating a patient with a RAS-mutated cancer, comprising administering a therapeutically effective amount of a dual-MEK inhibitor inhibiting both phosphorylation of mitogen-activated protein kinase kinase 1 and 2 (pMEK) and phosphorylation of mitogen-activated protein kinase 1 and 2 (pERK), wherein the dual-MEK inhibitor is administered to the patient (a) in a dose amount that achieves a Cmax effective to inhibit pERK and pMEK and (b) with an interval between consecutive doses that is at least about seven times the plasma half-life of the dual-MEK inhibitor.
B-2. The method of embodiment B-1, wherein the Cmax measured on day 1 of administering the dual-MEK inhibitor is at least 2,000 ng/mL.
B-3. The method of embodiment B-2, wherein the dual-MEK inhibitor has at least about 1 micromolar dual-MEK inhibitor free fraction.
B-4. The method of any one of embodiments B-1 to B-3, wherein a Cmin of over about 0.1 micromolar occurs longer than about 6 hours after administering of the dual-MEK inhibitor in a free fraction form.
B-5. The method of any one of embodiments B-1 to B-3, wherein a Cmin of over about 0.1 micromolar occurs longer than about 6-8 hours after administering of the dual-MEK inhibitor in a free fraction form.
B-6. The method of any one of embodiments B-1 to B-5, wherein the interval between consecutive doses of the dual-MEK inhibitor is about seven to eight times the plasma half-life of the dual-MEK inhibitor.
B-7. The method of any one of embodiments B-1 to B-6, wherein the dual-MEK inhibitor is Compound A,

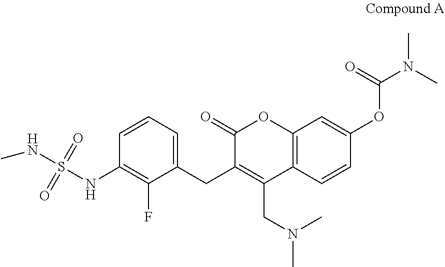

or a pharmaceutically acceptable salt thereof.
B-8. The method of any one of embodiments B-1 to B-7, wherein the RAS-mutated cancer is selected from the group consisting of: RAS-mutated melanoma, RAS-mutated PDAC, RAS-mutated NSCLC and RAS-mutated CRC, where RAS is any activation mutation in KRAS, NRAS or HRAS.
B-9. The method of any one of embodiments B-1 to B-7, wherein the RAS-mutated cancer is a KRAS G12D or G12V mutation or a NRAS Q61L or Q61K mutation.
B-10. The method of any one of embodiments B-1 to B-7, wherein the RAS-mutated cancer is pancreatic cancer with a KRAS G12C, G12D or G12V mutation.
B-11. The method of any one of embodiments B-1 to B-7, wherein the RAS-mutated cancer is colorectal cancer with a NRAS Q61L or Q61K mutation or colorectal cancer with a KRAS G12D mutation.
B-12. The method of any one of embodiments B-1 to B-7, wherein the RAS-mutated cancer is RAS-mutated melanoma with a NRAS Q61R mutation, or RAS-mutated melanoma with a BRAF V600E mutation.
B-13. The method of any one of embodiments B-1 to B-7, wherein the RAS-mutated cancer is non-small cell lung cancer (NSCLC) with a KRAS G12S mutation.
B-14. The method of any one of embodiments B-1 to B-7, wherein the dual-MEK inhibitor is Compound A administered to the patient once daily in a dose amount of about 240 mg or about 320 mg.
B-15. The method of embodiment B-14, wherein the dual-MEK inhibitor is Compound A administered to the patient once daily in a dose amount of about 240 mg.
B-16. The method of embodiment B-14, wherein the dual-MEK inhibitor is Compound A administered to the patient once daily in a dose amount of about 320 mg.
B-17. The method of any one of embodiments B-14 to B-16, wherein Compound A is administered orally to the patient in need thereof, for at least 28 consecutive days.
B-18. The method of embodiment B-17, wherein the patient is diagnosed with pancreatic cancer with a KRAS G12D mutation.
B-19. The method of embodiment B-17, wherein the patient is diagnosed with colorectal cancer with a KRAS G12V mutation.
B-20. The method of embodiment B-17, wherein the patient is diagnosed with colorectal cancer with a NRAS Q61L mutation.
B-21. The method of embodiment B-17, wherein the patient is diagnosed with colorectal cancer with a NRAS Q61K mutation.
B-22. A method of treating a patient with a cancer with a KRAS G12D or G12V mutation, the method consisting of orally administering to the patient in need thereof a therapeutically effective amount of Compound A,

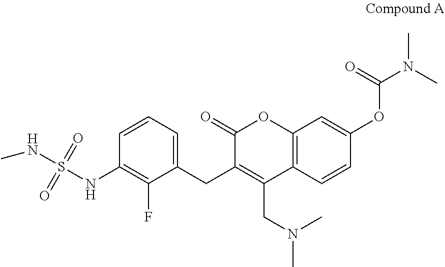

or a pharmaceutically acceptable salt thereof, once daily as a monotherapy.
B-23. The method of embodiment B-22, wherein the patient is diagnosed with pancreatic cancer with a KRAS G12D or G12V mutation.
B-24. The method of embodiment B-22, wherein the patient is diagnosed with colorectal cancer with a KRAS G12D or G12V mutation.
B-25. The method of any one of embodiments B-22 to B-24, wherein the therapeutically effective amount is a dose of 160 mg of Compound A orally once daily as a monotherapy.
B-26. The method of any one of embodiments B-22 to B-24, wherein the therapeutically effective amount is a dose of 240 mg of Compound A orally once daily as a monotherapy.
B-27. The method of any one of embodiments B-22 to B-24, wherein the therapeutically effective amount is a dose of 320 mg of Compound A orally once daily as a monotherapy.
B-28. A method of treating a patient with a cancer with a KRAS G12D or G12V mutation or a cancer with a NRAS Q61L or Q61K mutation, the method consisting of orally administering to the patient in need thereof a total dose of 160 mg of Compound A once daily to treat the cancer,

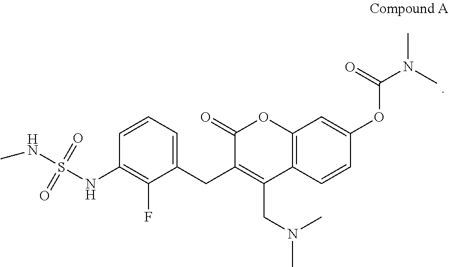

B-29. A method of treating a patient with a cancer with a KRAS G12D or G12V mutation or a cancer with a NRAS Q61L or Q61K mutation, the method consisting of orally administering to the patient in need thereof a total dose of 240 mg of Compound A once daily to treat the cancer,

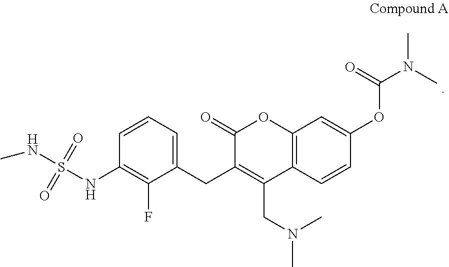

B-30. A method of treating a patient with a cancer with a KRAS G12D or G12V mutation or a cancer with a NRAS Q61L or Q61K mutation, the method consisting of orally administering to the patient in need thereof a total dose of 320 mg of Compound A once daily to treat the cancer,

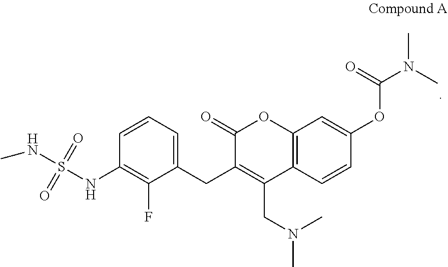

B-31. The method of any one of embodiments B-8 to B-30, wherein Compound A is administered as a monotherapy.
B-32. The method of embodiment B-31, wherein the cancer has a KRAS G12D mutation.
B-33. The method of embodiment B-31, wherein the cancer is pancreatic cancer with a KRAS G12D mutation.
B-34. The method of embodiment B-31, wherein the cancer has a KRAS G12V mutation.
B-35. The method of embodiment B-31, wherein the cancer is colorectal cancer with a KRAS G12V mutation.
B-36. The method of embodiment B-31, wherein the cancer with a NRAS Q61L mutation.
B-37. The method of embodiment B-31, wherein the cancer is colorectal cancer with a NRAS Q61L mutation.
B-38. The method of embodiment B-31, wherein the cancer has a NRAS Q61K mutation.
B-39. The method of embodiment B-31, wherein the cancer is colorectal cancer with a NRAS Q61K mutation.
B-40. A method of treating a patient with a cancer with a mutation in KRAS, NRAS, or HRAS, the method consisting of orally administering to the patient in need thereof a total dose of 160 mg of Compound A once daily to treat the cancer,

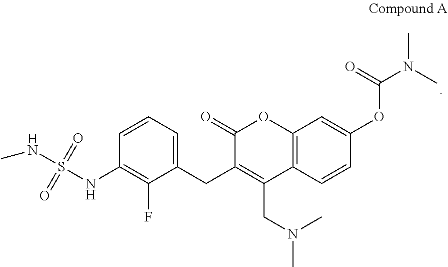

B-41. A method of treating a patient with a cancer with a mutation in KRAS, NRAS, or HRAS, the method consisting of orally administering to the patient in need thereof a total dose of 240 mg of Compound A once daily to treat the cancer,

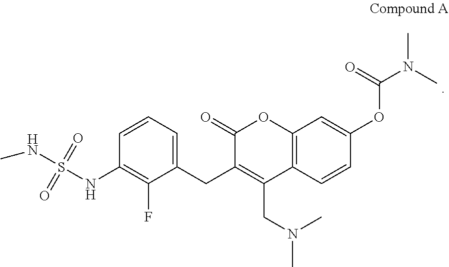

B-42. A method of treating a patient with a cancer with a mutation in KRAS, NRAS, or HRAS, the method consisting of orally administering to the patient in need thereof a total dose of 320 mg of Compound A once daily to treat the cancer,

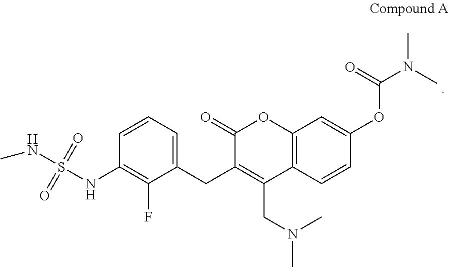

B-43. The method of any one of embodiments B-40 to B-42, wherein the cancer has a KRAS mutation.
B-44. The method of any one of embodiments B-40 to B-42, wherein the cancer has a KRAS G12C, G12D or G12S mutation.
B-45. The method of any one of embodiments B-43 to B-44, wherein the cancer is pancreatic cancer.
B-46. The method of any one of embodiments B-40 to B-42, wherein the cancer has a NRAS mutation.
B-47. The method of any one of embodiments B-40 to B-42, wherein the cancer has a NRAS Q61L or Q61K mutation.
B-48. The method of any one of embodiments B-46 to B-47, wherein the cancer is colorectal cancer.
B-49. The method of any one of embodiments B-40 to B-42, wherein the cancer has a HRAS mutation.
B-50. The method of any one of embodiments B-40 to B-49, wherein Compound A is administered as a monotherapy dual-MEK inhibitor.
B-51. A method of treating a patient with a RAS-mutated cancer, comprising administering once-daily orally a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax at least 2,000 ng/mL and (b) with an interval between consecutive doses of the dual-MEK inhibitor is about seven to eight times a plasma half-life of the dual-MEK inhibitor.
B-52. The method of embodiment B-51, wherein the plasma half-life of the dual-MEK inhibitor in humans is 1-3 hours.
B-53. The method of embodiment B-51, wherein the plasma half-life of the dual-MEK inhibitor in humans is about 2 hours.
B-54. The method of any one of embodiments B-51 to B-53, wherein the dual-MEK inhibitor has a Cmin of over about 0.1 micromolar occurs 6-8 hours after administering the dual-MEK inhibitor free fraction.
B-55. The method of any one of embodiments B-1 to B-6 or B-51 to B-54, wherein the dual-MEK inhibitor is administered for at least 15 consecutive days.
B-56. The method of any one of embodiments B-1 to B-6 or B-51 to B-55, wherein the dual-MEK inhibitor is administered for at least 28 consecutive days.
B-57. A method of treating a patient with a RAS-mutated cancer, the method comprising orally administering to the patient in need thereof a total dose of 240 mg of Compound A once daily to the patient in need thereof, to treat the cancer,

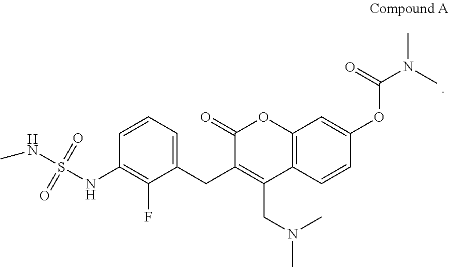

B-58. A method of treating a patient with a RAS-mutated cancer, the method comprising orally administering to the patient in need thereof a total dose of 320 mg of Compound A once daily to the patient in need thereof, to treat the cancer,

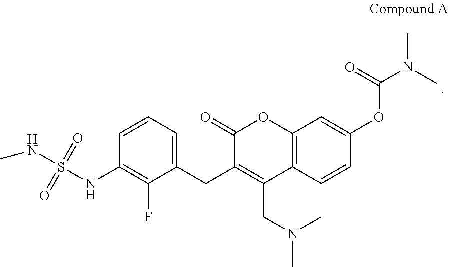

B-59. The method of any one of embodiments B-7 to B-50 or B-57 to B-58, wherein the dual-MEK inhibitor is administered for at least 15 consecutive days.
B-60. The method of any one of embodiments B-7 to B-50 or B-57 to B-58, wherein the dual-MEK inhibitor is administered for at least 28 consecutive days.
B-61. The method of any one of embodiments B-1 to B-7 or B-51 to B-60, wherein the RAS-mutated cancer is any RAS activated mutation in KRAS, NRAS or HRAS.
B-62. The method of embodiments B-61, wherein the RAS-mutated cancer is any RAS activated mutation in KRAS.
B-63. The method of embodiment B-62, wherein the RAS-mutated cancer has a mutation at KRAS G12C.
B-64. The method of embodiment B-62, wherein the RAS-mutated cancer has a mutation at KRAS G12D.
B-65. The method of embodiment B-62, wherein the RAS-mutated cancer has a mutation at KRAS G12V.
B-66. The method of any one of embodiments B-62 to B-65, wherein the cancer is pancreatic cancer.
B-67. The method of embodiment B-61, wherein the RAS-mutated cancer has a mutation at any activation mutation in NRAS.
B-68. The method of embodiment B-67, wherein the RAS-mutated cancer has a mutation at NRAS Q61L.
B-69. The method of embodiment B-67, wherein the RAS-mutated cancer has a mutation at NRAS Q61K.
B-70. The method of embodiment B-67, wherein the RAS-mutated cancer has a mutation at NRAS Q61R.
B-71. The method of any one of embodiments B-64, B-68 or B-69, wherein the cancer is colorectal cancer.
B-72. The method of embodiment B-70, wherein the cancer is melanoma.
B-73. The method of embodiment B-62, wherein the RAS-mutated cancer has a mutation at KRAS G12S.
B-74. The method of embodiment B-73, wherein the cancer is NSCLC.
B-75. The method of embodiment B-61, wherein the RAS-mutated cancer is any activation mutation in HRAS.
B-76. A method of treating cancer, the method comprising administering orally once-daily a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax is at least 2,000 ng/mL and (b) with an interval between consecutive doses of the dual-MEK inhibitor is about seven to eight times a plasma half-life of the dual-MEK inhibitor.
B-77. The method of embodiment B-76, wherein the plasma half-life of the dual-MEK inhibitor in humans is 1-3 hours.
B-78. The method of embodiment B-77, wherein the plasma half-life of the dual-MEK inhibitor in humans is about 2 hours.
B-79. The method of any one of embodiments B-76 to B-78, wherein the dual-MEK inhibitor has a Cmin of over about 0.1 micromolar occurs 6-8 hours after administering the dual-MEK inhibitor free fraction.
B-80. The method of any one of embodiments B-76 to B-79, wherein the dual-MEK inhibitor is administered for at least 15 consecutive days.
B-81. The method of any one of embodiments B-76 to B-80, wherein the dual-MEK inhibitor is administered for at least 28 consecutive days.
B-82. A method of treating cancer in a patient, the method comprising orally administering to the patient in need thereof a total dose of 240 mg of Compound A once daily to the patient in need thereof,

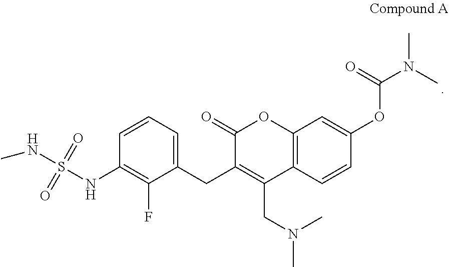

B-83. A method of treating cancer in a patient, the method comprising orally administering to the patient in need thereof a total dose of 320 mg of Compound A once daily to the patient in need thereof,

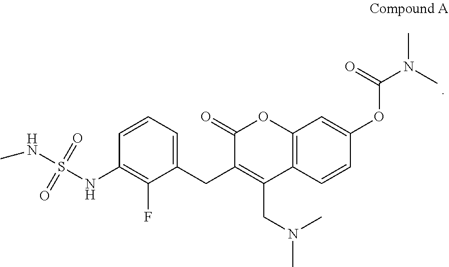

B-84. The method of any one of embodiments B-76 to B-83, wherein the cancer has a MAPK mutation selected from the group consisting of: KRAS-G12S, BRAF-V600E, NRAS-Q61R, KRAS-G12C, NRAS-Q61K, or KRAS-G12D.
B-85. The method of embodiment B-84, wherein the cancer is human lung adenocarcinoma.
B-86. The method of embodiment B-84, wherein the cancer is human malignant melanoma.
B-87. The method of embodiment B-84, wherein the cancer is human NSCLC.
B-88. The method of embodiment B-84, wherein the cancer is human colon carcinoma.
B-89. The method of any one of embodiments B-76 to B-83, wherein the cancer has a KRAS, BRAF, NRAS, or MAPK mutation.
B-90. The method of embodiment B-89, wherein the cancer has a KRAS-G12S mutation.
B-91. The method of embodiment B-89, wherein the cancer has a BRAF-V600E mutation.
B-92. The method of embodiment B-89, wherein the cancer has a NRAS-Q61R mutation.
B-93. The method of embodiment B-89, wherein the cancer has a KRAS-G12C mutation.
B-94. The method of embodiment B-89, wherein the cancer has a NRAS-Q61K mutation.
B-95. The method of embodiment B-89, wherein the cancer has a KRAS-G12D mutation.
B-96. The method of embodiment B-17, wherein the patient is diagnosed with pancreatic cancer with a KRAS G12V mutation.
B-97. The method of embodiment B-17, wherein the patient is diagnosed with colorectal cancer with a KRAS G12S mutation.

### EXAMPLES

Additional embodiments are disclosed in further detail in the following representative examples, which are not in any way intended to limit the scope of the claims. The following examples are provided for the guidance of the reader, and represent certain methods for making and using the compounds and compositions exemplified herein. These methods are not limiting, and it will be apparent that other routes may be employed to prepare these compounds. The compound numberings used in the synthetic schemes depicted below are meant for those specific schemes only, and should not be construed as or confused with same numberings in other sections of the application.

### Example 1

### Synthesis of Compound A (or Compound 9)

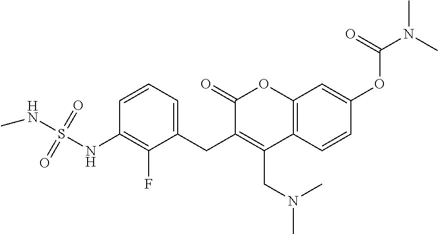

Compound A was prepared in 1 step:

4-(bromomethyl)-3-(2-fluoro-3-((N-methylsulfamoyl)amino)benzyl)-2-oxo-2H-chromen-7-yl dimethylcarbamate (22.22 g, 34.79 mmol) was suspended in methanol. Dimethylamine 2M was added and the formed reaction mixture was stirred until full conversion was observed. After full conversion the reaction was concentrated under reduced pressure. 1M HCl was added to the residue and the water layer was extracted with CH₂Cl₂. The water layer was made basic with solid Na₂CO₃. The basic water layer was extracted with CH₂Cl₂. The organic layer from the basic extraction was washed with brine, dried over Na₂SO₄, filtered and concentrated under reduced pressure to obtain the title compound (13.23 g, 25.7 mmol, yield: 74%) as a light yellow solid.

Yield: Compound A was isolated as a light yellow solid (74% over 1 step). Analysis: LCMS (Method T): t_{R}=1.53 min; m/z calculated for [M-H]⁺=507.2, found=507.2; ¹H NMR (400 MHz, DMSO) δ 9.38 (s, 1H), 8.08 (d, J=8.8 Hz, 1H), 7.28 (td, J=8.0, 1.6 Hz, 1H), 7.25-7.18 (m, 2H), 7.15 (dd, J=8.8, 2.4 Hz, 1H), 7.00 (t, J=7.9 Hz, 1H), 6.90-6.77 (m, 1H), 4.04 (s, 2H), 3.64 (s, 2H), 3.06 (s, 3H), 2.93 (s, 3H), 2.52 (d, J=4.9 Hz, 3H), 2.19 (s, 6H).

### Example 2

### Compound A Nonclinical Pharmacology

Compound A is a potent, non-adenosine 5′-Triphosphate (ATP) competitive selective dual MEK inhibitor with the biochemical inhibitory half-maximal inhibitory concentration (IC₅₀) for activated ERK of 24.2 nM to 83.2 nM across several cancer disease types. Compound A has been shown to have in vivo anti-tumor activity in mouse syngeneic and human xenograft models of activating MAPK pathway mutations in KRAS, NRAS, HRAS, BRAF, neurofibromatosis type 1 (NF1), and KSR, with the most sensitive being predominantly melanoma and pancreatic cancer cell lines that harbor activating MAPK pathway mutations in KRAS, NRAS, HRAS, BRAF, NF1, and KSR (Table 3).

TABLE 3
(IC₅₀ values for pERK in Multiple 2D Grown Cell Lines)

| Tumor cell line and MAPK | Key MAPK Activating | IC₅₀ |
|---|---|---|
| mutation | Mutation(s) | nM^{a} |
| A549 KRAS^{G12S} | KRAS:p.G12S, | 24.16 to |
| Human lung | KSR1:Amplified, | 36.63 |
| adenocarcinoma | SOS1:Amplified | |
| A375 BRAF^{V600E} | BRAF:p.V600E:class1, | 37.65 to |
| Human malignant melanoma | MAP2K2:Amplified | 83.19 |
| SK-MEL-2 NRAS^{Q61R} | NRAS:p.Q61R, | 26.19 to |
| Human pancreatic | BRAF:Amplified, | 36.17 |
| adenocarcinoma | RAF1:Amplifeid, | |
| | MAP2K2:Amplified | |
| MIA PaCa-2 KRAS^{G12C} | KRAS:p.G12C, | 49.39 |
| Human pancreatic | NRAS:Amplified, | |
| adenocarcinoma | BRAF:Amplified, | |
| | MAP2K2:Amplified, | |
| | KSR1:Amplified | |
| NCI-H1299 NRAS^{Q61K} | KRAS:Amplified, | 66.46 |
| Human non-small | NRAS:p.Q61K, | |
| cell lung cancer | BRAF:Amplified, | |
| | MAP2K2:Amplified | |
| SK-MEL-28 BRAF^{V600E} | KRAS:Amplified, | 29.86 |
| Human malignant melanoma | BRAF:p.V600E:class1; | |
| | Amplified, | |
| | FAF1:Amplified, | |
| | MAPK1:Amplified, | |
| | SOS1:Amplified | |
| CT26 KRAS^{G12D} | KRAS:p.G12D (mouse | 46.26 |
| Murine colon carcinoma | cell line; known | |
| | mutation) | |

2D = two dimensional;
Amp = amplified;
BRAF = B isomer of rapidly accelerated fibrosarcoma (RAF);
IC50 = 50% inhibitory concentration;
KRAS = K isomer of rat sarcoma (RAS);
KSR = kinase suppressor of Ras;
MAP2K2 = mitogen-activated protein kinase kinase 2;
MEL = melanoma;
NF1 = neurofibromatosis type 1;
NRAS = N isomer of rat sarcoma (RAS);
RAF1 = Raf-1 proto-oncogene, serine/threonine kinase;
SOS1 = son of sevenless 1
^{a}Potency observed in cell-based assays (range shown when experiment was repeated)

Corresponding reductions in phosphorylated MEK (pMEK) were observed within 2-hours following treatment of cells with 100 nM Compound A, confirming that Compound A was not sensitive to the MAPK pathway feedback reactivation events, such as CRAF-bypass, that are a known limitation of first generation MEK inhibitors currently in clinical use.

### Example 3

### Preclinical Studies: Pharmacogenomics Data

In a pharmacogenomics study utilizing a colorectal KRAS G12D tumor model in BALB/c mice, downstream ERK inhibition of the MAPK pathway was evaluated after Compound A treatment.

To demonstrate the ability of Compound A to achieve deep, cyclic inhibition, the downstream transcriptomic activity following Compound A exposure (100 mg/kg BID PO) was evaluated and compared to selumetinib (100 mg/kg BID PO) in the Colon-26 model. RNA sequencing of tumors allowed for interrogation of the 20 most common genes associated with ERK-induced transcription. Analysis was performed at 2 h and 12 h post-dose after 18 days of chronic treatment administration.

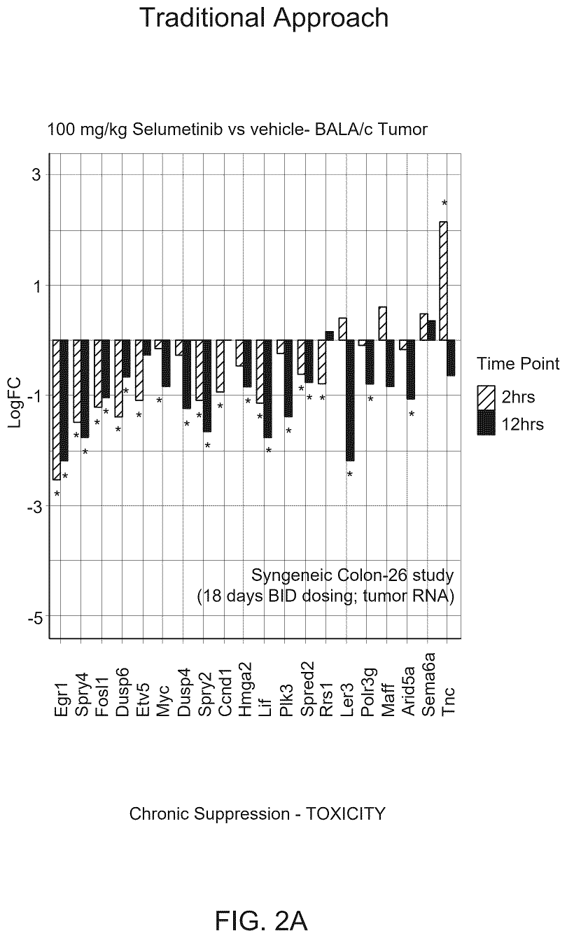

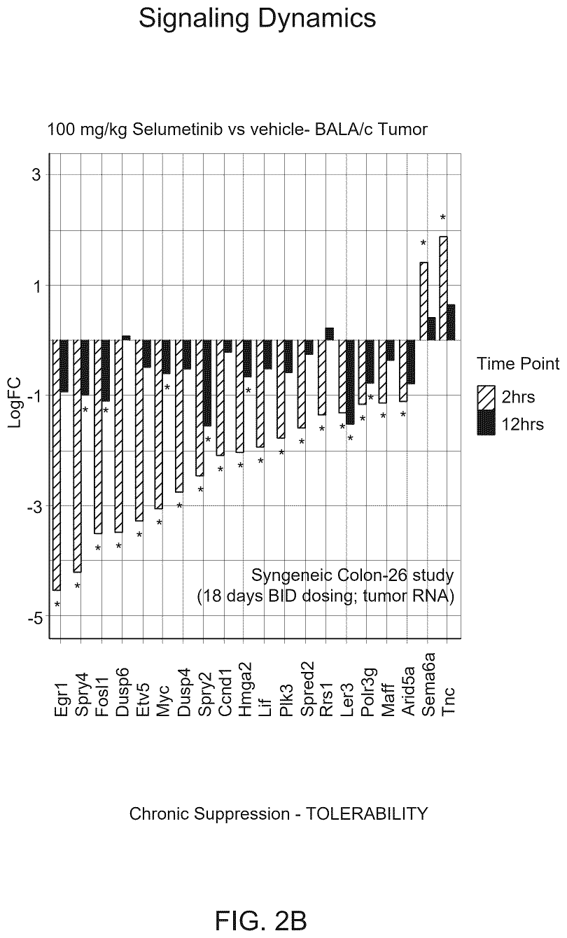

The vehicle, selumetinib and Compound A was orally administered twice a day at 100 mg/kg/dose, then harvested the tumors after 18 days of chronic treatment at 2 and 12 hours following the last drug dose to evaluate RNAseq changes. The tumors were collected across distinct BALB/c mice and RNAseq changes were evaluated using statistical analysis software. Consistent with Compound A's designed short plasma half-life, deep, cyclic inhibition of most of the top genes in the ERK transcriptome was observed, as noted by the differences of the dark and light bars, which suggests the potential for improved tolerability by allowing healthy normal cells to regenerate before the next dose is administered. For example, Erg1 and Spry4 were both downregulated over 16-fold at 2 hours after receiving the first dose on day 18 of the study, and at 12 hours after the first dose, which was prior to the second dose, both genes were approaching their baseline state when compared to vehicle treated tumors (FIG. 2). In contrast to Compound A, deep cyclic inhibition by selumetinib was not observed, but rather sustained MAPK pathway suppression versus vehicle groups between the two timepoints on day 18 was observed (FIG. 2). The top 20 genes were a subset of a 52-gene signature for ERK signaling.

The data showed that, compared with selumetinib, Compound A resulted in a greater difference in transcriptomic activity at Cmax and at trough in most of the genes evaluated.

FIG. 2 is a graph showing data from a head-to-head comparison of Compound A against selumetinib using a Colon-26 syngeneic tumor model. The data shows deep cyclic inhibition of the ERK transcriptome.

### Example 4

### Preclinical Studies: Compound A in a KRAS Mutant NSCLC Tumor Model

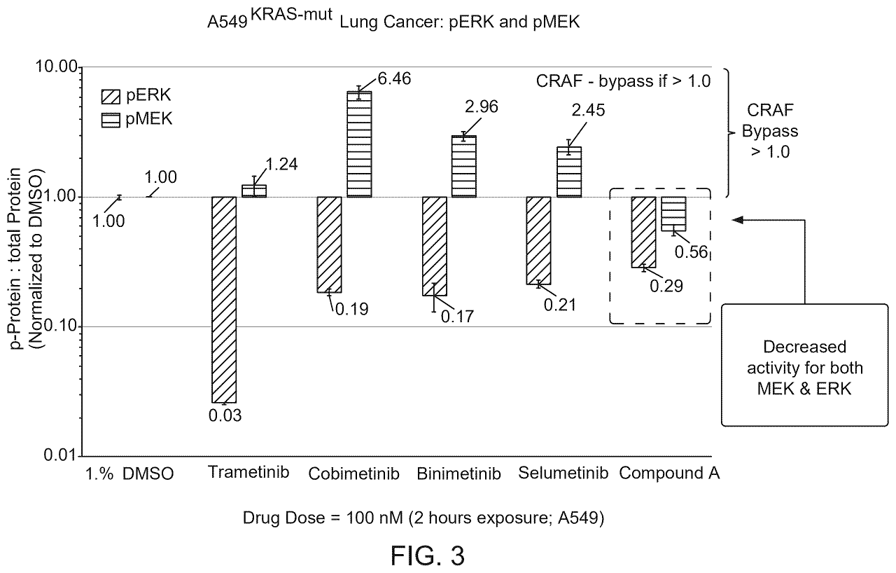

Compound A was evaluated head-to-head against four FDA-approved MEK inhibitors for CRAF-bypass resistance in a KRAS mutant NSCLC tumor model. The tumor cells with 100 nM of each drug were exposed for 2 hours and MEK and ERK activation levels were evaluated. Compound A was observed to reduce overall activity of the MAPK pathway at ERK and pathway reactivation at MEK through a decrease in MEK and ERK activation, resulting in CRAF-bypass resistance. In contrast, it was observed that all four FDA-approved MEK inhibitors displayed an increase in activated MEK, resulting in CRAF-bypass (FIG. 3).

As depicted in FIG. 3, while each MEK inhibitor effectively inhibited ERK, as evidenced by decreases in pERK (vertical slash bars), the FDA-approved MEK inhibitor all demonstrated an increase in cellular pools of pMEK (red bars), indicative of a reactivation event through CRAF bypass. Only Compound A inhibited both pERK and pMEK in the RAS-mutated setting. Compound A acts as a dual MEK inhibitor.

FIG. 3 shows results from a head-to-head comparison of Compound A against four FDA-Approved MEK Inhibitors Using a A549 Xenograft Tumor Model, showing Compound A prevented downstream activation of ERK (↓ pERK) and inhibited activation of MEK (↓ pMEK).

### Example 5

### Preclinical Studies: Compound A in a Colon-26 Tumor Model

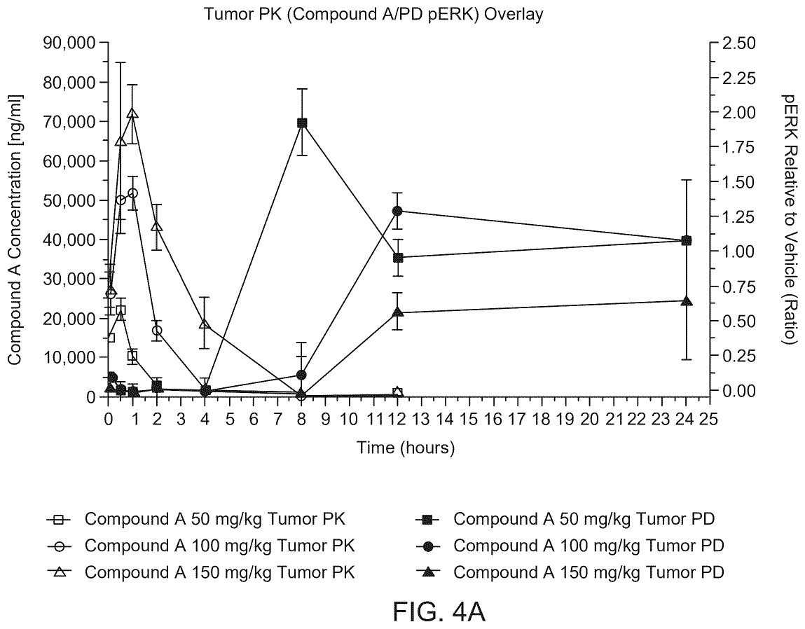

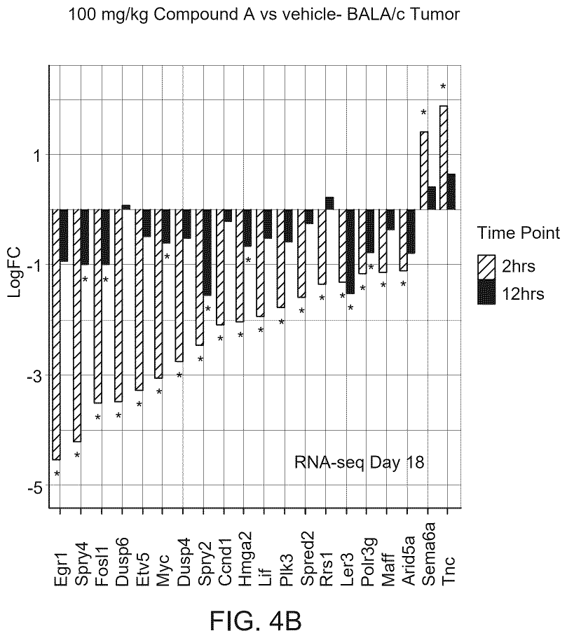

In the Colon-26 model, Compound A transiently inhibited activated pERK in a dose-dependent manner (FIG. 4A) at doses ranging from 12.5 to 150 mg/kg PO after a single dose (12.5 and 25 mg/kg not shown). This suppression correlated with plasma and tumor exposures, showing that when levels dropped below 50% pERK inhibition (IC₅₀) levels compared to vehicle from in vitro 2D assays (24.16 nM to 83.90 nM) the attenuation was reversed. Further evidence supporting the selective deep cyclic inhibition was shown at the genomic level, where Compound A attenuated the expression of several ERK signaling pathway genes when dosed at 100 mg/kg BID PO for 18 days. As an example, on day 18 of the study Egr1 and Spry4 were both downregulated by over 16-fold, 2 hours after receiving the first dose (note: log 2FC scale), while at 12 hours after the first dose, representing the drug trough in a BID schedule, both genes were approaching their baseline state when compared to vehicle treated tumors (FIG. 4B). In FIG. 4B, Arid5a=AT Rich Interactive Domain 5A; Ccnd1=cyclin D1 protein; Dusp6=dual specificity phosphatase 6; Egr1=early growth response protein 1; EVH1=Ena/Vasp Homology domain 1; Hmga2=high mobility group AT-hock 2; Ier3=immediate early response 3; Lif=leukemia inhibitory factor; Maff=musculoaponeurotic fibrosarcoma oncogene homolog F; PD=pharmacodynamics; PK=pharmacokinetics; pERK=phosphorylated extracellular signal-regulated kinase (or mitogen-activated protein kinase); PIk3=phosphoinositide 3-kinase; Polr3g=RNA Polymerase III Subunit G; Rrsl=Ribosome Biogenesis Regulator 1 Homolog; RTK=receptor tyrosine kinase; Sema6a=Semaphorin 6A; Spred2=sprouty-related EVH1 domain-containing protein 2; Spry2=sprouty RTK signaling antagonist 2; Spry4=sprouty RTK signaling antagonist 4; Tnc=tenascin C.

Referring to FIG. 4A and FIG. 4B: Syngeneic Colon-26 (KRASG12D mutant) colorectal tumor bearing mice treated with Compound A. Composite graph (FIG. 4A) shows tumor PK and PD (pERK) quantified at the times indicated following a single PO dose. Top genes in the ERK transcriptome from tumor treated with 100 mg/kg BID PO Compound A (FIG. 4B) were expressed as log fold change (* adj p-value<0.05 vs. vehicle control). Tumors were taken at 2-hour (dark bars) or 12-hour (light bars) as last dose on day 18.

Compound A was evaluated head-to-head against binimetinib and selumetinib in an aggressive murine colorectal tumor model (i.e., Colon-26), which expresses mutant KRAS-G12D. Both binimetinib and selumetinib have relatively short half-lives (3.5 hr and 6.2 hr, respectively).

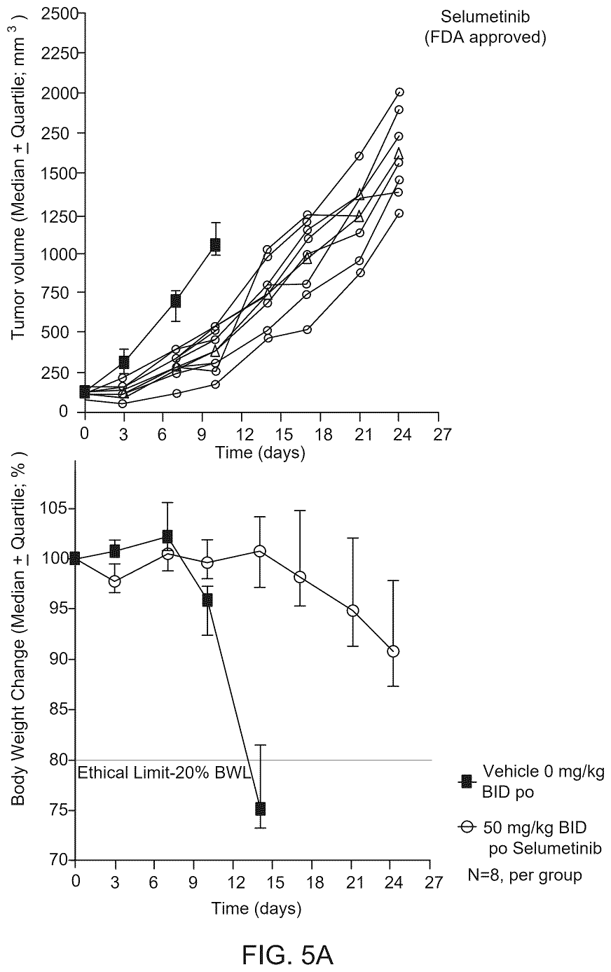

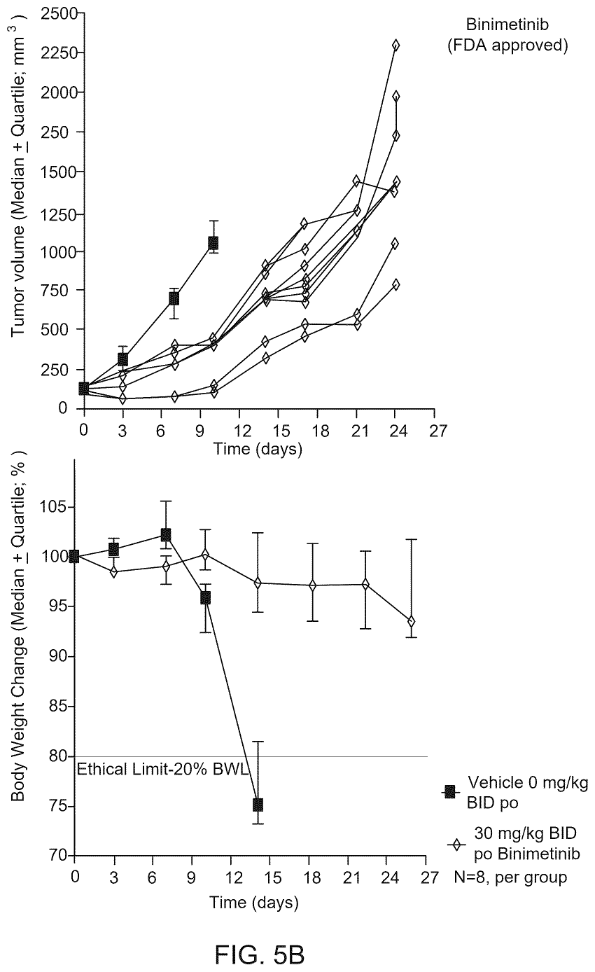

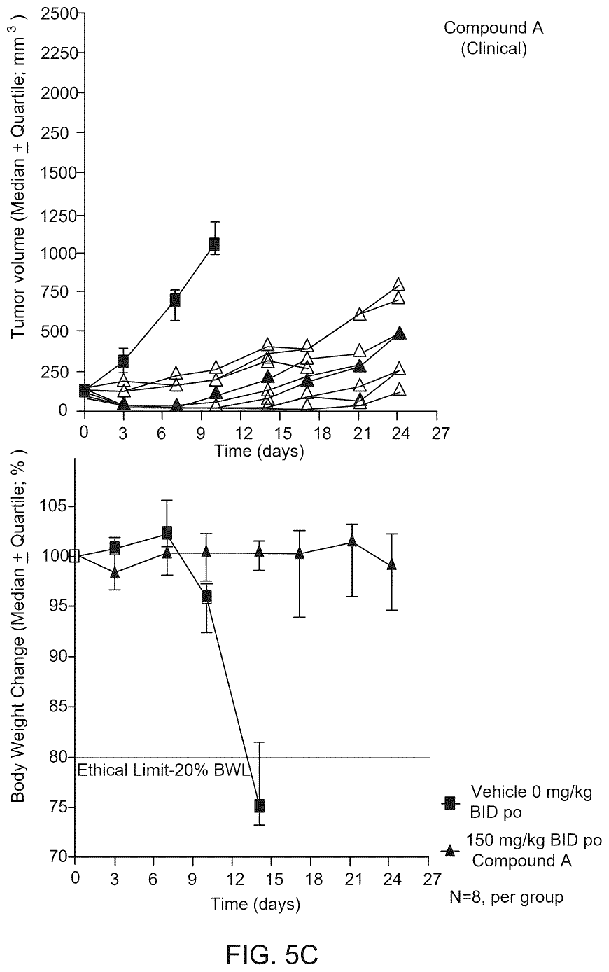

Compound A was observed to demonstrate greater tumor growth inhibition, where notably 5 of 8 mice experienced tumor regression during the first 10 days of dosing, as well as greater tolerability, evidenced by changes in BWL. In addition, Compound A was observed to have overall better durability of antitumor response as compared to the two FDA-approved MEK inhibitors, as demonstrated by significantly lower tumor volume, or TV, progression. This study demonstrated that Compound A as compared to binimetinib and selumetinib provided greater tumor inhibition, lower BWL and lower TV progression (as depicted in FIGS. 5A-C).

FIGS. 5A-C are graphs showing the head-to-head comparison of Compound A against binimetinib and selumetinib using a Colon-26 syngeneic tumor model (body weight loss and tumor volume observed). Colon-26 (KRAS-G12D) syngeneic colorectal tumor model in balb/c mice. Selumetinib and binimetinib were commercially purchased. Tumor Growth Inhibition; TGI %=[1−(Ti−T₀)/(Ci−C₀)]×100; T=treatment groups; C=control groups; expanded TGI formula vs. previous 1−[T/C)]×100% method.

### Example 6

### Preclinical Studies: Compound A in A549 NSCLC Xenograft Tumor Model

After observing the results of the Colon-26 tumor study, two follow-up in vivo studies were completed, where Compound A was evaluated head-to-head against binimetinib or encorafenib, a BRAF inhibitor, as monotherapy plus the combination of binimetinib with encorafenib in BALB/c mice tumor models with RAS and RAF mutations. It should be noted that when encorafenib is used to treat KRAS mutant tumors that are wild type for BRAF, it can paradoxically activate the MAPK pathway and antagonize the effects of binimetinib. In addition, the drug doses and schedules used for binimetinib and encorafenib in these studies were consistent with or exceeded what was provided in their NDAs to the FDA for human-relevant exposures in rodent models.

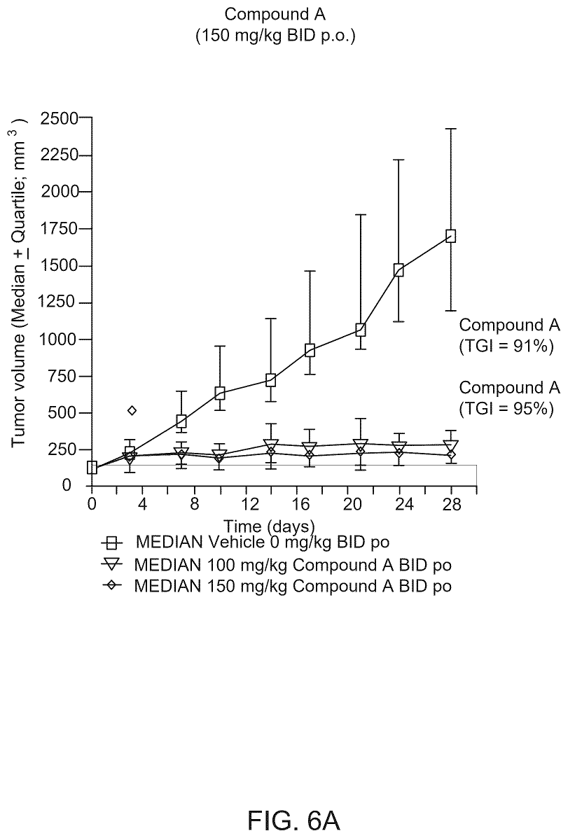

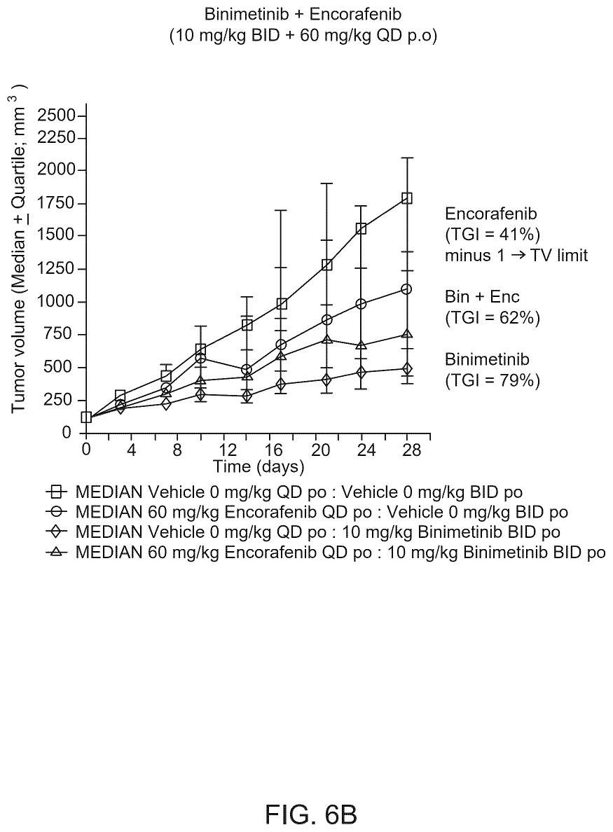

Compound A was evaluated head-to-head against binimetinib monotherapy and in combination with encorafenib in the KRAS G12S human NSCLC xenograft tumor model (i.e., A549). When comparing Compound A to binimetinib monotherapy, Compound A was observed to have greater tumor growth inhibition (FIG. 6A). The observations of Compound A head-to-head against binimetinib alone and in combination with encorafenib, which was not considered relevant for a KRAS mutant, RAF wild-type tumor model (FIG. 6B).

FIGS. 6A and 6B are graphs showing data from head-to-head comparison of Compound A against binimetinib+/−encorafenib using an A549 xenograft tumor model (tumor volume). A549 (KRAS^{G12S}) NSCLC Xenograft Tumor Model in Athymic Nude Mice. Binimetinib and encorafenib were commercially purchased. Tumor Growth Inhibition; (TGI) %=[1−(Ti−T₀)/(Ci−C₀)]×100; T=treatment groups; C=control groups; expanded TGI formula vs. previously 1−[T/C]×100% method. Human Dose Equivalent (HDE) binimetinib=1-3 mg/kg BID.

### Example 7

### Preclinical Studies: Compound A in A375 (BRAF V600E) Melanoma Xenograft Tumor Model

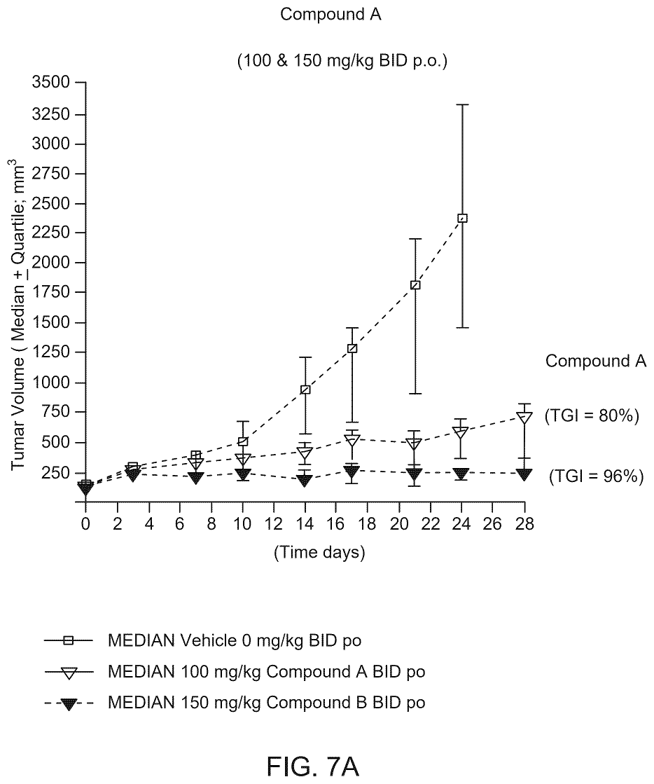

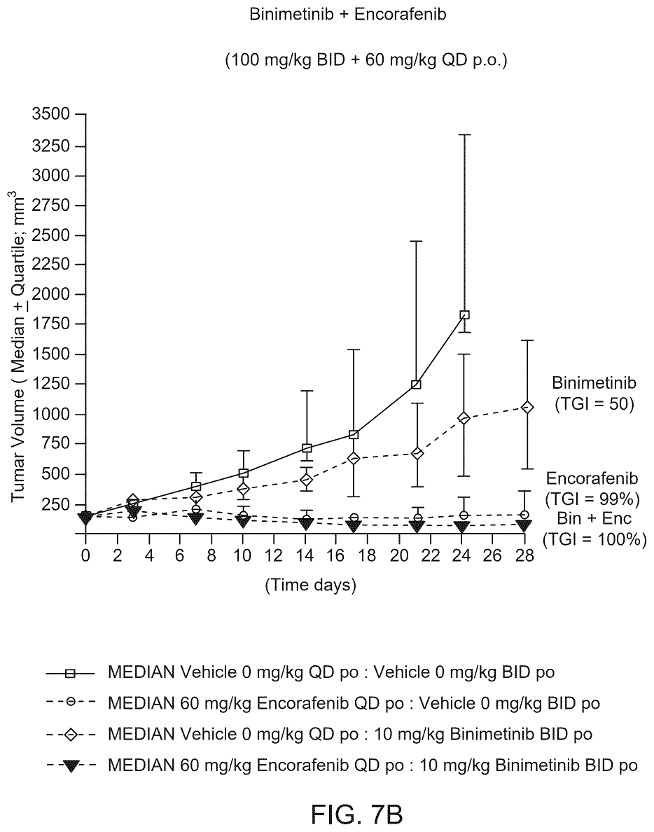

Compound A was evaluated head-to-head against binimetinib and encorafenib monotherapy and the combination of binimetinib with encorafenib in a BRAF V600E human melanoma xenograft model. It should be noted that the administered combination of binimetinib and encorafenib for BRAF mutant melanoma, such as BRAF V600E/K, is an FDA-approved combination. When comparing Compound A alone to binimetinib in combination with encorafenib, it was observed that the combination therapy had greater tumor growth inhibition (FIG. 7A). However, Compound A was compared to binimetinib monotherapy, it was observed that Compound A had greater tumor growth inhibition (FIG. 7B).

FIGS. 7A and 7B are graphs showing data from the head-to-head comparison of Compound A against binimetinib+/−encorafenib using a A375 xenograft tumor model (tumor volume). A375 (BRAFV600E) Melanoma Xenograft Tumor Model in Athymic Nude Mice. Binimetinib and encorafenib were commercially purchased. Tumor Growth Inhibition; (TGI) %=[1−(Ti−T₀)/(Ci−C₀)]×100; T=treatment groups; C=control groups; expanded TGI formula vs. previously 1−[T/C]×100% method. Human Dose Equivalent (HDE) binimetinib=1-3 mg/kg BID.

### Example 8

### Preclinical Studies: Compound A in MIA PaCa-2 (KRAS G12C) and Capan-2 (KRAS-G12V)

### Pancreatic Cancer Xenograft Tumor Models

Compound A was evaluated head-to-head against sotorasib (AMG-510) and gemcitabine alone, and Compound A in combination with sotorasib, for 21 days in the KRAS G12C xenograft model (i.e., MIA PaCa-2 (KRAS) Pancreatic Xenograft Tumor Model in Athymic Nude Mice). Compound A as compared to sotorasib demonstrated tumor regression, both with insignificant BWL. Comparing Compound A alone, against sotorasib and in combination with sotorasib, tumor regressions with insignificant median BWL were observed (i.e., within 3% of baseline), which indicates activity, durability and tolerability of Compound A against a KRAS G12C mutant pancreatic cancer model. FIG. 8A shows data from the head-to-head Comparison of Compound A+/−sotorasib (AMG-510) using a MIA PaCa-2 Xenograft Tumor Model: Tumor Volume

Compound A was evaluated head-to-head against sotorasib (AMG-510) and adagrasib alone for 21 days in the Capan-2 (KRAS G12V) Pancreatic Xenograft Tumor Model in Athymic Nude Mice. As shown in FIG. 8B, Compound A demonstrated tumor regression as compared to sotorasib or adagrasib, with insignificant BWL.

In FIG. 8A and FIG. 8B: Tumor Growth Inhibition (TGI) %=[1−(Ti−T0)/(Ci−C0)]×100%; Expanded TGI formula vs. previous 1−[T/C]×100% method.

### Example 9

### Compound A in SK-MEL-2 (NRAS Q61R) Melanoma Xenograft Tumor Model

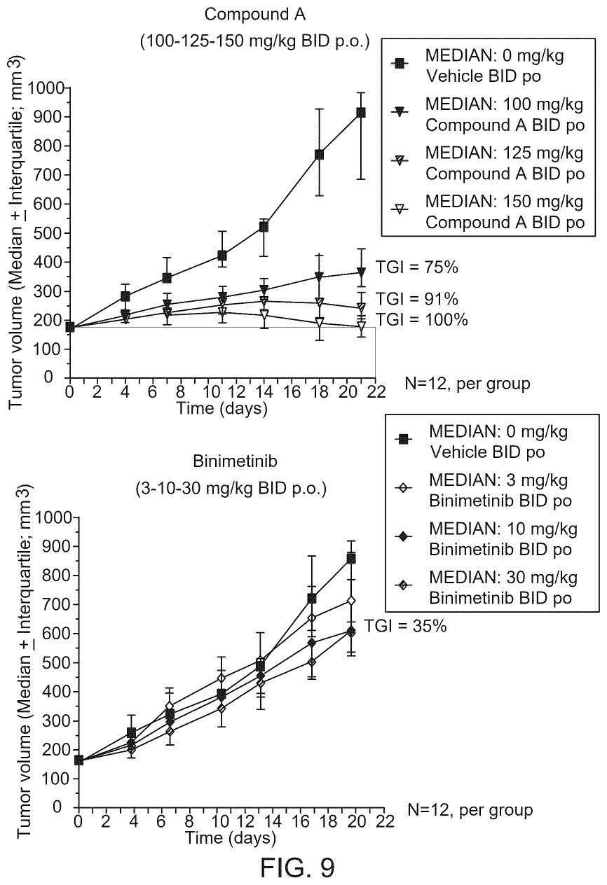

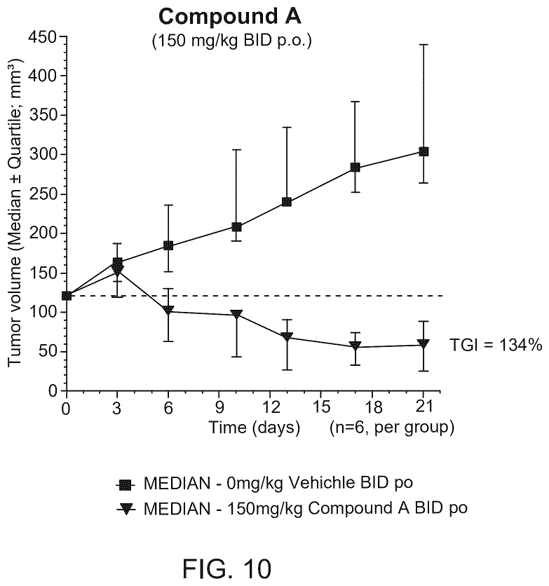

Compound A was evaluated as a monotherapy as compared to binimetinib for 21 days in the NRASQ61R mutant tumor model (i.e., SK-MEL-2). Superior tumor growth inhibition was observed relative to binimetinib. FIG. 9 shows results from a head-to-head comparison of tumor volume measured with Compound A against binimetinib in a NRAS-Q61R SK-MEL-2 melanoma xenograft study. Compound A as compared to binimetinib monotherapy demonstrated greater tumor growth inhibition (TGI), as shown in FIG. 9. Midcycle tumor regressions in mice treated with Compound A was observed, which demonstrated activity and durability of Compound A against an NRAS Q61R mutant melanoma cancer model (FIG. 10).

FIG. 10 is a graph showing data obtained from an evaluation of Compound A as compared to vehicle using a SK-MEL-2 xenograft tumor model (Tumor Volume). SK-MEL-2 (NRAS^{Q61R}) Melanoma Xenograft Tumor Model in Athymic Nude Mice. Tumor Growth Inhibition; (TGI) %=[1−(Ti−T0)/(Ci−C₀)]×100; T=treatment groups; C=control groups; expanded TGI formula vs. previously 1−[T/C]×100% method.

A Capan-2 PDAC xenograft animal model was utilized to evaluate single agent activity of Compound A (100, 150 mg/kg BID p.o.) vs. sotorasib and adagrasib (30 and 100 mg/kg QD p.o. each) for 21 days treatment after tumors had reached volumes of 150 to 200 mm³. A head-to-head comparison in vivo demonstrated no Tumor Growth Inhibition (TGI) by sotorasib or adagrasib in KRAS-G12V mutant Capan-2 PDAC tumors, while Compound A prompted TGIs up to 84% at 150 mg/kg BID po.

### Example 10

### Humanized 3D Tumor Models

Novel dual-MEK inhibitor Compound A is under clinical investigation for use in RAS-addicted solid tumors. Approved KRAS G12C inhibitors are available but cover a limited subset of patients. For example, the KRAS G12C substitution occurs in only 1-3% of pancreatic cancers. The response to Compound A was assessed across RAS mutant preclinical models to determine whether a preference was observed for mutation position, or for specific amino acid substitutions.

Response to Compound A was measured in a humanized 3D tumor growth assay across 132 tumor models. Seventy-five of these models have a reported RAS mutation, and all models are being mutationally profiled by whole exome sequencing, with the majority (~85%) completed to date. The RAS-mutant panel spans 12 tissue types and includes a subset of 30 confirmed KRAS G12 mutated cell lines drawn from three major indications: 12 pancreatic, 11 lung, and 7 colorectal cancer models. Based on the 3D assay, cell lines were classified into sensitive, intermediate, and resistant to Compound A. The distribution of responses was then assessed across mutation position and amino acid substitutions.

Association of response with amino acid identity was further evaluated in a subset of lines for the most frequently altered residue in KRAS, G12. A distribution of responses was observed for each amino acid substitution. The G12 substitutions having at least four cell lines each total across the three indications included G12C (8 lines), D (5 lines), R (4 lines), and V (11 lines). Across cell lines for each of these substitutions, multiple response categories were observed. In each case, half or more lines fell into the intermediate category with the rest falling into one or both of the other response categories. For example, out of the 8 KRAS G12C lines, 6 showed intermediate response, 1 showed resistance, and 1 showed sensitivity. Examining these distributions together, no significant statistical relationship was seen between the amino acid substitution and response categories by Fisher's exact test.

Across 75 RAS-mutated cell lines, each mutation position or amino acid substitution was associated with sensitive or intermediate response in at least one line. Looking at the most commonly mutated position in KRAS, across 28 KRAS G12 mutated cell lines from three cancer indications, no significant preference was observed with respect to response to Compound A for a particular amino acid at G12. These observations suggest potential relevance of Compound A to a broad RAS-driven patient population.

Compound A, with Universal-RAS activity through Deep Cyclic Inhibition (DCI) of MEK, was evaluated in humanized 3D preclinical tumor models displaying diverse MAPK pathway activation events. Based on drug sensitivity and resistance profiles, a biomarker signature for Compound A was developed in order to project potential therapeutic response of cancer patients found in the AACR Project GENIE database.

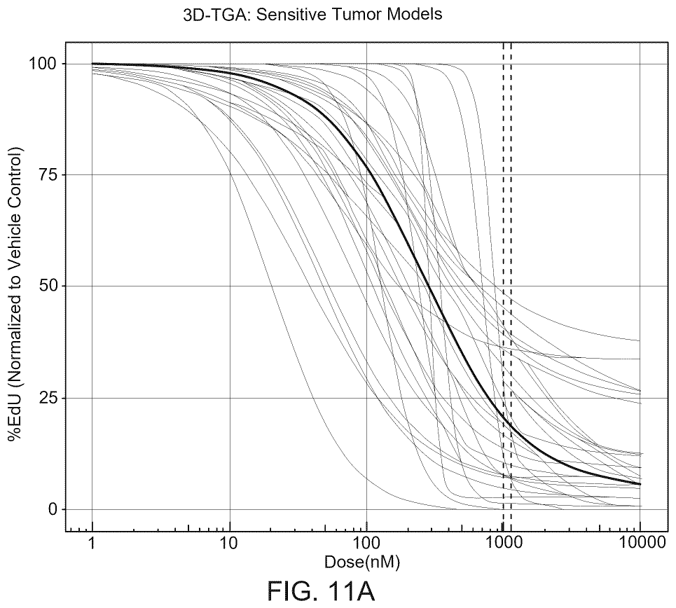

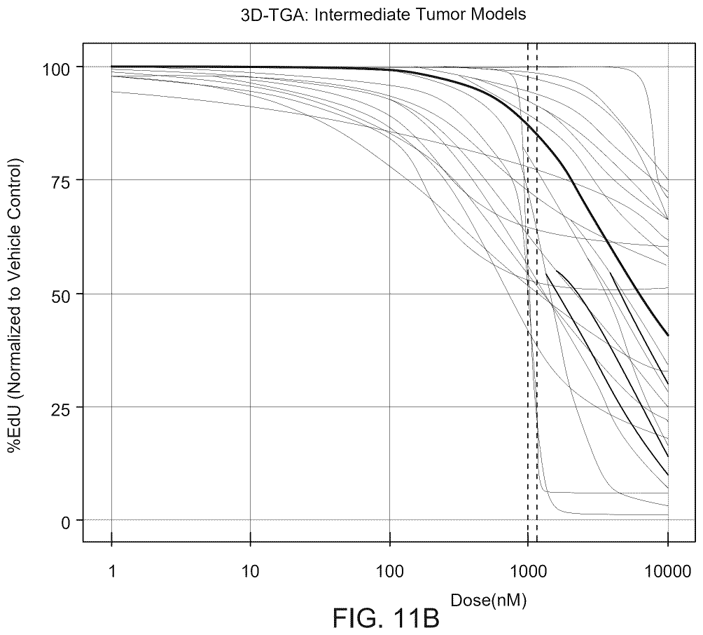

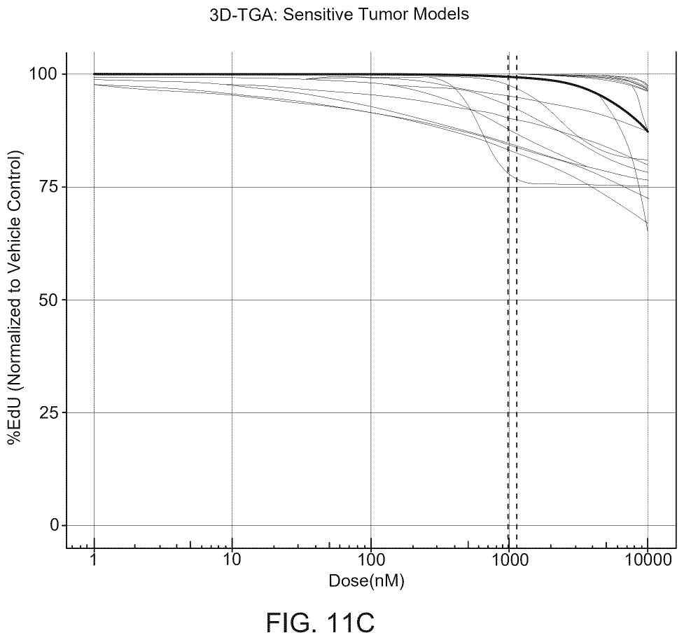

Humanized 3D preclinical models better predict in vivo tumor responses versus 2D culture and more accurately replicate biology of human tumors. Therefore, the antitumor activity of Compound A was evaluated in over 132 tumor models spanning 12 distinct tumor types in the humanized 3D tumor growth assay (3D-TGA) (see, FIGS. 11A-11C). Cell-based whole exome sequencing (WES) readouts were combined with 3D-TGA results to build a pharmacogenomic response algorithm. When applied to the GENIE patient database, resultant tumor-specific response landscapes helped to inform an early Universal-RAS clinical trial design for Compound A (see, FIGS. 11A-11C).

Cell lines tested in 3D-TGA (N=132) were assigned response of sensitive (IC50<1 uM), intermediate (IC50≥1 and >25% reduction at 10 uM), and resistant otherwise The dark line on each plot represents the median of the individual curves; Dotted vertical lines match Cmax Compound A drug free-fractions attained in patients 3 and 4 at 160 mg QD p.o.

For 122 of the 3D-TGA cell lines where in-house WES data were also collected, the upper plateau-to-1 μM difference was used to delineate responder from non-responder, and thus train a logistic regression model with 10-fold cross validation. There were 28 features used, one comprised of a consolidated MAPK mutations status (mutations in K/H/NRAS, GNAQ/11, and BRAF class I/II), and 27 other genes representing key pathways. With an AUC of 0.84 on the training cohort and 0.72 on test cohort, the model was then applied to GENIE cohorts (see, FIGS. 11A-11C) where all features were included on the testing panel. For each indication of interest, a kernel density plot of the probability of predicted response for the entire cohort (projected onto the negative y-axis), and the MAPK(+) subset (positive y-axis) shows enrichment for higher probability of response with restriction to MAPK alteration status. FIG. 11A, FIG. 11B and FIG. 11C show 3D-TGA Compound A Dose Responses (N=132 Models; 12 Tumor Indications).

The depth of response to Compound A was evaluated across a panel of diverse 3D-TGA tumor models and led to identification of a biomarker signature for therapeutically addressable MAPK pathway addiction. To translate these findings into a relevant clinical application, a response algorithm was developed and applied to the GENIE database, which has cataloged the molecular profiles of over 100,000 cancer patients. Mutational landscapes of patients within GENIE helped identify preclinical models that better represent patient profiles likely to be encountered in the clinic. This approach could, as a general principle, be applied as a tool for improving biomarker discovery and clinical translation of oncology drugs.

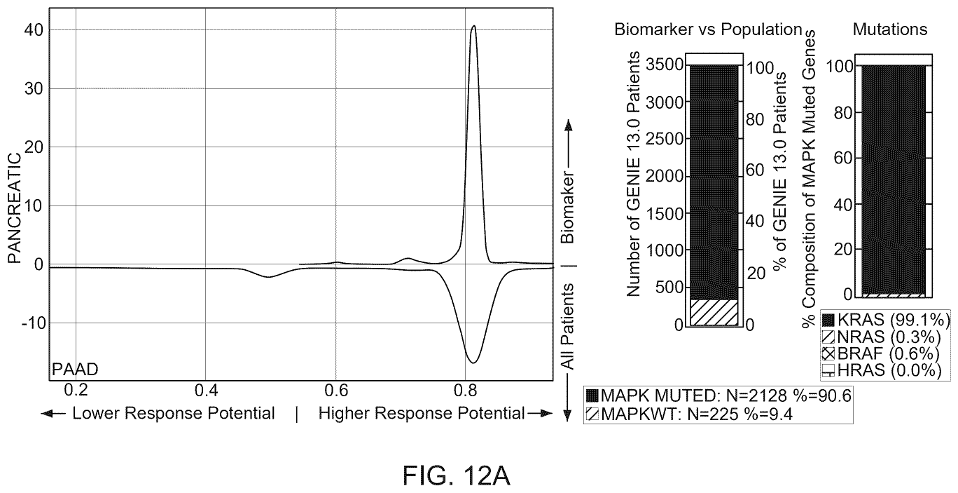

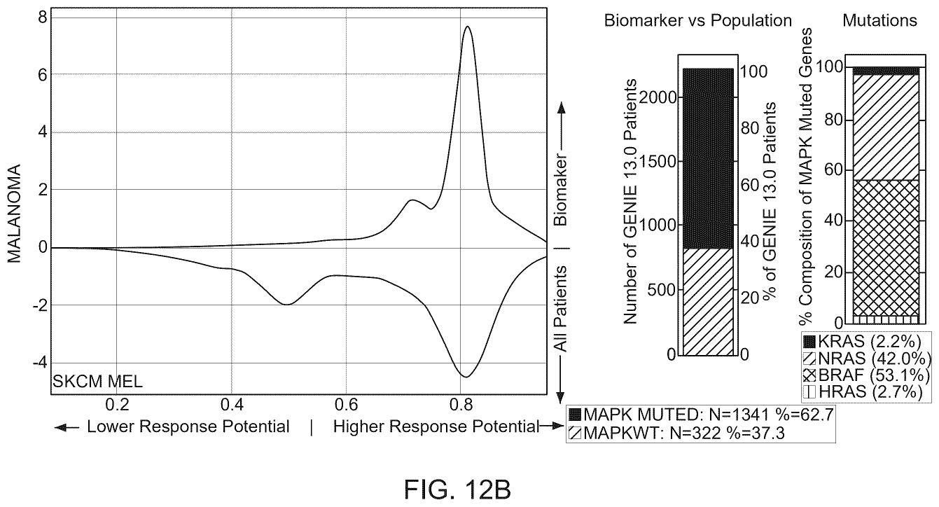

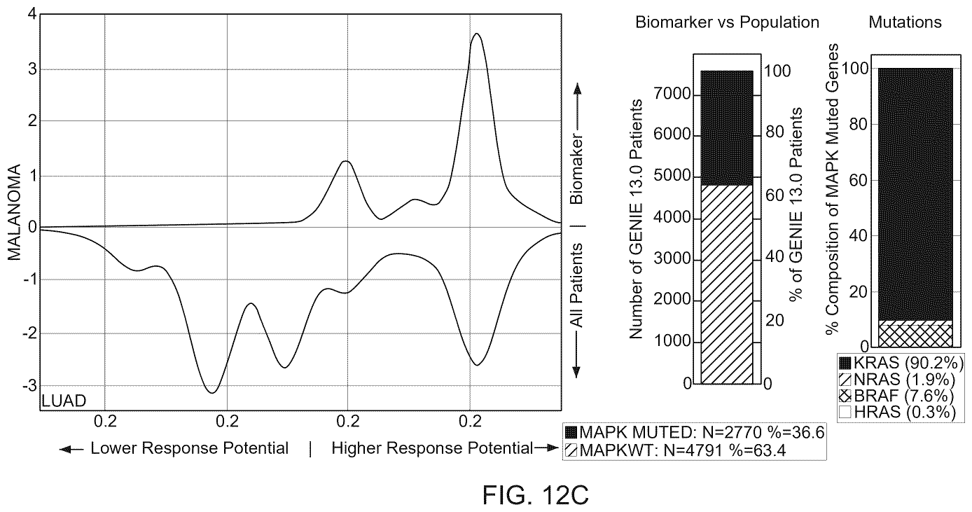

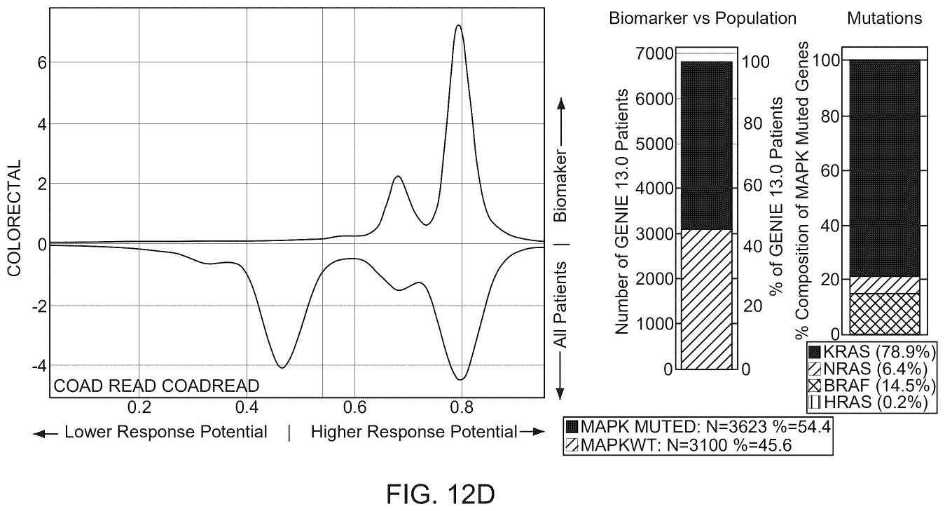

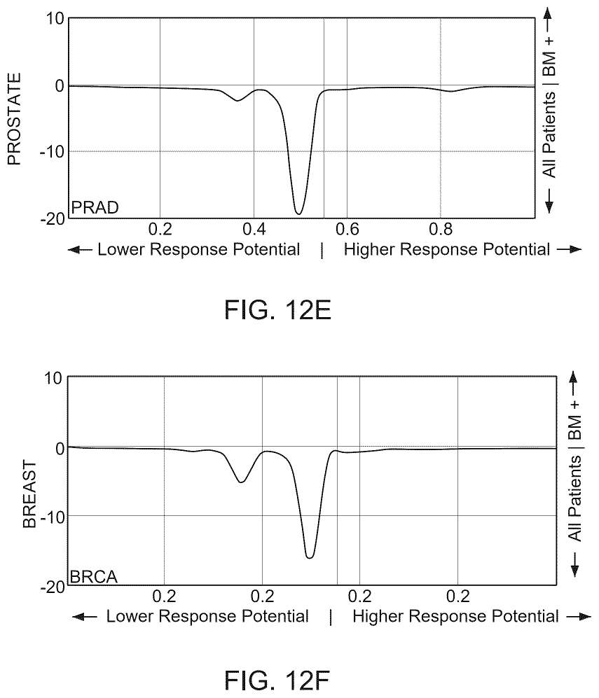

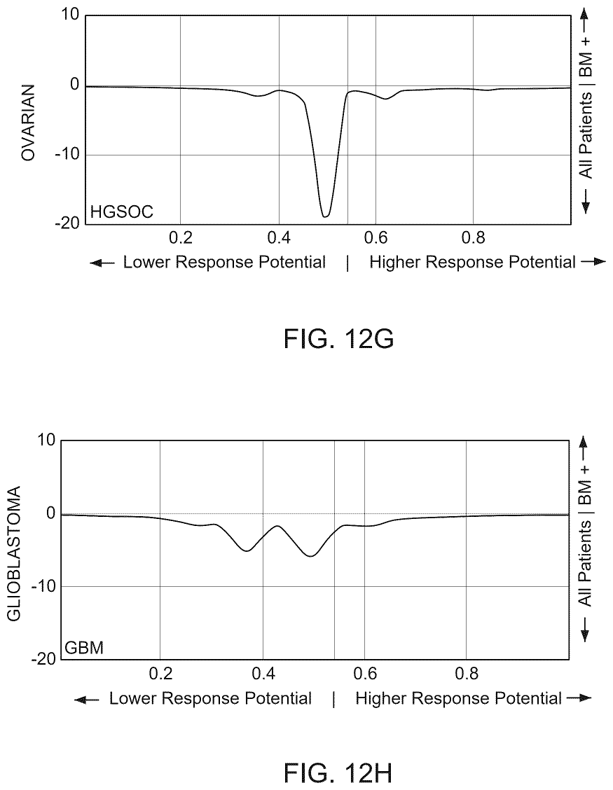

FIGS. 12A-12H show mapping of biomarker sensitive patient populations (GENIE) (MAPK+=GENIE patients per indication with activation mutation in KRAS, NRAS, HRAS, BRAF (class I or II)).

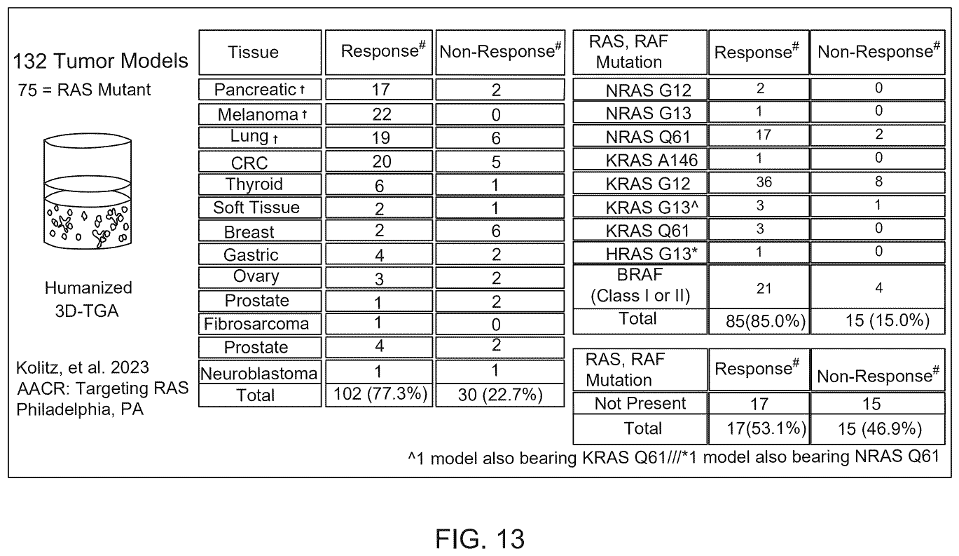

FIG. 13 shows tables of data showing the response to Compound A based on 3D-TGA and other preclinical modeling. Across all RAS-mutant models tested (132 tumor models, 75 of which have a reported RAS mutation), at least one model displayed response to Compound A for each observed RAS mutation, regardless of mutation position or amino acid substitution. No significant preference was observed with respect to response to Compound A across 30 KRAS G12 mutated cell lines, the most commonly mutated position in KRAS, from three major cancer indications including pancreatic, lung and colorectal cancer models. Parallel translational efforts were focused on projecting patient-aligned molecular profiles or ‘Targetability.’ # Models tested in 3D-TGA were assigned responsive if dose response IC50<1 μM (sensitive) or IC50≥1 with >25% reduction at 10 μM (intermediate), and non-responsive otherwise (resistant). † Selected 3D-TGA models: (1.) Pancreatic MIA PaCa-2 (sensitive/responsive), (2.) Pancreatic Capan-2 (intermediate/responsive), (3.) Melanoma SK-MEL-2 (sensitive/responsive), (4.) Lung A549 (intermediate/responsive).

### Example 11

### Oral Dosage Form of Compound A

An oral dosage form of Compound A can be prepared by combining Compound A as the active pharmaceutical ingredient (API) with pharmaceutically acceptable excipients to form a tablet. The intra granulation excipients can include microcrystalline cellulose (e.g., CEOLUS UF-711), crospovidone Type A (e.g., Polyplasdone XL), hydroxypropyl Cellulose HPC-SL, hydroxypropyl cellulose-Low Substituted L-HPC LH-21, and purified water. The extra granulation excipients can include Compound A and magnesium stearate (e.g., Hyqual™ vegetable source). For example, a tablet dosage form is described in the Table 4 below.

TABLE 4

| | |
|---|---|
| Ingredient | % w/w |

Intra Granulation

| | |
|---|---|
| Compound A (API) | 20.00 |
| Microcrystalline Cellulose | 64.25 |
| Crospovidone, Type A | 5.00 |
| Hydroxypropyl Cellulose HPC-SL | 5.00 |
| Hydroxypropyl Cellulose-Low Substituted, L-HPC LH-21 | 5.00 |
| Sub Total | 99.25 |

Extra Granulation

| | |
|---|---|
| Compound A (API) granulate | 99.25 |
| Magnesium Stearate | 0.75 |
| Sub Total | 100.00 |

### Example 12

### Human Clinical Trial of Compound A

A clinical trial with an open-label study designed to evaluate the safety, tolerability, clinical pharmacology, and preliminary efficacy of Compound A when dosed as monotherapy, once daily, in participants with advanced RAS mutant solid tumors, following at least one line of systemic, standard-of-care therapy was performed.

Initial phase 1/2a clinical data (NCT05585320) demonstrated favorable initial safety along with PK and PD profiles that were consistent with preclinical modeling of deep cyclic inhibition (DCI), including Cmax levels over 2,000 ng/mL (~1 μM drug FF), a median plasma t_{1/2} of 1.94 hours, and ~90% pharmacodynamic inhibition of pERK.

The study population comprised male and female participants 18 years of age or older with advanced, unresectable, or metastatic RAS-mutated solid tumors who had received at least 1 prior line of systemic therapy for their advanced/metastatic disease. Patients for treatment had any advanced, unresectable, or metastatic RAS-mutated solid tumors in Phase 1, while Phase 2a will focus on tumor type cohorts: NRAS-mutated melanoma, KRAS-mutated PDAC, KRAS-mutated NSCLC and KRAS-mutated, APC-wildtype CRC.

The cohort dose escalation levels included oral administration of Compound A at doses of 40, 80, 160, 320 mg QD, for up to twelve 28-day cycles. Once daily administration allowed for more than 10 half-lives between doses. Timed serial PK samples were collected on C1D1 and C1D15 and trough PK samples were collected at C1D2, C1D8, C1D16, C2D1 and C3D1.

Table 5 provides clinical data obtained from initial pharmacokinetics (PK) and pharmacodynamics (PD) data from the ongoing phase 1/2a trial (NCT05585320), from certain human subjects enrolled during the first 20 weeks since the first patient was dosed in this Phase 1 clinical trial, showing the calculated half-life based on samples taken at cycle 1 day 1 (C1D1) and cycle 1, day 15 (C1D15), after receiving once-daily oral doses of Compound A at doses of 40 mg, 80 mg, or 160 mg. Each patient was diagnosed with the RAS-mutant cancer diagnosis indicated in Table 5 prior to treatment with Compound A. No dose limiting toxicities (DLT) or severe adverse events (SAE) were observed while dosing these patients through C1D15. No Compound A drug-related adverse events beyond grade 1 were reported in dose levels III or IV. The clinical trial is actively enrolling patients at 320 mg QD p.o. with 2 additional patients already consented (KRAS-G12V pancreatic cancer and KRAS-G12D colorectal cancer). No dose limiting toxicities (DLTs) or serious adverse events (SAEs) were observed in the first 20 weeks since dosing the first patient. Early PK data are approximately dose linear with no Compound A drug accumulation.

TABLE 5
Patient Status Summary for Compound A Phase 1 Clinical Trial

| | | RAS | Dose | | C1D1 | C1D15 | Mean | Cmpd A |
|---|---|---|---|---|---|---|---|---|
| # | Patient Cancer | Mutation | Level | Dose | (t_{1/2}) | (t_{1/2}) | (t_{1/2}) | Status |
| 1. | PANCREATIC | KRAS- | I | 40 mg | 1.82 | 2.10 | 1.96 | Off |
| | | G12D | | QD p.o. | hours | hours | hours | Treatment |
| 2. | COLORECTAL | KRAS- | II | 80 mg | 1.41 | 1.43 | 1.42 | Off |
| | | G12V | | QD p.o. | hours | hours | hours | Treatment |
| 3. | COLORECTAL | NRAS- | III | 160 mg | 2.04 | 1.83 | 1.94 | Off |
| | | Q61L | | QD p.o. | hours | hours | hours | Treatment |
| 4. | COLORECTAL | NRAS- | III | 160 mg | 1.91 | 1.97 | 1.94 | On |
| | | Q61K | | QD p.o. | hours | hours | hours | Treatment |
| 5. | PANCREATIC | KRAS- | IV | 320 mg | 2.31 | 2.46 | 2.38 | On |
| | | G12D | | QD p.o. | hours | hours | hours | Treatment |
| 6. | PANCREATIC | KRAS- | IV | 320 mg | 2.04 | 2.27 | 2.16 | On |
| | | G12V | | QD p.o. | hours | hours | hours | Treatment |
| 7. | COLORECTAL | KRAS- | IV | 320 mg | 1.45 | 2.27 | 1.86 | On |
| | | G12S | | QD p.o. | hours | hours | hours | Treatment |

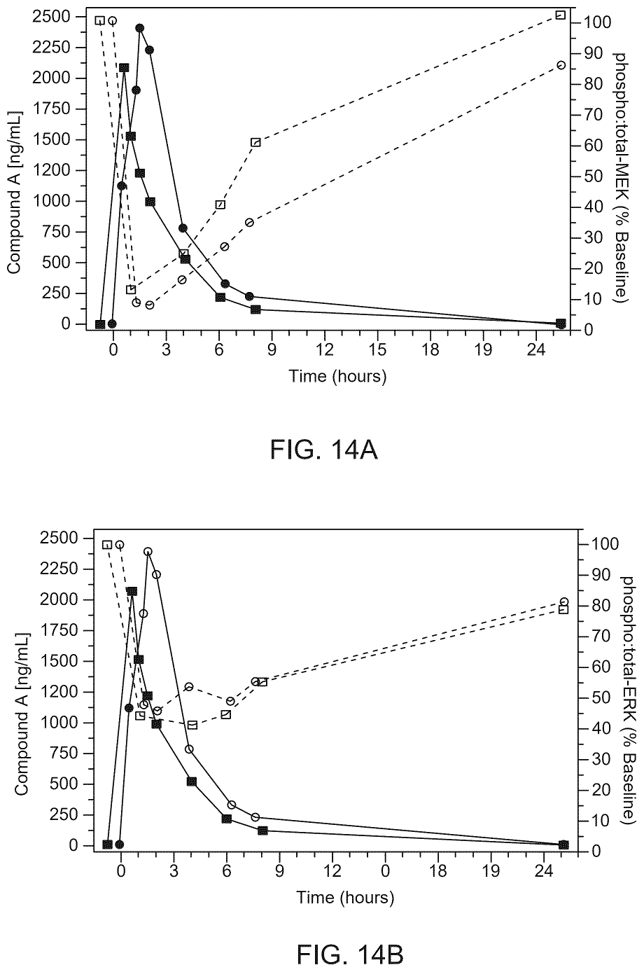

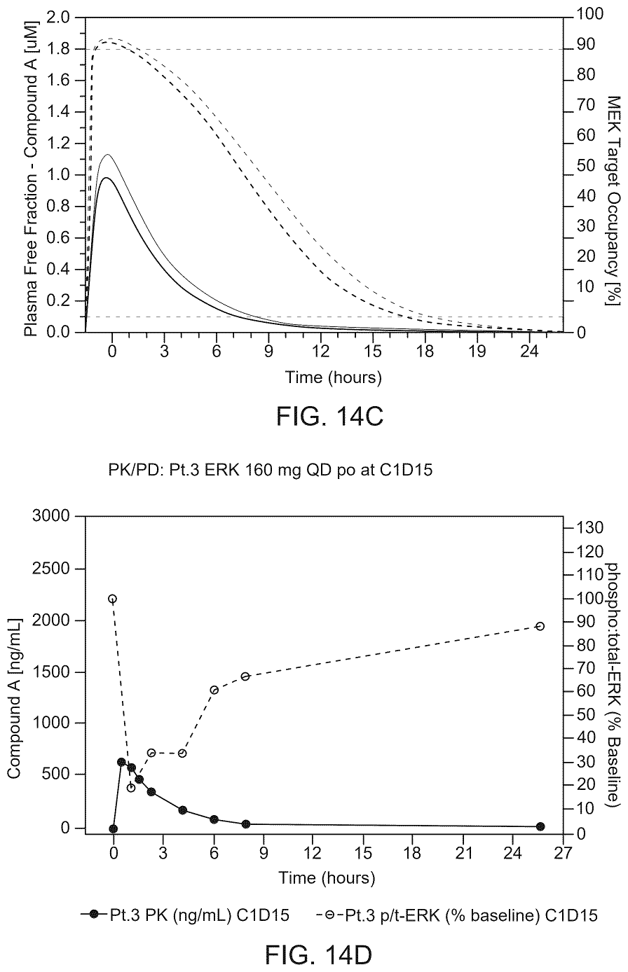

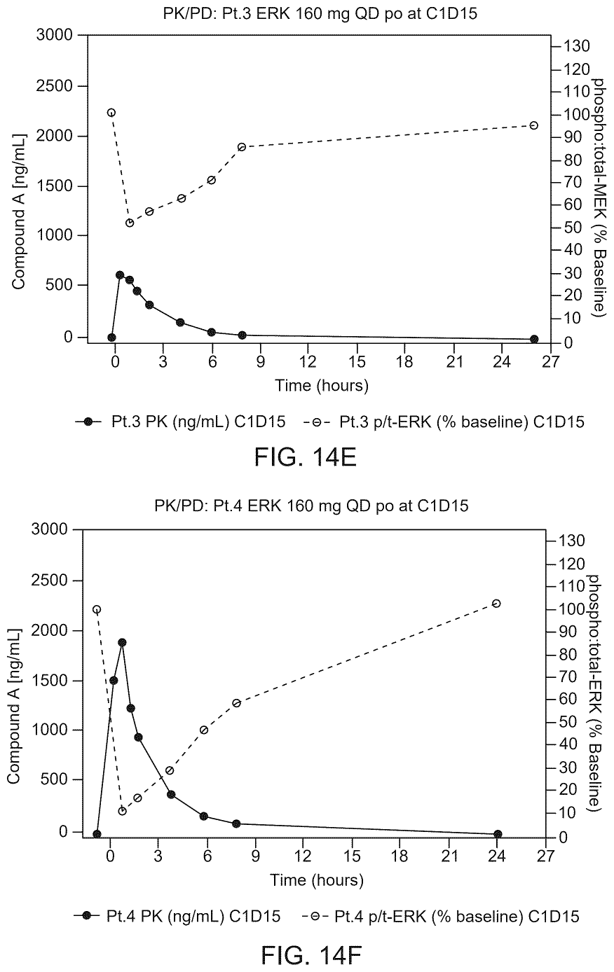

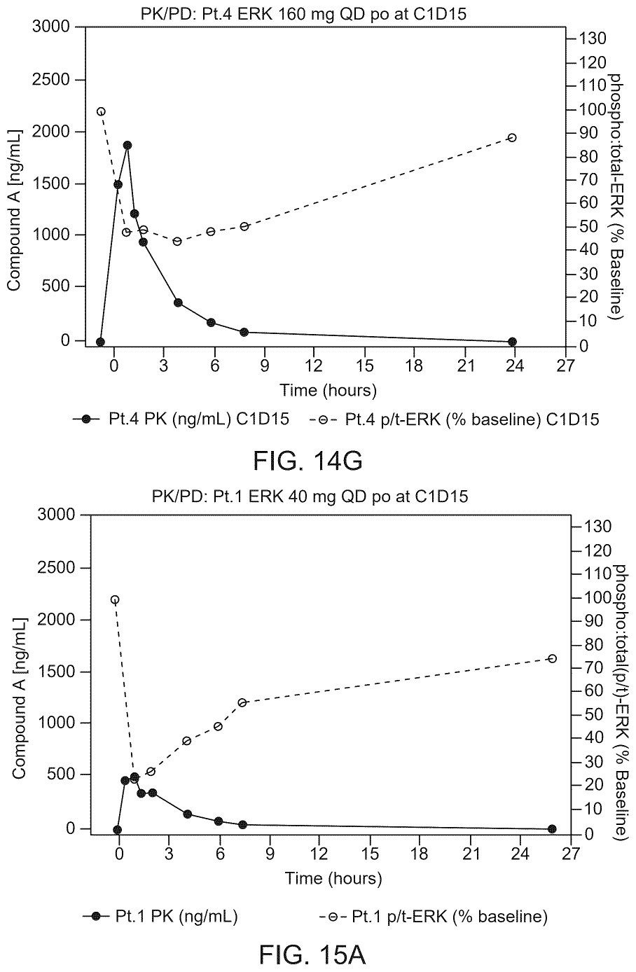

In patients 3 and 4, an approximately 90% pharmacodynamic (PD) inhibition of pERK was observed in patients receiving 160 mg of Compound A administered orally (po) once daily (QD). (Dose Level III, cycle 1, day 1). FIG. 14A shows Compound A PK/PD (pERK) showing MAPK pathway suppression (87-92% reduction p/t-ERK, Cmax≥2,000 ng/mL and Cmin of about 0.1 micromolar free fraction 6-8 hours). FIG. 14B shows Compound A PK/PD (phosphorylated MEK) data showing suppression of pathway bypass (55-59% reduction p/total-MEK, Cmax≥2,000 ng/mL and Cmin of about 0.1 micromolar free fraction 6-8 hours). FIG. 14C shows modeled PK/BK, drug free fraction (FF) with a high Cmax (see Hoare, Sam R J. “Analyzing kinetic binding data.” Assay Guidance Manual) (90% MEK occupancy, Cmax 1 micromolar FF, Cmin of about 0.1 micromolar free fraction 6-8 hours). Referring to FIG. 14A-C:

Dose Level III: Cycle 1 Day 1 (C1D1) PK (solid line), BK (dotted line), and PD (dotted line) for patient 3 (solid line with circles) and 4 (solid line with solid squares), both at 160 mg QD p.o.

PK plasma: pre-dose (0), 0.5, 1, 1.5, 2*, 4, 6, 8, 24 hours; PD plasma: PK-matched without 0.5 or 1.5 hours (*poor sample quality for Pt.4 at 2 hour)

PD method: A549 (KRASG12S) cells were exposed to patient plasma for 2-hours before quantifying phosphorylated and total ERK and MEK

FIG. 14A is a graph showing PK/PD (pERK) for Compound A from patients 3 and 4 after receiving 160 mg Compound A QD po (cycle 1, day 1), showing Compound A (pERK) MAPK pathway suppression. FIG. 14B is a graph showing PK/PD (pMEK) for Compound A from patients 3 and 4 after receiving 160 mg Compound A QD po (cycle 1, day 1), shows suppression of pathway bypass. FIG. 14C is a graph showing the modeled PK/BK based on data from patients 3 and 4 after receiving 160 mg Compound A QD po (cycle 1, day 1), drug free fraction (FF) for Compound A including a high Compound A Cmax.

FIG. 14D is a graph showing PK/PD (pERK) for Compound A from patient 3 after receiving 160 mg Compound A QD po (cycle 1, day 15). FIG. 14E is a graph showing PK/PD (pMEK) for Compound A from patient 3 after receiving 160 mg Compound A QD po (cycle 1, day 15). FIG. 14F is a graph showing PK/PD (pERK) for Compound A from patient 4 after receiving 160 mg Compound A QD po (cycle 1, day 15). FIG. 14G is a graph showing PK/PD (pMEK) for Compound A from patient 4 after receiving 160 mg Compound A QD po (cycle 1, day 15).

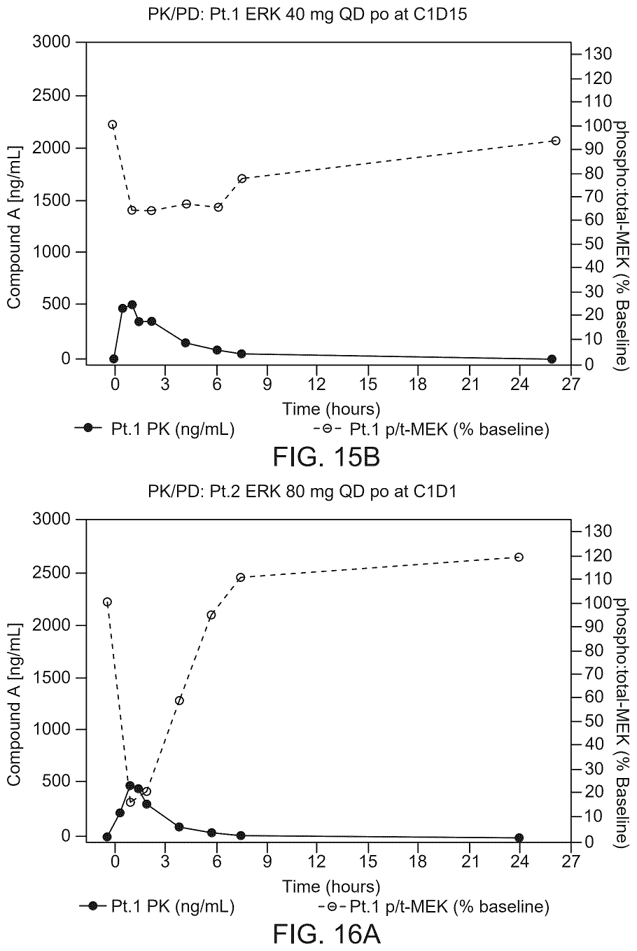

FIG. 15A is a graph showing PK/PD (pERK) for Compound A from patient 1 after receiving 40 mg Compound A QD po (cycle 1, day 1). FIG. 15B is a graph showing PK/PD (pMEK) for Compound A from patient 1 after receiving 40 mg Compound A QD po (cycle 1, day 1).

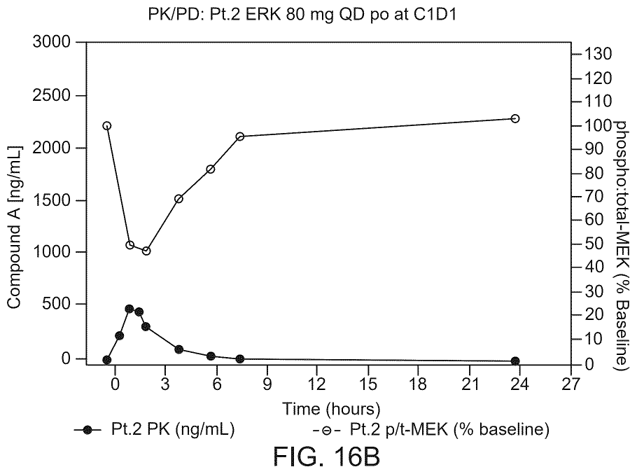

FIG. 16A is a graph showing PK/PD (pERK) for Compound A from patients after receiving 80 mg Compound A QD po (cycle 1, day 1). FIG. 16B is a graph showing PK/PD (pMEK) for Compound A from patient 2 after receiving 80 mg Compound A QD po (cycle 1, day 1).

Significant PK Cmax levels (plasma concentration of therapy in a specific area of the body) were observed with Compound A of over 2,000 ng/mL (or approximately 1 μM drug free-fraction at 160 mg once daily oral dose)

Greater than 90 percent PD inhibition of phosphorylated extracellular signal-regulated kinase (pERK) with Compound A compared to pretreatment baseline for patients at the third dose level (160 mg once daily oral)

A median plasma half-life (t_{1/2}) of 1.94 hours observed with Compound A across the first three dose levels evaluable (40 mg, 80 mg and 160 mg once daily oral), in patients with pancreatic and colorectal cancer with different RAS mutations, including KRAS-G12D, the most common mutation presents in pancreatic cancer.

Compound A was well tolerated with no DLTs or SAEs observed and no drug-related adverse events beyond Grade 1 observed.

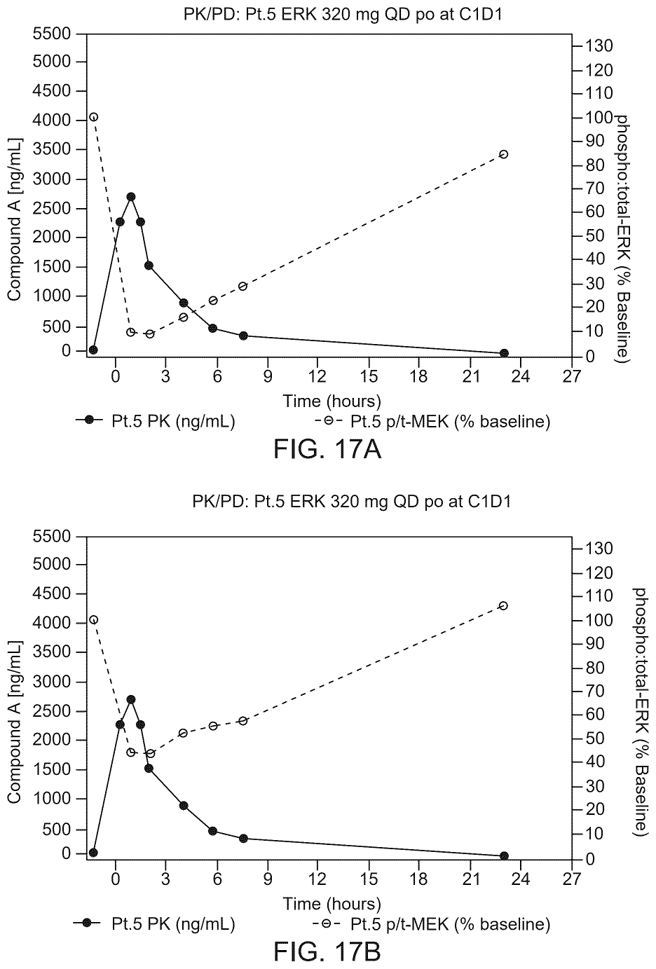

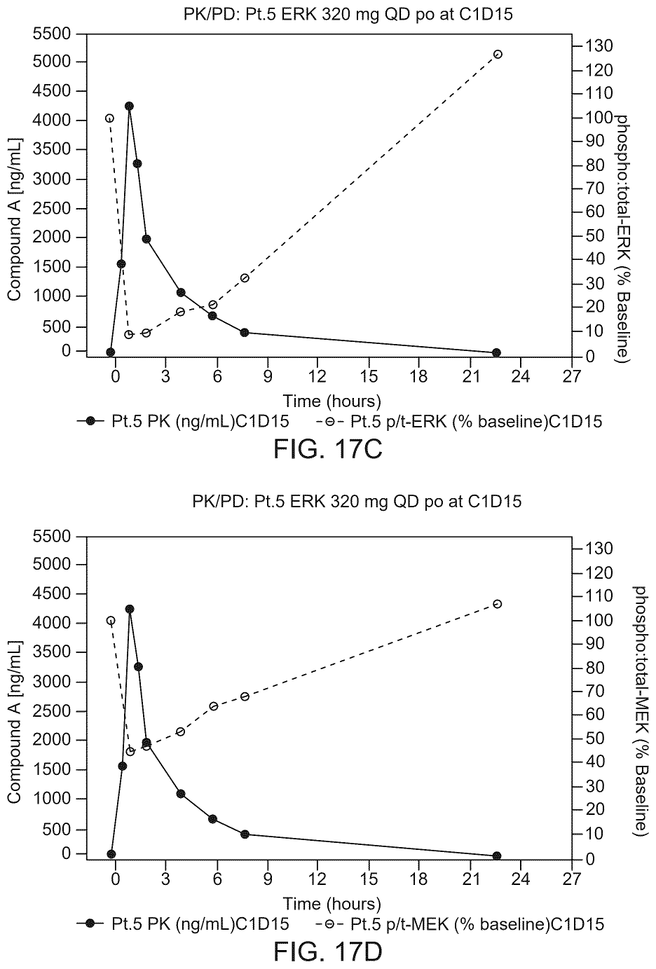

Patient 5 was diagnosed with KRAS-G12D pancreatic cancer and received 320 mg of Compound A once daily, for 15 days in cycle 1. Table 5 provides the corresponding half-life measured at day 1 and day 15. FIG. 17A is a graph showing PK/PD (pERK) for Compound A from patient 5 after receiving 320 mg Compound A QD po (cycle 1, day 1). FIG. 17B is a graph showing PK/PD (pMEK) for Compound A from patient 5 after receiving 320 mg Compound A QD po (cycle 1, day 1). FIG. 17C is a graph showing PK/PD (pERK) for Compound A from patient 5 after receiving 320 mg Compound A QD po (cycle 1, day 15). FIG. 17D is a graph showing PK/PD (pMEK) for Compound A from patient 5 after receiving 320 mg Compound A QD po (cycle 1, day 15).

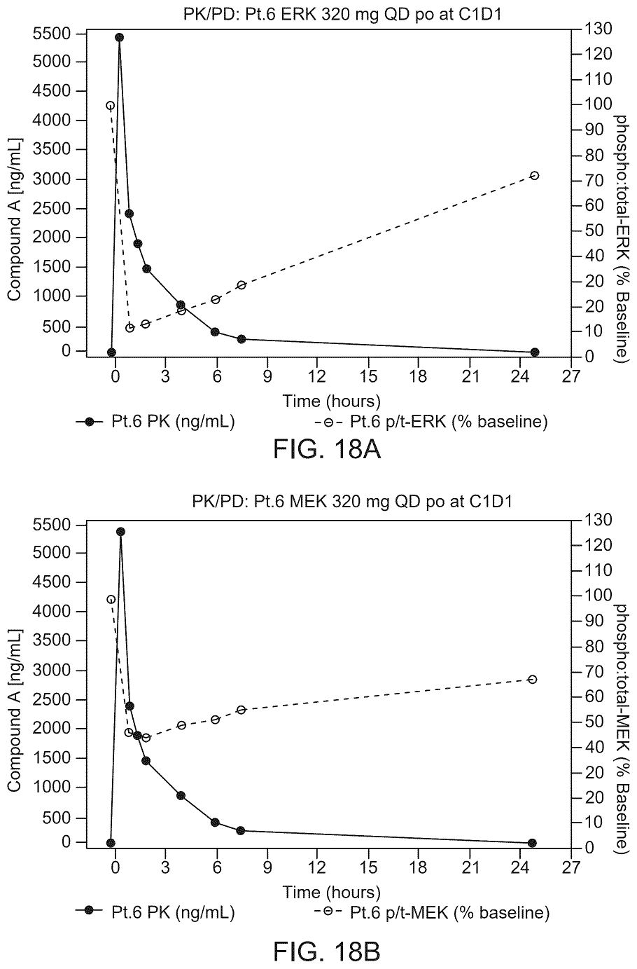

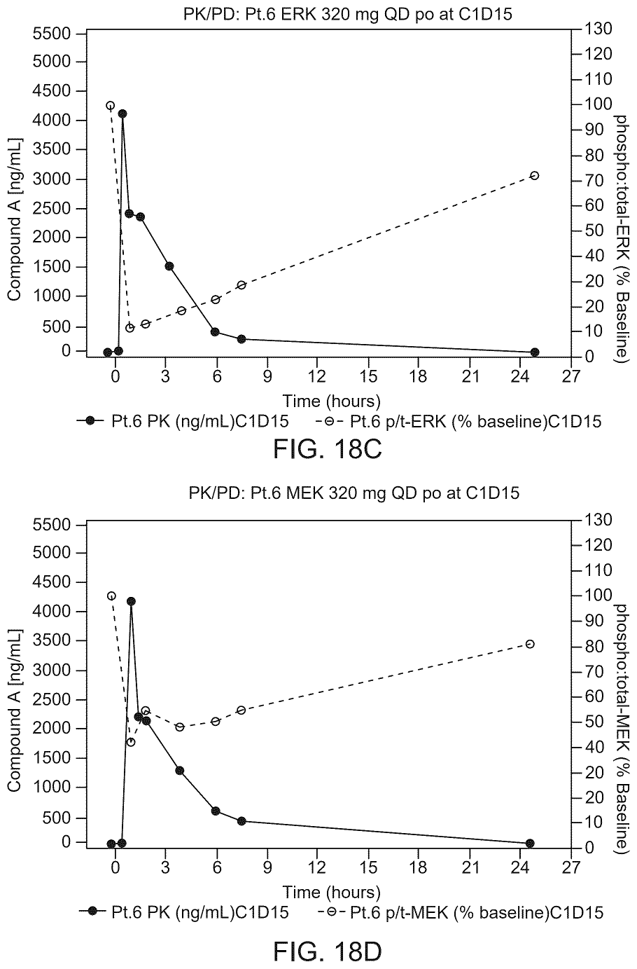

Patient 6 was diagnosed with KRAS-G12V pancreatic cancer and received 320 mg of Compound A once daily, for 15 days in cycle 1. Table 5 provides the corresponding half-life measured at day 1 and day 15. FIG. 18A is a graph showing PK/PD (pERK) for Compound A from patient 6 after receiving 320 mg Compound A QD po (cycle 1, day 1). FIG. 18B is a graph showing PK/PD (pMEK) for Compound A from patient 6 after receiving 320 mg Compound A QD po (cycle 1, day 1). FIG. 18C is a graph showing PK/PD (pERK) for Compound A from patient 6 after receiving 320 mg Compound A QD po (cycle 1, day 15). FIG. 18D is a graph showing PK/PD (pMEK) for Compound A from patient 6 after receiving 320 mg Compound A QD po (cycle 1, day 15).

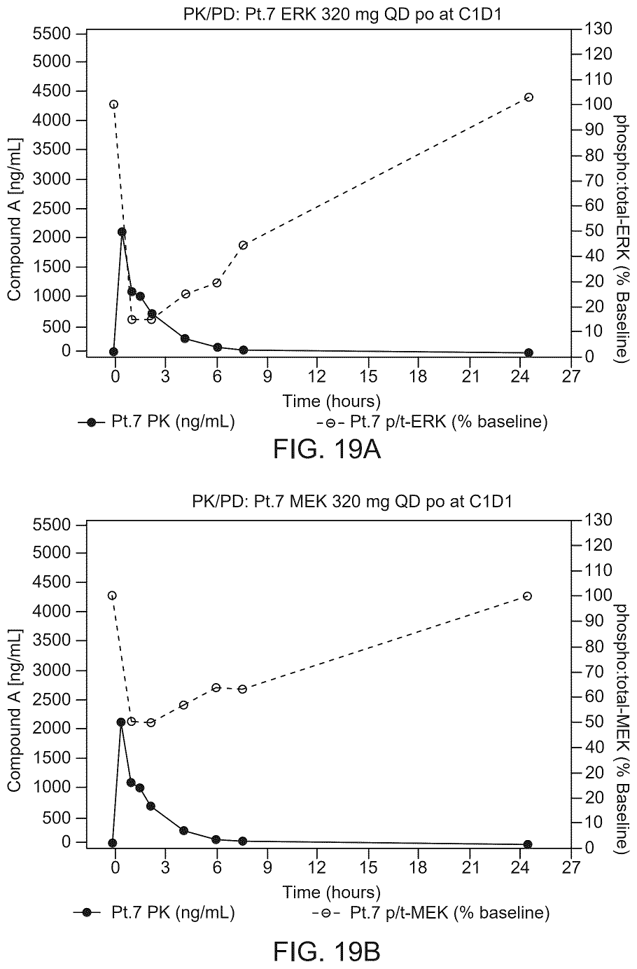

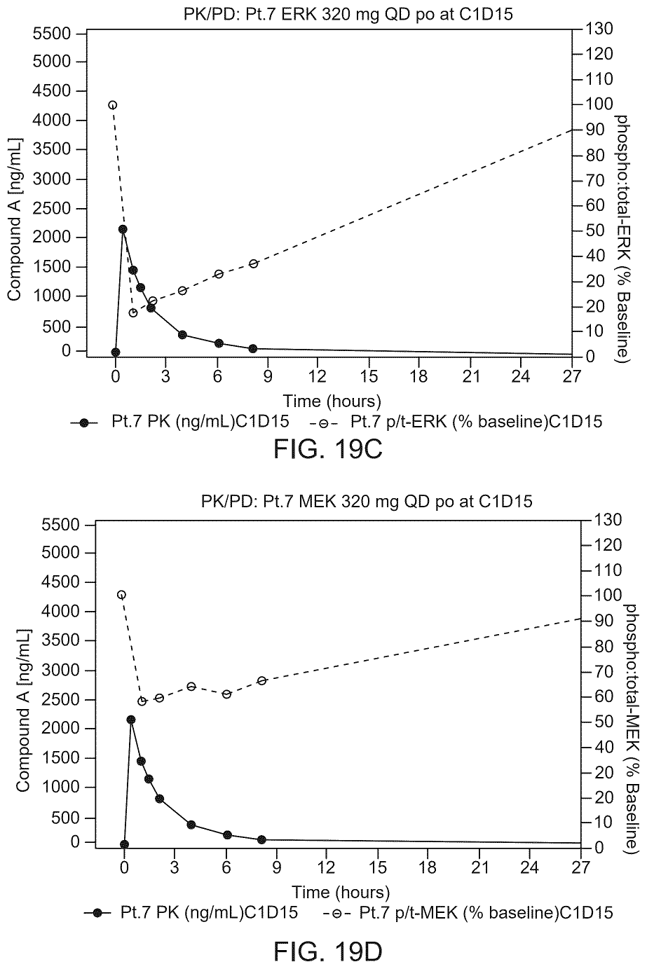

Patient 7 was diagnosed with KRAS-G12S colorectal cancer and received 320 mg of Compound A once daily, for 15 days in cycle 1. Table 5 provides the corresponding half-life measured at day 1 and day 15. FIG. 19A is a graph showing PK/PD (pERK) for Compound A from patient 7 after receiving 320 mg Compound A QD po (cycle 1, day 1). FIG. 19B is a graph showing PK/PD (pMEK) for Compound A from patient 7 after receiving 320 mg Compound A QD po (cycle 1, day 1). FIG. 19C is a graph showing PK/PD (pERK) for Compound A from patient 7 after receiving 320 mg Compound A QD po (cycle 1, day 15). FIG. 19D is a graph showing PK/PD (pMEK) for Compound A from patient 7 after receiving 320 mg Compound A QD po (cycle 1, day 15).

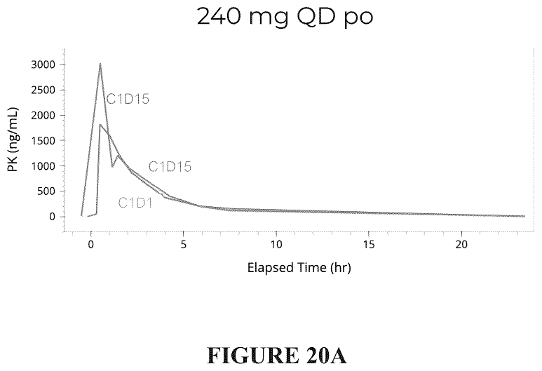

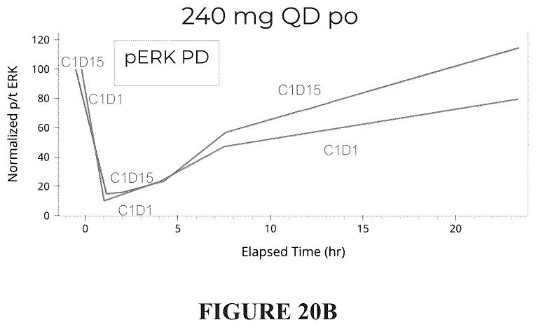

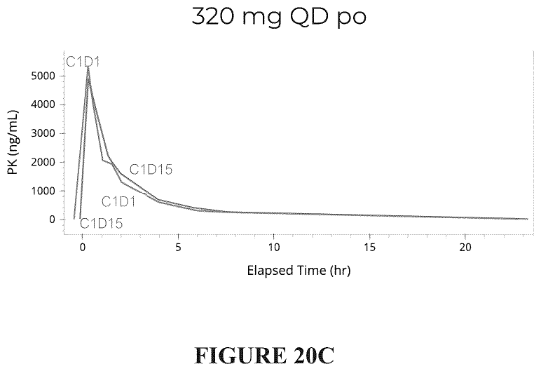

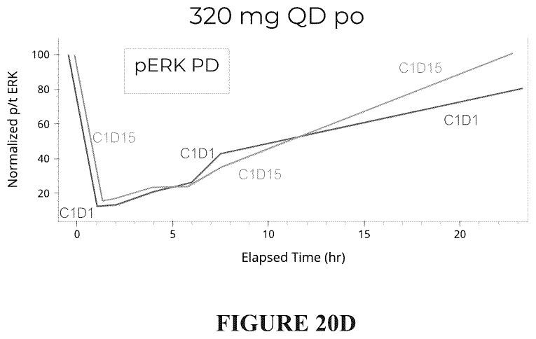

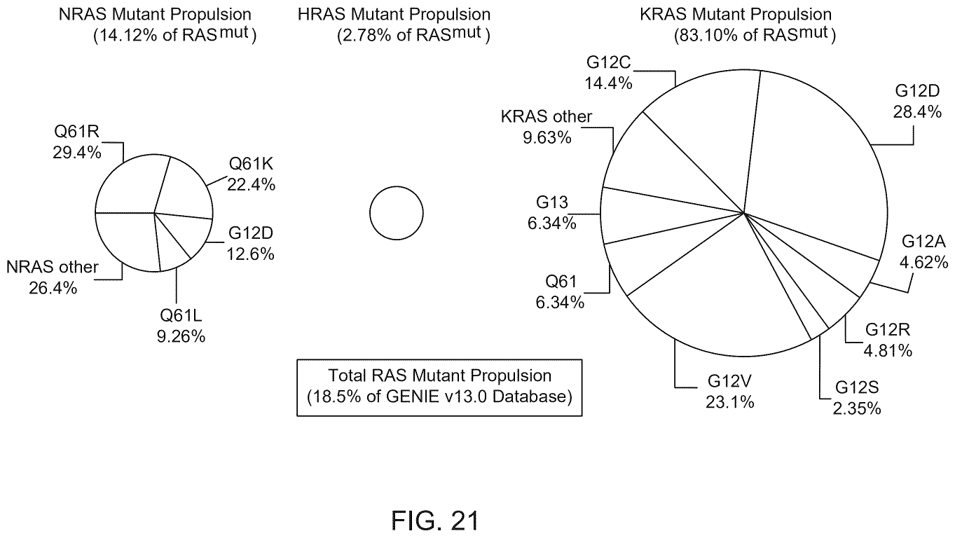

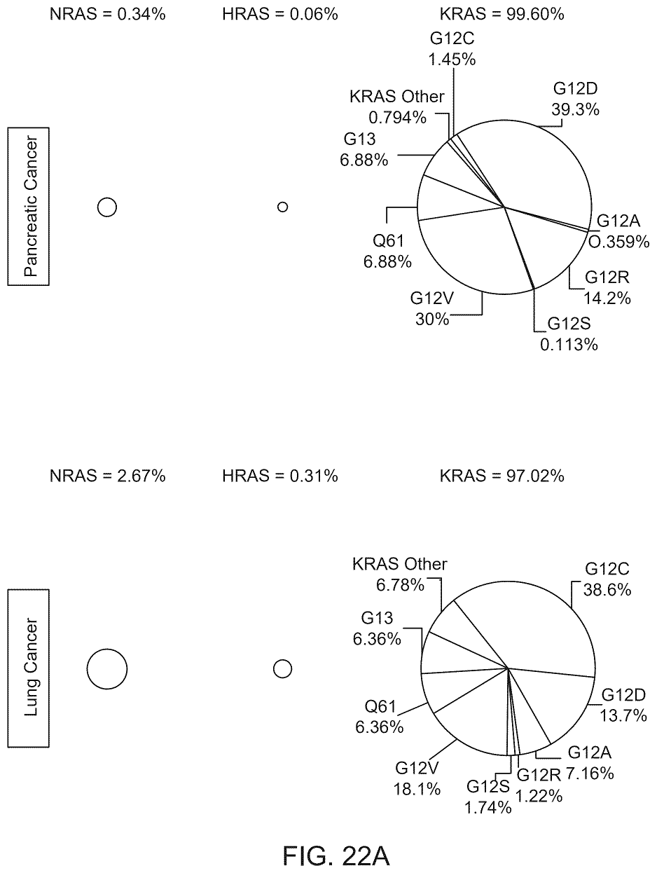

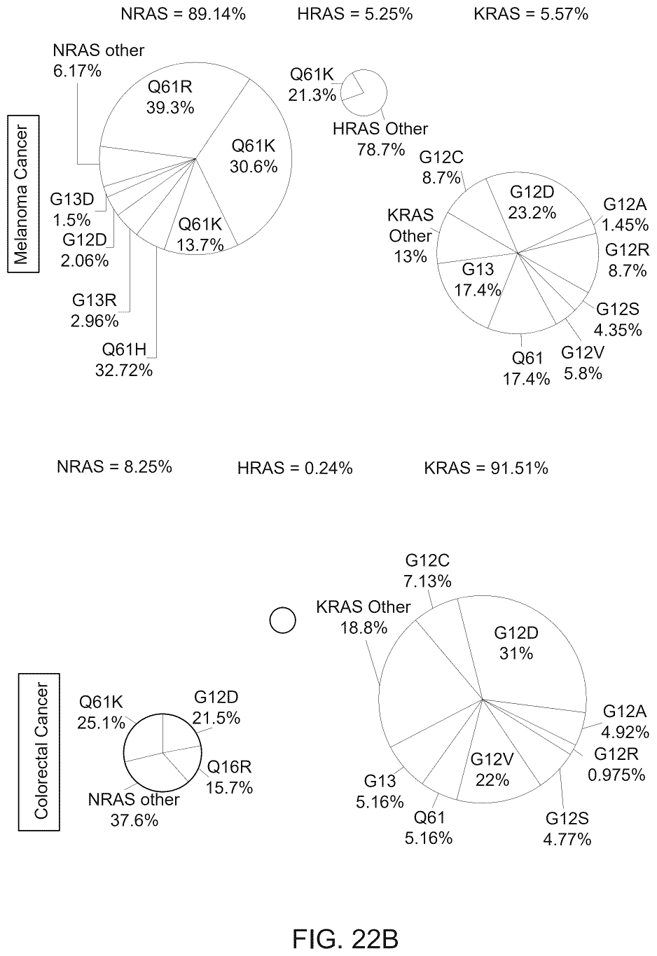

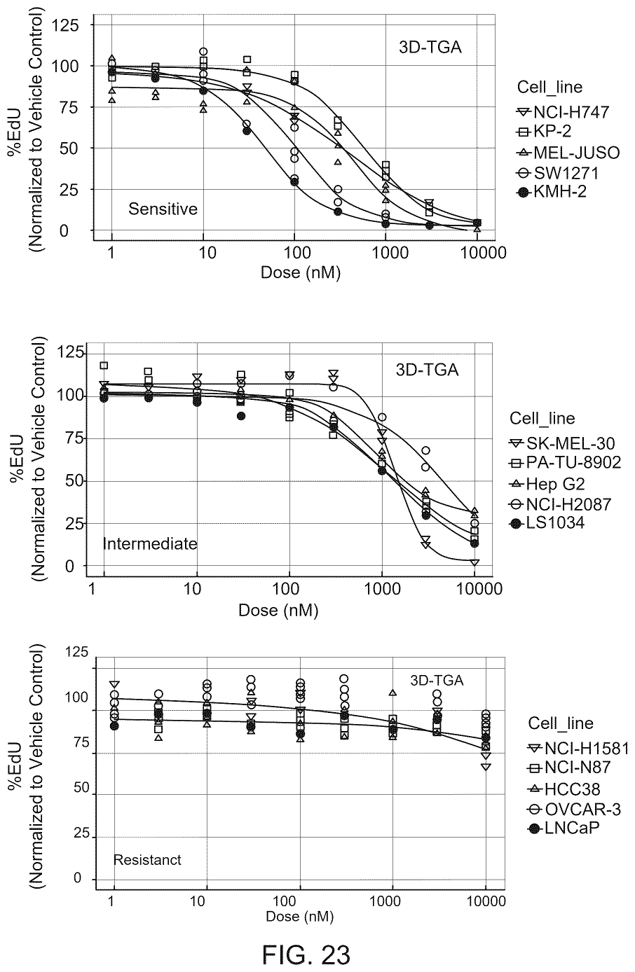

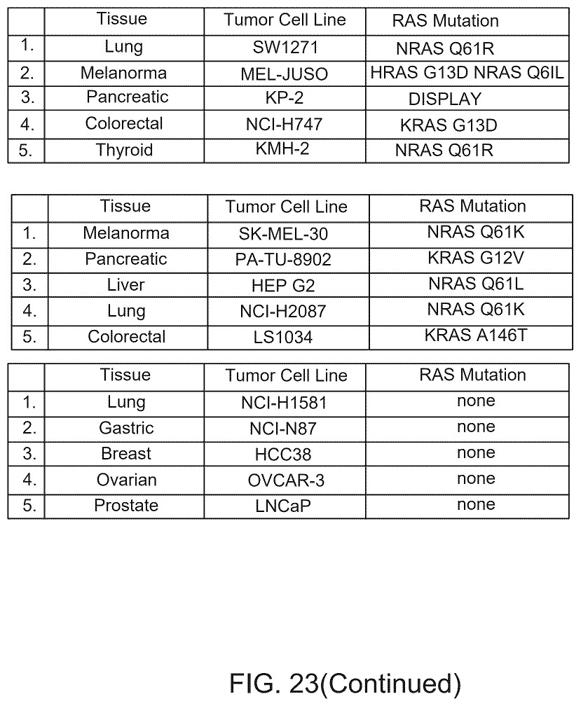

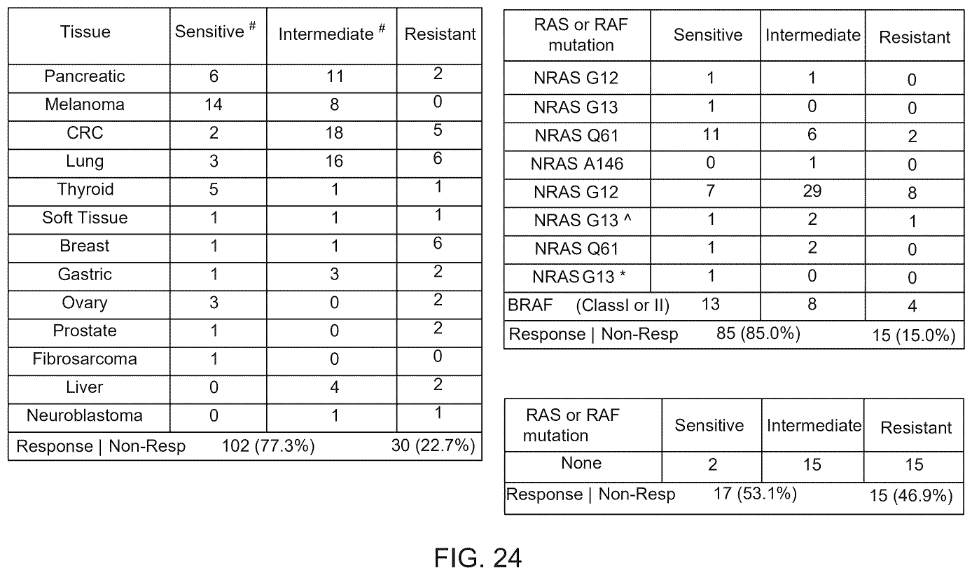

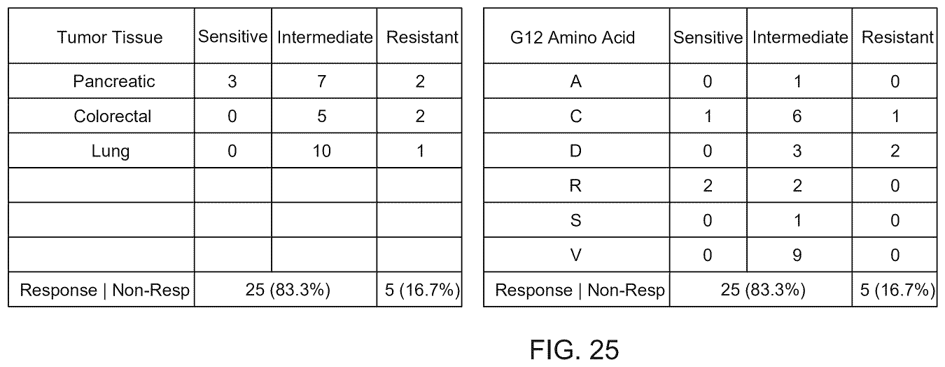

FIG. 20A is a graph showing plasma concentration (PK) for Compound A from patient 13 after receiving 240 mg Compound A administered orally (po) once daily (QD) at cycle 1, day 1 (grey line) and at cycle 1, day 15 (blue line). FIG. 20B is a graph showing PK/PD (pERK) for Compound A from patient 13 after receiving 240 mg Compound A administered orally (po) once daily (QD) at cycle 1, day 1 (blue line) and at cycle 1, day 15 (red line).

FIG. 20C is a graph showing plasma concentration (PK) for Compound A from patient 16 after receiving 320 mg Compound A administered orally (po) once daily (QD) at cycle 1, day 1 (blue line) and at cycle 1, day 15 (red line). FIG. 20D is a graph showing PK/PD (pERK) for Compound A from patient 16 after receiving 320 mg Compound A administered orally (po) once daily (QD) at cycle 1, day 1 (blue line) and at cycle 1, day 15 (purple line).

### Example 13

### Human Clinical Trial of Compound A

A clinical trial with an open-label study designed to evaluate the safety, tolerability, clinical pharmacology, and preliminary efficacy of Compound A when dosed as monotherapy, once daily, in participants with advanced RAS mutant solid tumors, following at least one line of systemic, standard-of-care therapy was performed.

Initial phase 1/2a clinical data (NCT05585320) demonstrated favorable initial safety along with PK and PD profiles that were consistent with preclinical modeling of deep cyclic inhibition (DCI), including Cmax levels over 2,000 ng/mL (~1 uM drug FF), a median plasma t½ of 1.94 hours, and ~90% pharmacodynamic inhibition of pERK.

The study population comprised male and female participants 18 years of age or older with advanced, unresectable, or metastatic RAS-mutated solid tumors who have received at least 1 prior line of systemic therapy for their advanced/metastatic disease. Patients for treatment were selected such that they may have any advanced, unresectable, or metastatic RAS-mutated solid tumors in Phase 1, while Phase 2a focused on tumor type cohorts: NRAS-mutated melanoma, KRAS-mutated PDAC, KRAS-mutated NSCLC and KRAS-mutated, APC-wildtype CRC.

The cohort dose escalation levels included oral administration of Compound A at a dose of 240 mg QD, for up to twelve 28-day cycles. Once daily administration allowed for more than 10 half-lives between doses. Timed serial PK samples were collected on C1D1 and C1D15 and trough PK samples are collected at C1D2, C1D8, C1D16, C2D1 and C3D1.

This is an example of developing a medicine for a broad population of cancer patients using a universal-RAS therapy in the dose-escalation portion of the Phase 1/2a study for Compound A in patients with advanced solid tumors. The study's Safety Review Committee completed its evaluation and determined that doses up to and including 320 mg (including, for example, 240 mg) once daily were tolerable with no dose limiting toxicities. Enrollment in the Phase 1b expansion portion of the study will commence and is designed to evaluate two dosing cohorts of 12 patients each at an oral dose of 240 mg or 320 mg once daily, with three patients already in the study have so far been dosed at the 320 mg dose level.

This unique therapy aims for Universal-RAS activity: treating patients with any mutation in KRAS, NRAS, or HRAS. As such, there are plans to enroll 21 additional patients in next part of the study to materially add to a dataset for Compound A in patients with RAS-mutant solid tumors.

Compound A achieved significant levels of PK Cmax, demonstrated a half-life of approximately two hours, and was well tolerated with no dose limiting toxicities or serious adverse events observed in patients with pancreatic and colorectal cancer. Upon review of the cumulative data from the eight patients dosed in the Phase 1a study, the SRC endorsed 240 mg daily and 320 mg daily as the dose levels for the expansion cohort and agreed with initiating the Phase 1b expansion.

Based on these results, it is believed that Compound A may achieve universal-RAS activity that selectively impacts cancer cells to a greater extent than healthy cells, through deep cyclic inhibition of the MAPK pathway with once-daily oral dosing. Compound A continues to be evaluated in a Phase 1/2a study in patients with advanced solid tumors harboring RAS mutations for whom there are limited treatment options (NCT05585320).

### Example 14

### Comparative Physicochemical Properties

Certain physiochemical properties of Compound A are provided in Tables 6A-6E below in comparison to certain comparator control compounds and reference compounds. Reference 1 is Selumetinib.

### Reference 2 is

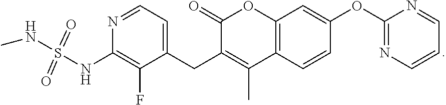 [SMILES: CNS(=O)(=O)Nc1nccc(Cc2c(C)c3ccc(Oc4ncccn4)cc3oc2=O)c1F]

### Reference 7 is

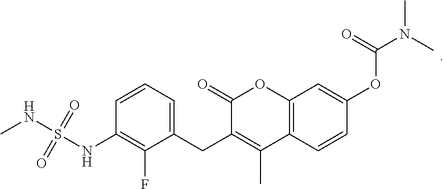

TABLE 6A

| Compound | Mean Pₐₚₚ | Mean Pₐₚₚ | Mean | A − B |
|---|---|---|---|---|
| (10 μM; | A − B | B − A | (B − A/ | Perme- |
| Caco-2; | (10⁻⁶ | (10⁻⁶ | A − B) | ability |
| 2 hrs) | cm/s) | cm/s) | (Efflux Ratio)\hat{ } | Ranking* |
| Compound 9 | 13.0 | 21.0 | 1.62 | High |
| (Compound A) | | | | |
| Reference-1 | 8.83 | 28.2 | 3.19 | High |
| Reference-2 | 18.1 | 20.9 | 1.16 | High |
| Reference-7 | 14.4 | 10.0 | 0.695 | High |

Controls

| | | | | |
|---|---|---|---|---|
| Ranitidine | 0.347 | 1.41 | 4.06 | Low |
| Talinolal | 0.375 | 8.20 | 21.8 | Effluxed |
| Talinolal + | 0.766 | 3.07 | 4.01 | Inhibited |
| Verapamil | | | | Efflux |
| (2.5 μM) | | | | |
| Warfarin | 36.6 | 26.6 | 0.726 | High |

TABLE 6B
Physicochemical Properties: hERG Channel

| Compound | hERG IC50 |
|---|---|
| (30 to 0.01 μM dose range) | [μM] |
| Reference-1 | >30 |
| Reference-2 | >30 |
| Reference-7 | 28.6 |
| Compound 9 | 26.2 |
| (Compound A) | |

Controls

| | |
|---|---|
| Cisapride | 0.01 |

TABLE 6C
Physicochemical Properties: Microsome Stability

| | Human | Beagle Dog | SD Rat | CD-1 Mouse |
|---|---|---|---|---|
| | (% | (% | (% | (% |
| Compound | Reamining | Reamining | Remaining | Reamining |
| [2 μM] | @1 hour) | @1 hour) | @1 hour) | @1 hour) |
| Reference-1 | 75.8 | 97.5 | 54.6 | 13.9 |
| Reference-2 | 107.1 | 111.6 | 102.0 | 96.0 |
| Reference-7 | 61.1 | 57.0 | 2.7 | 29.6 |
| Compound 9 | 51.7 | 76.5 | 28.8 | 54.3 |
| (Compound A) | | | | |

Controls

| | | | | |
|---|---|---|---|---|
| Verapamil | 5.8 | 6.0 | 0.7 | 0.8 |

TABLE 6D
Physicochemical Properties: Plasma Stability

| | Human | Beagle Dog | SD Rat | CD-1 Mouse |
|---|---|---|---|---|
| | (% | (% | (% | (% |
| Compound | Reamining | Reamining | Remaining | Reamining |
| [2 μM] | @2 hour) | @2 hour) | @2 hour) | @2 hour) |
| Reference-1 | 98.1 | 100.4 | 94.9 | 104.8 |
| Reference-2 | 91.1 | 99.8 | 99.8 | 92.9 |
| Reference-7 | 88.1 | 99.8 | 77.3 | 56.5 |
| Compound 9 | 95.1 | 96.8 | 106.4 | 90.6 |
| (Compound A) | | | | |

Controls

| | | | | |
|---|---|---|---|---|
| Propantheline | 5.7 | 58.4 | 92.6 | 27.1 |
| Lovastatin | 81.8 | 94.1 | 0.6 | 0.6 |

TABLE 6E
Physicochemical Properties: CYP450 Panel

| Compound | | | | | | | | |
|---|---|---|---|---|---|---|---|---|
| [10 μM] | 1A2* | 2B6* | 2C8* | 2C9* | 2C19* | 2D6* | 3A4* | 3A4\hat{ } |
| Reference-1 | 8.3 | 30.2 | 18.9 | 14.1 | 13.9 | 5.0 | 25.9 | 21.4 |
| Reference-2 | 0.8 | 13.6 | 41.5 | −1.6 | −0.9 | 3.4 | 16.6 | 15.7 |
| Reference-7 | 0.3 | 28.5 | 14.4 | 33.8 | 11.6 | −7.4 | 29.9 | 59.7 |
| Compound 9 | 4.8 | 14.8 | 4.0 | 6.8 | 8.2 | −3.7 | 16.3 | 26.8 |
| (Compound A) | | | | | | | | |

Controls

| | | | | | | | | |
|---|---|---|---|---|---|---|---|---|
| Fluvoxamine | 100.2 | — | — | — | — | — | — | — |
| Ticlopidine | — | 101.5 | — | — | — | — | — | — |
| Quercetin | — | — | 93.1 | — | — | — | — | — |
| Sulfaphenazole | — | — | — | 101.2 | — | — | — | — |
| Omeprazole | — | — | — | — | 61.6 | — | — | — |
| Quinidine | — | — | — | — | — | 99.7 | — | — |
| Ketoconazole | — | — | — | — | — | — | 110.1 | — |
| Ketoconazole | — | — | — | — | — | — | — | 105.5 |

*1A2 (phenacetin), 2B6 (bupropion), 2C8 (amodiaquine), 2C9 (Diclofenac), 2C19 (mephenytoin), 2D6 (dextromethorphan), 3A4 (midazolam)
\hat{ }3A4 (testosterone)

### Example 15

Cell-Based pERK Dose Response Study

Materials and Methods: A549 or A375 cells were seeded in 6-well plates at an appropriate seeding density on day 0. On day 2 or 3, after checking the health and confluency of cells, the media was aspirated and replaced with 1 mL media containing a predetermined concentration of compound and incubated for 2 hours. After 2 hours, the media was aspirated, and the cells washed 2 times with cold PBS. Cells were lysed on ice, with 50 μL 1× CST lysis buffer+1 mM PMSF for 5 minutes. After 5 minutes, cells were removed using a scraper and transferred to cold 1.5 mL tubes, and centrifuged for 10 min, 4° C., 14,000×g. Supernatant was gently removed and snap frozen in liquid nitrogen. Protein concentration of the lysate was determined using Bradford Reagent (analyzed using SpectraMax M2E) and diluted to 1 mg/mL or pERK or 1.5 mg/mL for pMEK analysis. Cell lysates were then analyzed for phosphor-ERK/total-ERK levels on a Jess system (ProteinSimple; Cat #JS3346) using the following antibodies: tERK1/2 (CST 4696; 1:50) and pERK1/2 (CST 4377; 1:50). For pMEK/tMEK, the following antibodies were used: tMEK1/2 (CST 4694; 1:15) and pMEK1/2 (CST 9154; 1:400). All dilutions were in Milk-free antibody diluent.

Mouse & Human Microsome (t_{1/2} min), Mouse & Human Clᵢₙₜ (μl/min/mg)

Test compounds were dissolved in DMSO to a concentration of 10 mM and further diluted to 100 μM using acetonitrile. Liver microsomes from selected species were incubated in duplicate with the test compound at a final concentration of 1 μM in 0.1 M potassium phosphate buffer (pH 7.4) containing 3.3 mM MgCl₂, 0.5 mg/ml microsomal protein, in the presence or absence of NADPH (1 mM). Incubations were performed at 37° C. in a total volume of 500 μl. Control incubations with reference substances were included for each experiment.

At different time points (t=0, 5, 15, 30, 45 min), 50 μl of the incubation mixture was transferred into a quench plate containing acetonitrile and internal standard (200 nM labetalol) cooled to 4° C. After the last time point, the quench plates were mixed thoroughly and centrifuged for 15 minutes at 3700 rpm and 10° C. (Eppendorf 5804R). The supernatant was transferred to new 96 well plates and subjected to LC-MS analysis. The disappearance of the parent compound was determined.

All sample analysis was performed using a Vanquish Horizon UHPLC-system equipped with an autosampler, a binary pump, a column compartment and a diode array detector coupled to a Q Exactive focus hybrid quadrupole-Orbitrap mass spectrometer (Thermo Fisher Scientific) equipped with heated electrospray ion source.

The percentage of test compound remaining was defined as the ratio of test compound peak area at a specific time point and the peak area in the t=0 min samples multiplied by 100%.

The metabolic stability was evaluated by plotting the natural logarithm of the percentage test compound remaining versus time and performing linear regression. Using this graph, the following parameters were calculated: elimination constant k (min⁻¹)=−slope, in vitro half-life (t_{1/2})=ln(2)/k, in vitro intrinsic clearance Clᵢₙₜ(in μl/min/mg protein) [ln(2)×incubation volume in μl/mg protein]/t_{1/2}.

Kinetic Solubility (PBS pH=7.4; 4 hr)

Test compounds were dissolved in DMSO to a concentration of 10 mM and further diluted to 100 μM in buffer (10 mM PBS, pH 7.4) in a 96 well plate at a final DMSO concentration of 1%. The plates were shaken for 4 h at room temperature in an Eppendorf Thermomixer. After incubation, the plates were centrifuged for 20 minutes at 4680 rpm. From the supernatant, 150 μL was transferred to a new 96 well plate and 50 μL DMSO was added to ensure continued dissolution. Samples were measured on LC-UV at injection volumes of 1 and 8 μL. Peak areas were determined and compared to peak areas obtained using calibration curves of the test compounds in DMSO. All sample analysis was performed using an Agilent 1290 HPLC-system equipped with an autosampler, a binary pump, a column compartment and a diode array detector.

e Log D (Lipophilicity; pH=7.4)

Test compounds were dissolved in DMSO at a concentration of 10 mM and further diluted with methanol:water 1:1. Samples were analyzed using gradient HPLC with three different isocratic mobile phases of 0.25% octanol in methanol (60, 65 and 70%) and 20 mM MOPS buffer (pH 7.4) with decylamine. If needed, (for low E log Doct compounds) the isocratic mobile phases were adjusted to e.g. 40, 45 and 50% methanol. Peaks were detected using a diode array at absorbance 220-320 nm.

Capacity factors data (k′_(tr−_t0)/t0) obtained at various amounts of methanol were extrapolated to 0% methanol and k′w values are determined using a linear procedure. E log Doct (7.4) is calculated using a series of reference standards with known Log D values. Each experiment was performed in triplicate.

All sample analysis was performed using an Agilent HPLC-system equipped with an autosampler, a binary pump, a column compartment and a diode array detector.

PAMPA (pH=7.4; Papp 10-6 cm/s)

PAMPA studies were conducted using the PAMPA Explorer kit (pION Inc.) and the double sink protocol (Avdeef, 2005). Stock solutions of all test compounds were dissolved in DMSO to a concentration of 10 mM. Each stock solution was diluted to 50 μM in pH 7.4 Prisma HT buffer (pION) and 200 μl was added to each well of the donor plate in triplicate. The polyvinylidene fluoride (PVDF, 0.45 μm) filter membrane on the acceptor plate was coated with 5 μl gastrointestinal tract lipid formulation (GIT-0, pION) and to each well of the acceptor plate, 200 μl of acceptor sink buffer (pION) was added. The acceptor filter plate was then carefully placed on top of the donor plate to form a sandwich. The sandwich was incubated at 25° C. for 4 h without stirring. UV-vis spectra of the solutions in the blank, reference, acceptor and donor plates were measured using a microplate reader (Tecan Infinite 200PRO M Nano Plus). Permeability values were calculated using the PAMPA explorer software v. 3.8.0.2 (pION). Control incubations with ketoprofen (low permeability) and verapamil (high permeability) were included in each experiment.

The results from a A549 (KRAS-G12S) pERK Dose Response study are also represented in Table 7.

TABLE 7

| | | | Compound 9 | |
|---|---|---|---|---|
| Attribute | Reference 1 | | (Compound A) | |
| pERK IC50 (A549) [12-point | 7.2 | nM | 19.8 | nM |

dose]

| | | |
|---|---|---|
| A549 pERK:tERK ratio [10 | 0.56 | 0.62 |

nM; 2 hr]

| | | |
|---|---|---|
| A375 pERK:tERK ratio | 0.59 | 0.71 |

[10 nM; 2 hr]

| | | |
|---|---|---|
| CRAF A549 pMEK:tMEK | 2.09 | 0.47 |

[100 nM; 2 hr]

| Mouse Microsome (t_{1/2} min) | 18 | min | 44 | min |
|---|---|---|---|---|
| Mouse Clint (μl/min/mg) | 78 | | 32 | |
| Human Microsome (t_{1/2} min) | >90 | min | 35 | min |
| Kinetic Solubility (PBS pH = | 36 | μM | >80 | μM |

7.4; 4 hr)

| | | |
|---|---|---|
| eLogD (lipophilicity; pH = | 3.6 | 3.0 |

7.4)

| | | |
|---|---|---|
| PAMPA (pH = 7.4; Pₐₚₚ 10⁻⁶ | 29 | 37 |

cm/s)

Reference 1 is Selumetinib

The results of a phospho-ERK Screen in A549 (KRAS G12S) study is described in Tables 8A and 8B below.

TABLE 8A
Phospho-ERK Screen in A549 (KRAS G12S): 10 μM for 2 hours

| Compound | pERK: total ERK (% relative to vehicle) |
|---|---|
| Vehicle | 100.00% |
| Reference 1 | 0.48% |
| Reference 2 | 1.26% |
| Reference 7 | 0.53% |
| Compound 9 | 0.60% |

Reference 1 is Selumetinib.
Reference 2 is 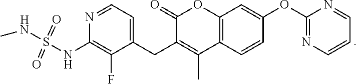 [SMILES: CNS(=O)(=O)Nc1nccc(Cc2c(C)c3ccc(Oc4ncccn4)cc3oc2=O)c1F]
Reference 7 is

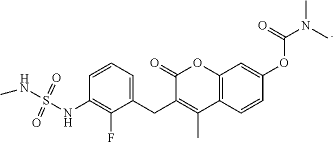

TABLE 8B
Phospho-ERK Screen in A549 (KRAS G12S): 10 μM for 2 hours

| Compound | pERK: total ERK (% relative to vehicle) |
|---|---|
| Vehicle | 100.00% |
| Reference 1 | 2.40% |
| Reference 2 | 2.59% |
| Reference 7 | 3.33% |
| Compound 9 | 1.81% |

Reference 1 is Selumetinib.
Reference 2 is 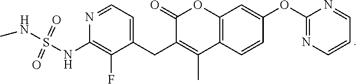 [SMILES: CNS(=O)(=O)Nc1nccc(Cc2c(C)c3ccc(Oc4ncccn4)cc3oc2=O)c1F]
Reference 7 is

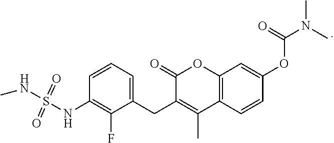

### Example 16

### Metabolite ID Study

Animal Studies: Compound A (Compound 9) was tested using six species hepatocytes in a metabolite ID study (T=60 min). The results of the study are represented in Table 9.

TABLE 9

| | Retention | | | | | | |
|---|---|---|---|---|---|---|---|
| m/z of | Time | % of total compound related material based on peak area | | | | | |
| [M + H]+ ion | Rt (min) | Mouse | Rat | Rabbit | Dog | Monkey | Human |
| Parent | 4.2 | 97 | 95 | 93 | 96 | 95 | 99 |
| Molecule | | | | | | | |
| Met-ID 1 | 3.8 | 0.8* | 0.8* | 0.2* | 0.8* | 1.4* | 0.9* |
| Met-ID 2 | 2.9 | 0.4* | 0.3* | 3.2* | 0.8* | 0.5 | 0.7* |
| Met-ID 3 | 3.7 | 0.4 | 1.0 | 0.1 | 0.0 | 0.9 | 0.3 |
| Met-ID 4 | 3.4 | 0.2 | 0.3 | 2.1 | 0.2 | 0.7 | 0.2 |
| Met-ID 5 | 3.6 | 0.1 | 0.2 | 0.0 | 0.0 | 0.0 | 0.0 |
| Met-ID 6 | 3.9 | 0.0 | 0.1 | 0.0 | 0.0 | 0.0 | 0.2 |
| Met-ID 7 | 2.7 | 1.3* | 1.3* | 1.0* | 1.5* | 0.5 | 0.9* |
| Met-ID 8 | 3.5 | 0.3* | 0.5* | 0.5* | 0.4* | 0.4 | 0.4* |

Bold and italic: main mass observed in that species
*also present in buffer incubations

Test compounds were dissolved in DMSO to a concentration of 10 mM, and further diluted to a 100 μM solution in DMSO:MQ 1:1. Cryopreserved hepatocytes from a selected species were thawed and incubated in duplicate with 10 μM compound 9 in Krebs-Henseleit buffer. Incubations were performed in a total volume of 350 μl, with 0.5×106 cells/ml. Control incubations with the reference substances verapamil, 7-hydroxycoumarin, propranolol and diltiazem were included for each species. Viability of hepatocytes was >70% after thawing.

At different time points, 50 μl of the incubation mixture was transferred into a quench plate containing acetonitrile and internal standard and cooled to 4° C. After the last time point (t=60 minutes), the quench plates were mixed thoroughly and centrifuged for 15 minutes at 3700 rpm and 10° C. (Eppendorf 5804R). The supernatant was transferred to new 96 well plates and subjected to LC-MS analysis. The disappearance of the parent compound was determined, and the formation of metabolites was evaluated. Metabolites present at >1% of total compound related material (based on peak area) were identified by interpretation of the LC-MS/MS data.

All sample analysis was performed using a Vanquish™ Horizon UHPLC-system equipped with an autosampler, a binary pump, a column compartment and a diode array detector coupled to a Q Exactive™ focus hybrid quadrupole-Orbitrap mass spectrometer (Thermo Fisher Scientific) equipped with heated electrospray ion source. MS settings were optimized for the flow rate. Full scan spectra were acquired in combination with data dependent MS2.

The formation and identification of metabolites was evaluated according to the following approach:

The spectra obtained from all samples were screened for the presence of m/z values that are absent in the blank incubations (incubations with hepatocytes without compound and with vehicle control, and incubations of compound in buffer without hepatocytes). T=60 min incubations were used as an initial screening. All possible metabolites observed were semi-quantified by MS response (peak area). MS2 spectra were interpreted to determine possible location of the metabolism reaction. The presence of metabolites identified was compared across the selected species.

Compound 9 was not extensively metabolized in hepatocytes from different species. As a percentage of total compound-related material in MS chromatograms of the hepatocyte incubations, approximately 92 to 98% of the parent compound was remaining (based on peak area). Primary metabolite shown for each species in bold font. Several masses found were also present in incubations with compound in buffer and were considered not to be metabolites.

### Example 17

### Colon-26 Model (KRAS G12D CRC): Efficacy & Safety

Female athymic BALB/c nude or BALB/c mice (Beijing Vital River Laboratory Animal Technology Co., Ltd.), approximately 8-10 weeks old were used in these studies. Animals were maintained in individually vented cages on a 12-h light-dark cycle. Food and water were available ad libitum. The Colon-26 tumor cells were maintained in vitro with RPMI1640 medium supplemented with 10% fetal bovine serum at 37° C. in an atmosphere of 5% CO₂ in air. The cells in exponential growth phase were harvested and quantitated by cell counter before tumor inoculation. Each mouse was inoculated subcutaneously in the right flank region with Colon-26 tumor cells (5×105) in 0.1 mL of PBS. Randomization was started when the mean tumor size reached approximately 125 mm³. A total of 84 tumor-bearing mice were enrolled in the study and allocated into 7 treatment groups (12 mice per group). Also 12 non-tumor-bearing mice were assigned as un-treated group.

Animals were treated with either vehicle (10% DMSO/90% [20% SBE-β-CD in saline], pH 5) or vehicle containing compounds at the indicated doses by gavage (p.o.) twice daily for approximately 28-42 days (BID×28-42 p.o.), or to ethical endpoints (BWL>20%; Median Tumor Volume (MTV)>2000 mm³; Individual TV>3000 mm³; clinical signs of unwell). Clinical signs of unwell was characterized, but not limited to the following. Severe dehydration, hypothermia, abnormal/labored respiration, lethargy, obvious pain, diarrhea, skin lesions, neurological symptoms, impaired mobility (not able to eat or drink) due to significant ascites and enlarged abdomen, astasia, continuous prone or lateral position, signs of muscular atrophy, paralytic gait, clonic convulsions, tonic convulsions, persistent bleeding from body orifice. Dosing as well as tumor and body weight measurements were conducted in a Laminar Flow Cabinet.

Animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm3 using the formula: V=(L×W×W)/2, where V is tumor volume, L is tumor length (the longest tumor dimension) and W is tumor width (the longest tumor dimension perpendicular to L). Results were presented as the median tumor volume, expressed in mm³+/−interquartile range, of each group. Tumor Growth Inhibition; TGI %=[1−(Ti/(Ci)]×100; where Ti as the median tumor volume of the treatment group on the measurement day, Ci as the median tumor volume of control group at the measurement day.

The body weights and tumor volumes were measured by using StudyDirector™ software (version 3.1.399.19).

The protocol and any amendment(s) or procedures involving the care and use of animals in this study were reviewed and approved by the Institutional Animal Care and Use Committee (IACUC) of CrownBio prior to execution. During the study, the care and use of animals were conducted in accordance with the regulations of the Association for Assessment and Accreditation of Laboratory Animal Care (AAALAC). Table 10 illustrate the results of this study.

TABLE 10
Colon-26 KRAS-G12D in Balb/c

| | Dose | | | |
|---|---|---|---|---|
| | (mg/kg | TGI | BWL | BWL |
| Drug | BID po) | (Day 10) | (Day 14) | (Day 17) |
| Vehicle | 0 | n/a | −26% | n/a |
| Reference 1 | 50 | 60.0% | 0% | −2% |
| Reference 2 | 50 | 61.6% | −2% | −2% |
| Compound 9 | 150 | 93.9% | 0% | 0% |

Reference 1 is Selumetinib.
Reference 2 is

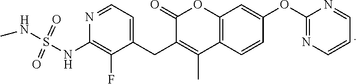 [SMILES: CNS(=O)(=O)Nc1nccc(Cc2c(C)c3ccc(Oc4ncccn4)cc3oc2=O)c1F]

### Example 18

### Pharmacokinetics

Female mice (either athymic BALC/c nude, or BALB/c mice) were inoculated with a single subcutaneous injection of 0.2 ml of mouse Colon-26 cell suspension (1×105 cells per animal) in the inguinal region. Colon26 (C26) tumor bearing mice received a single dose (3 mice/treatment) of vehicle (10% DMSO/90% [20% SBE-R-CD in saline], pH 5) containing compounds at 100 mg/kg by gavage (p.o.) and 2-hour post dose humanely euthanized.

Blood was obtained through cardiac puncture into K+EDTA tubes, mixed by inversion and centrifuged to obtain the plasma. Tissues were excised, cleaned of surrounding tissues. All samples immediately snap frozen in liquid N2 prior to LC-MS/MS analysis (using Waters HSS T3 2.1×50 mm (1.8 um) LC column, and an API-6500 Electrospray MS unit). The results of this study are represented in Table 11.

TABLE 11
Pharmacokinetics Summary

| Compound | Plasma | Liver | Tumor | Muscle |
|---|---|---|---|---|
| Reference 2 | 5670 ng/ml | 1990 ng/g | 819 ng/g | 550 ng/g |
| Compound 9 | 407 ng/ml | 1260 ng/g | 1350 ng/g | 363 ng/g |

Reference 2 is

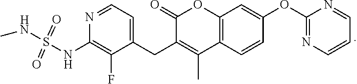 [SMILES: CNS(=O)(=O)Nc1nccc(Cc2c(C)c3ccc(Oc4ncccn4)cc3oc2=O)c1F]

### Example 19

### Pharmacokinetic Profile for Compound A

Female mice (either athymic BALC/c nude, or BALB/c mice) were inoculated with a single subcutaneous injection of 0.1 ml of mouse Colon-26 cell suspension (1×105 cells per animal) in the right flank region. Colon26 (C26) tumor bearing mice received a single dose (3-4 mice/treatment) of vehicle (10% DMSO/90% [20% SBE-R-CD in saline], pH 5) containing compounds at indicated dose by gavage (p.o.) or intravenous administration (i.v.) and at various time points (0.16, 0.5, 1, 2, 4, 8, 12, 16, 24-hour) post dose humanely euthanized. Blood was obtained through cardiac puncture into K+EDTA tubes, mixed by inversion and centrifuged to obtain the plasma. Tissues were excised, cleaned of surrounding tissues. All samples immediately snap frozen in liquid N2 prior to LC-MS/MS analysis (using Agilent Poroshell-120 EC-C18 (4.0 μm) 2.1×50 mm, and a Water+API-4000™ Electrospray MS unit). Table 12A and 12B describe a single dose PK profile (tumor).

TABLE 12A
Single Dose PK Profile (Plasma), compound A

| PK Parameter | 25 mpk i.v. | 25 mpk p.o. | 100 mpk p.o. |
|---|---|---|---|
| T_{1/2} (hr) | 0.63/2.39 | 1.32/1.23 | 1.22/1.38 |
| Tₘₐₓ (hr) | 0.50/0.16 | 0.50/0.50 | 0.50/0.16 |
| Cₘₐₓ (ng/mL) | 6,520/10,930 | 4,640/2,317 | 30,067/14,200 |
| AUC₀₋ₜ (ng · hr/mL) | 5,807/7,730 | 4,392/3,645 | 44,548/31,063 |
| AUC_{0-∞} (ng · hr/mL) | 5,808/7,735 | 4,404/3,650 | 44,566/31,071 |
| Bioavailability (%) | n/a/n/a | 75.6/47.0 | 191.8/100.0 |

TABLE 12B
Single Dose PK Profile (Tumor), Compound A

| PK Parameter | 25 mpk i.v. | 25 mpk p.o. | 100 mpk p.o. |
|---|---|---|---|
| T_{1/2} (hr) | 3.46/— | 1.28/— | 1.97/— |
| Tₘₐₓ (hr) | 0.50/0.50 | 0.50/1.00 | 0.50/1.00 |
| Cₘₐₓ (ng/mL) | 7,790/6,750 | 4,577/1,917 | 30,333/12,560 |
| AUC₀₋ₜ (ng · hr/mL) | 8,278/8,315 | 4,892/3,461 | 51,661/36,856 |
| AUC_{0-∞} (ng · hr/mL) | 8,288/8,363 | 4,903/— | 51,711/37,087 |
| Bioavailability (%) | n/a/n/a | 59.1/42.0 | 156.0/111.0 |

### Example 20

### Drug Profiles

Table 13 describes the attributes of two compounds and a reference compound.

TABLE 13

| Attribute | Reference 2 | | Compound 9 | |
|---|---|---|---|---|
| pERK IC50 (A549) [12-point dose] | 7.2 | nM | 19.8 | nM |
| A549 pERK:tERK ratio [10 nm; 2 hr] | 0.56 | | 0.62 | |
| A375 pERK:tERK ratio [10 nM; 2 hr] | 0.59 | | 0.71 | |
| CRAF A549 pMEK:tMEK [100 nM; 2 hr] | 2.09 | | 0.47 | |
| Mouse Microsome (t_{1/2} min) | 18 | min | 44 | min |
| Mouse Clint (μl/min/mg) | 78 | | 32 | |
| Human Microsome (t_{1/2} min) | >90 | min | 35 | min |
| Human Clint (μl/min/mg) | <15 | | 40 | |
| Kinetic Solubility (PBS pH = 7.4; 4 hr) | 36 | μM | >80 | μM |

| | | |
|---|---|---|
| eLogD (lipophilicity; pH = 7.4) | 3.6 | 3.0 |
| PAMPA (pH = 7.4; Papp 10 − 6 cm/s) | 29 | 37 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1-55. (canceled)

56. A method of treating a RAS-mutated solid tumor, comprising repeatedly administering a therapeutically effective amount of a dual-MEK inhibitor compound to a patient in need thereof on a dosing schedule with sequential doses of the dual-MEK inhibitor being administered at intervals that are about 7 to about 18 times the duration of the human plasma drug half-life of the dual-MEK inhibitor compound in the patient.

57. The method of claim 56, wherein the dual-MEK inhibitor is administered at intervals that are about 7 to about 10 times the duration of the human plasma drug half-life of the dual-MEK inhibitor compound.

58. The method of claim 56, wherein the dual-MEK inhibitor compound reaches a drug trough level in the patient within about 24-hours after oral administration of the dual-MEK inhibitor compound.

59. The method of claim 56, wherein the dual-MEK inhibitor compound is administered once per day (QD).

60. The method of claim 56, wherein the dual-MEK inhibitor compound has a human plasma half life of about 1 to about 3 hours.

61. The method of claim 56, wherein the RAS-mutated solid tumor is a KRAS-mutated solid tumor.

62. The method of claim 61, wherein the KRAS-mutated solid tumor is a KRAS G12C, G12D, G12S, or G12V mutated solid tumor.

63. The method of claim 57, wherein the dual-MEK inhibitor compound has a human plasma half life of 1-3 hours and the dual-MEK inhibitor compound is administered once per day (QD).

64. The method of claim 63, wherein the RAS-mutated solid tumor is a KRAS-mutated solid tumor.

65. The method of claim 64, wherein the KRAS-mutated solid tumor is a KRAS G12C, G12D, G12S, or G12V mutated solid tumor.

66. A method of treating a RAS-mutated solid tumor, comprising the step of administering a therapeutically effective amount of a compound of Formula (IIb), 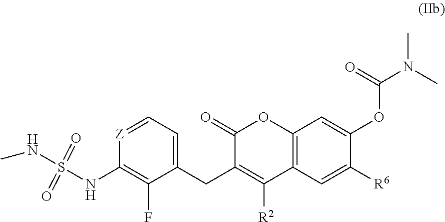
wherein,
Z is C or N;
R₂ is L;
L is —Z₁-Z₂;
Z₁ is —CH₂—;
Z₂ is —NR⁵R^{5′},
R⁵ and R^{5′} are each independently methyl, ethyl, propyl, iso-propyl, n-butyl, iso-butyl, sec-butyl, or t-butyl; and
R⁶ is H, halogen, or C₁ to C₆ alkyl,
to a patient in need thereof on a dosing schedule with sequential doses of the compound being repeatedly administered at intervals that are about 7 to about 18 times the duration of the human plasma drug half-life of the compound in the patient.

67. The method of claim 66, wherein the compound is administered at intervals that are about 7 to about 10 times the duration of the human plasma drug half-life of the compound.

68. The method of claim 67, wherein the compound has a human plasma half life of about 1 to about 3 hours.

69. The method of claim 68, wherein the RAS-mutated solid tumor is a KRAS-mutated solid tumor.

70. The method of claim 69, wherein the KRAS-mutated solid tumor is a KRAS G12C, G12D, G12S, or G12V mutated solid tumor.

71. The method of claim 70, wherein the dual-MEK inhibitor compound is orally administered once per day (QD).

72. The method of claim 66, wherein the dual-MEK inhibitor compound is orally administered once per day (QD).

73. The method of claim 66, wherein the therapeutically effective amount is a once daily dose of about 160 mg to about 320 mg of the compound per day.

74. A method of treating a KRAS-mutated solid tumor comprising administering about 160 mg to about 320 mg per day of Compound 9, 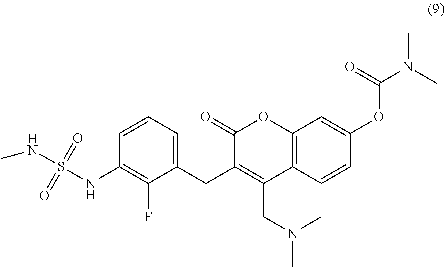
or a pharmaceutically acceptable salt thereof, to a patient in need thereof, on a dosing schedule with sequential doses of Compound 9 administered at intervals that are about 7 to about 10 times the duration of the human plasma drug half-life of compound (9).

75. The method of claim 74, wherein the KRAS-mutated solid tumor is a KRAS G12C, G12D, G12S, or G12V mutated solid tumor.
